SEQUENCE LISTING (44 sequences)

SEQ ID NO: 1 - DNA, 19 bp, synthetic construct
  source 1..19: mol_type = other DNA
gagggctggactgcgaact

SEQ ID NO: 2 - DNA, 19 bp, synthetic construct
  source 1..19: mol_type = other DNA
cagtgcccgaaacccacac

SEQ ID NO: 3 - DNA, 20 bp, synthetic construct
  source 1..20: mol_type = other DNA
tttggaagctgctggggaag

SEQ ID NO: 4 - DNA, 21 bp, synthetic construct
  source 1..21: mol_type = other DNA
ctccgtgaagggataaccagg

SEQ ID NO: 5 - DNA, 20 bp, synthetic construct
  source 1..20: mol_type = other DNA
accaacctgtccaagttccg

SEQ ID NO: 6 - DNA, 22 bp, synthetic construct
  source 1..22: mol_type = other DNA
ggagttcaaagaagccttcagc

SEQ ID NO: 7 - DNA, 24 bp, synthetic construct
  source 1..24: mol_type = other DNA
acatcatcacccacggagaagaga

SEQ ID NO: 8 - DNA, 20 bp, synthetic construct
  source 1..20: mol_type = other DNA
ggtggatgaggagcgatacg

SEQ ID NO: 9 - DNA, 22 bp, synthetic construct
  source 1..22: mol_type = other DNA
ggaggacaccgagaaggaaaac

SEQ ID NO: 10 - DNA, 21 bp, synthetic construct
  source 1..21: mol_type = other DNA
gctttctgggtggactcaagt

SEQ ID NO: 11 - DNA, 20 bp, synthetic construct
  source 1..20: mol_type = other DNA
gctccggcttcaagacactc

SEQ ID NO: 12 - DNA, 15 bp, synthetic construct
  source 1..15: mol_type = other DNA
cccggaggccgaggt

SEQ ID NO: 13 - DNA, 21 bp, synthetic construct
  source 1..21: mol_type = other DNA
tcacctgtgagaattgactgg

SEQ ID NO: 14 - DNA, 21 bp, synthetic construct
  source 1..21: mol_type = other DNA
ttggaagtggactccaagaaa

SEQ ID NO: 15 - DNA, 19 bp, synthetic construct
  source 1..19: mol_type = other DNA
ggtgactggagcgccttag

SEQ ID NO: 16 - DNA, 21 bp, synthetic construct
  source 1..21: mol_type = other DNA
agccaaccttaactgaggagt

SEQ ID NO: 17 - DNA, 21 bp, synthetic construct
  source 1..21: mol_type = other DNA
tgattccaacgccaccaattc

SEQ ID NO: 18 - DNA, 18 bp, synthetic construct
  source 1..18: mol_type = other DNA
gactgacactcgcagccc

SEQ ID NO: 19 - DNA, 20 bp, synthetic construct
  source 1..20: mol_type = other DNA
tcagacgtgcagtacacgga

SEQ ID NO: 20 - DNA, 20 bp, synthetic construct
  source 1..20: mol_type = other DNA
tatccccgggactaagacgg

SEQ ID NO: 21 - DNA, 20 bp, synthetic construct
  source 1..20: mol_type = other DNA
agggatggtcattgccttgg

SEQ ID NO: 22 - DNA, 20 bp, synthetic construct
  source 1..20: mol_type = other DNA
gtggacctgacctgccgtct

SEQ ID NO: 23 - DNA, 19 bp, synthetic construct
  source 1..19: mol_type = other DNA
tttgcacccctcccaattc

SEQ ID NO: 24 - DNA, 20 bp, synthetic construct
  source 1..20: mol_type = other DNA
ggagacccagcagcctcaaa

SEQ ID NO: 25 - DNA, 20 bp, synthetic construct
  source 1..20: mol_type = other DNA
gatgggaggaggggagagga

SEQ ID NO: 26 - DNA, 20 bp, synthetic construct
  source 1..20: mol_type = other DNA
ttcacagtcaccgtcttccc

SEQ ID NO: 27 - DNA, 20 bp, synthetic construct
  source 1..20: mol_type = other DNA
tcattcaagcccttcgtgcc

SEQ ID NO: 28 - DNA, 20 bp, synthetic construct
  source 1..20: mol_type = other DNA
aaagagcgtgaggaagacgg

SEQ ID NO: 29 - DNA, 24 bp, synthetic construct
  source 1..24: mol_type = other DNA
attggaacatggcctctggatgga

SEQ ID NO: 30 - DNA, 23 bp, synthetic construct
  source 1..23: mol_type = other DNA
gcttcaggtccttaatctccctg

SEQ ID NO: 31 - DNA, 21 bp, synthetic construct
  source 1..21: mol_type = other DNA
tcaaactttttcttgcggccc

SEQ ID NO: 32 - DNA, 21 bp, synthetic construct
  source 1..21: mol_type = other DNA
gagggcaatccgtcttcatcc

SEQ ID NO: 33 - DNA, 20 bp, synthetic construct
  source 1..20: mol_type = other DNA
tgcgcttggaggaaatagcc

SEQ ID NO: 34 - DNA, 25 bp, synthetic construct
  source 1..25: mol_type = other DNA
tcaggtcccagtaaaatctgaagtc

SEQ ID NO: 35 - DNA, 20 bp, synthetic construct
  source 1..20: mol_type = other DNA
agaaagagtgtgcagcggat

SEQ ID NO: 36 - DNA, 20 bp, synthetic construct
  source 1..20: mol_type = other DNA
cgaagacgagatccagttcc

SEQ ID NO: 37 - DNA, 20 bp, synthetic construct
  source 1..20: mol_type = other DNA
gcgcacatgagagattggga

SEQ ID NO: 38 - DNA, 21 bp, synthetic construct
  source 1..21: mol_type = other DNA
ggcaagttgattggagggatg

SEQ ID NO: 39 - DNA, 22 bp, synthetic construct
  source 1..22: mol_type = other DNA
gaggagtccataggcgattact

SEQ ID NO: 40 - DNA, 24 bp, synthetic construct
  source 1..24: mol_type = other DNA
cagtccagaatcattgcacctgag

SEQ ID NO: 41 - DNA, 21 bp, synthetic construct
  source 1..21: mol_type = other DNA
gggagccttccgttctagagt

SEQ ID NO: 42 - DNA, 20 bp, synthetic construct
  source 1..20: mol_type = other DNA
caccatcaccccctgatgtc

SEQ ID NO: 43 - DNA, 20 bp, synthetic construct
  source 1..20: mol_type = other DNA
atcctccataggaccagccc

SEQ ID NO: 44 - DNA, 20 bp, synthetic construct
  source 1..20: mol_type = other DNA
ggaggagtgggtgtcgctgt

(19) United States
(12) Patent Application Publication
(10) Pub. No.: US 2024/0026297 A1
(43) Pub. Date: Jan. 25, 2024

(54) THREE-DIMENSIONAL CULTURE SYSTEM FOR GENERATING CARDIAC SPHEROIDS

(71) Applicant: THE UAB RESEARCH FOUNDATION, Birmingham, AL (US)

(72) Inventors: Asher Max Kahn-Krell, Birmingham, AL (US); Jianyi Zhang, Vestavia, AL (US)

(21) Appl. No.: 18/351,561

(22) Filed: Jul. 13, 2023

Related U.S. Application Data
(60) Provisional application No. 63/368,296, filed on Jul. 13, 2022.

(51) Int. Cl. C12N 5/077; A61P 9/10

(52) U.S. Cl. CPC C12N 5/0657; A61P 9/10; C12N 2513/00; C12N 2506/45; C12N 2510/00

(57) ABSTRACT

Disclosed herein is a simple and reproducible 3D-culture-based process for generating cardiac spheroids containing all four cardiac-cell types (cardiomyocytes, endothelial cells, smooth muscle cells, and cardiac fibroblasts) that is compatible with a wide range of applications and research equipment. Subsequent experiments demonstrated that the inclusion of vascular cells and cardiac fibroblasts was associated with an increase in spheroid size, a decline in apoptosis, an improvement in sarcomere maturation and a change in CM bioenergetics. These suggest a three-dimensional (3D) environment with endothelial cells, smooth muscle cells, and cardiac fibroblasts which promoted CM maturation and electrical activity.

### CROSS-REFERENCE TO RELATED APPLICATIONS

This application claims benefit of U.S. Provisional Application No. 63/368,296, filed Jul. 13, 2022, which is hereby incorporated herein by reference in its entirety.

### SEQUENCE LISTING

This application contains a sequence listing filed in ST.26 format entitled “222119-1190 Sequence Listing” created on Jul. 4, 2023, having 39,747 bytes. The content of the sequence listing is incorporated herein in its entirety.

### BACKGROUND

The development of efficient protocols for differentiating human induced-pluripotent stem cells (hiPSCs) into cardiomyocytes (CMs), endothelial cells (ECs), smooth muscle cells (SMCs), and cardiac fibroblasts (CFs) has led to the establishment of an array of utilizations in the field of cardiac regeneration. Although early proposed applications focused on direct injections and the transplantation of engineered cardiac tissues and sheets to resupply cardiomyocytes, lost to injuries such as myocardial infarction (MI), the uses of these cardiac surrogates have expanded to include in-vitro modeling of myocardial disease, drug screening, and exosome production. A wide range of formats has been explored to fulfill the needs of each potential application including patches, sheets, spheres, wires, and decellularized tissues. The utilization of these products and the needs of each use play a key role in the ideal option for each situation. However, there is a crucial need to establish tools for multiple applications related to MI and heart failure that can be easily reproduced and scaled.

### SUMMARY

Cardiomyocytes (CMs), endothelial cells (ECs), smooth muscle cells (SMCs), and cardiac fibroblasts (CFs) differentiated from human induced-pluripotent stem cells (hiPSCs) are the fundamental components of cell-based regenerative myocardial therapy and can be used as in-vitro models for mechanistic studies and drug testing. However, newly differentiated hiPSC-CMs tend to more closely resemble fetal CMs than the mature CMs of adult hearts, and current techniques for improving CM maturation can be both complex and labor-intensive. Disclosed herein is a simple and reproducible 3D-culture-based process for generating cardiac spheroids containing all four cardiac-cell types (cardiomyocytes, endothelial cells, smooth muscle cells, and cardiac fibroblasts) that is compatible with a wide range of applications and research equipment. Subsequent experiments demonstrated that the inclusion of vascular cells and cardiac fibroblasts was associated with an increase in spheroid size, a decline in apoptosis, an improvement in sarcomere maturation and a change in CM bioenergetics. These suggest a three-dimensional (3D) environment with endothelial cells, smooth muscle cells, and cardiac fibroblasts which promoted CM maturation and electrical activity.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1. Experimental design. (A) hiPSCs were differentiated into CMs, ECs, SMCs, and CFs via established methods. (B) Spheroids containing all four cell types (C4 spheroids) were produced by combining a 4:2:1:1 ratio of CMs, ECs, SMCs, and CFs in 96-well U plates and allowing the cells to fuse for 7 days; then, (C) the spheroids were transferred to 6-well plates and (D) cultured with shaking for to 60 days. (E) Procedures for spheroid manufacture and experimental analyses are displayed on a timeline.

FIGS. 2A to 2F. Size and histological assessments. FIG. 2A. C4 spheroids were generated from the indicated CM population sizes (0.6×10⁵ black; 0.8×10⁵ purple; 1.0×10⁵ pink; 1.2×10⁵ orange; 1.4×10⁵ yellow), and spheroid diameters were measured on D7 and D30; results were displayed as box-whisker plots. FIG. 2B. C4 spheroids were cut into 10-μm sections, and the sections were stained via terminal deoxynucleotidyl transferase dUTP nick end labeling (TUNEL); then, apoptosis was quantified as the percentage of TUNEL-positive cells in 4 spheroids/group. FIG. 2C. Images of C1, C3, and C4 spheroids generated from an initial population of 1×10⁵ CMs were obtained each day from DO-D7 (bar=500 μM). FIG. 2D. Spheroid diameters of all 3 groups (C1 black; C3 yellow; C4 blue) were measured on D7, D30, and D60; results are displayed as violin plots. FIG. 2E. Spheroids were cut into 10-μm sections and stained with Masson-trichrome; representative images are displayed for every tenth sequential section from a C4 spheroid on D60 (bar=500 μM). FIG. 2F. Representative images are displayed for the centermost sections from C1, C3, and C4 spheroids at the indicated time points (bar=500 μM for the first three columns, and bar=100 μM for the far-right column); muscle fibers appear red and collagen fibers appear blue. (*p<0.05, **p<0.01, ***p<0.001; n>30 per group; p-values determined as mean+/−SEM).

FIGS. 3A to 3E. Apoptosis and cell-occupancy measurements. FIG. 3A. C1, C3, and C4 spheroids were collected at the indicated time points and cut into 10-μm sections; then, the surface areas of every tenth section were measured, and the centermost section (i.e., the one with the largest surface area) was TUNEL-stained. Nuclei were counterstained with DAPI, and apoptosis was quantified as the percentage of TUNEL-positive cells. Representative images are displayed for the centermost sections from each group on D60. FIG. 3B. Centermost sections were stained with cTnT, CD31, and TE-7 antibodies to visualize CMs, ECs, and CFs, respectively, and nuclei were counterstained with DAPI; representative images are displayed for each group on D7, D30, and D60. FIGS. 3C-3D. CM, EC, and CF occupancy were quantified as the percentage of total surface area that was positive for expression of the corresponding m 509 arker protein and summarized (FIG. 3C) for C1, C3, and C4 on D7 and (FIG. 3D) for C4 on D7, D30, and D60. FIG. 3E. Representative sections from a C4 spheroid on D60 are displayed at high magnification to demonstrate the presence of elongated ECs. (*p<0.05, **p<0.01, ***p<0.001; n>6 per group).

FIGS. 4A to 4E. Changes in patterns of gene expression during culture. FIGS. 4A-4B. The abundance of beta MHC, alpha MHC, MLC 2v, MLC 2a, TNNI3, and TNNI1 (FIG. 4A) mRNA and (FIG. 4B) protein were evaluated via qPCR and Western blot, respectively, in C4 spheroids at the indicated time points (D1 purple; D7 blue; D30 teal; D60 green); then, the ratios of expression for the MHC (top), MLC (middle), and TNNI isoforms (bottom) were calculated. A representative Western blot of protein expression is displayed on the right of panel B. FIGS. 4C-4D. The abundance of (FIG. 4C) TNNI3, N-cadherin, and PPARGC1a mRNA and of (FIG. 4D) CD31, alpha-SMA, and FAP mRNA was measured via qPCR in C4 spheroids at the indicated time points. FIG. 4E. The ratios of mRNA abundance for the MHC, MLC, and TNNI isoforms was calculated for C1, C3, and C4 spheroids on D60 (C1 black; C3 yellow; C4 blue). mRNA measurements were normalized to intrinsic GAPDH mRNA abundance, and protein measurements were normalized to intrinsic beta actin abundance. (*p<0.05, **p<0.01; n>4 per group).

FIGS. 5A to 5D. Patterns of metabolic, electrical, and cell-cycle gene expression in C1, C3, and C4 spheroids. The magnitude of expression for genes that contribute to CM (FIG. 5A) electrical conduction (HCN4, N cadherin, SERCA, Ryr2, Cx43, CACNA1C), (FIG. 5B) metabolism (PPARGC1a, CKMT2), and (FIG. 5C) cell cycle activity (CDK6) was evaluated in C1, C3, and C4 spheroids on D60 (C1 black; C3 yellow; C4 blue) via qPCR; measurements were normalized to intrinsic GAPDH mRNA abundance. FIG. 5D. ATP abundance, the NAD/NADH ratio, and cellular cAMP was evaluated in C1, C3, and C4 spheroids on D60 (C1 black; C3 yellow; C4 blue) via luminescent ATP detection assay, colorimetric assay, and direct cAMP ELISA respectively (*p<0.05, **p<0.01; n>4 per group).

FIGS. 6A to 6C. TEM assessments of the ultrastructure of C1, C3, and C4 spheroids. FIG. 6A. Whole C1, C3, and C4 spheroids on day 60 were sectioned and imaged via TEM (bar=2 μM for the top row, and bar=1 μM for the bottom row); ZL: Z-line, ML: M-line, MT: mitochondria, GJ: gap junction, N: nucleus. (B) Sarcomere lengths were calculated by measuring the distance between adjacent Z-lines. FIG. 6C. Sarcomere widths were measured as the length of each uninterrupted Z-line. (*p<0.05, **p<0.01, ***p<0.001; n>5 spheroids per group).

FIGS. 7A to 7D. MEA assessments. FIG. 7A. Whole C1, C3, and C4 spheroids (C1 black; C3 yellow; C4 blue) were attached to a 4×4 MEA; then, field potentials and impedance were measured and used to calculate (FIG. 7B) beat period, spike amplitude, field potential duration (FPD), conduction velocity, beat amplitude, and excitation. FIG. 7C. C1, C3, and C4 spheroids (C1 black; C3 yellow; C4 blue) were dissociated on D30; then, CMs were collected and plated on the MEA electrodes. Field potentials and impedance were measured with and without pacing at 3 hz (330 ms or 180 bpm) and used to calculate (FIG. 7D) beat period, spike amplitude, FPD, conduction velocity, and the action potential duration until 50% and 90% recovery (APD50 and APD90), respectively. (E) MEA field potential traces for C1, C3, and C4 whole organoids only showing the 4 electrodes that were used for analysis. (F) MEA field potential traces of dissociated C1, C3, and C4 organoids after undergoing LEAP induction (*p<0.05, **p<0.01, ***p<0.001; n>6 per group).

FIG. 8. Characterization data from iPSC-ECs illustrating their purity and expression of key markers.

FIG. 9. Characterization data from iPSC-SMCs illustrating their purity and expression of key markers.

FIG. 10. Characterization data from iPSC-CFs illustrating their purity and expression of key markers.

FIG. 11. Characterization data from iPSC-CM spheroids after dissociation illustrating their purity and expression of key markers.

FIG. 12. The magnitude of expression for genes that contribute to CM (A) electrical conduction (HCN4, N-cadherin, SERCA, Ryr2, Cx43, CACNA1C), (B) metabolism (PPARGC1a, CKMT2), and (C) cell-cycle activity (CDK6) was evaluated in C1, C3, and C4 spheroids on D7 (C1 black; C3 yellow; C4 blue) via qPCR; measurements were normalized to intrinsic GAPDH mRNA abundance (*p<0.05; n>4 per group).

FIG. 13. Whole C1, C3, and C4 spheroids on day 60 were sectioned and imaged via TEM (bar=1 μM).

FIGS. 14A to 141. Characterization of ^{HLA-KO/CCND2-OE}hiPSC-CMs. FIG. 14A.Cyclin D2 (CCND2) gene expressions in ^{HLA-KO}hiPSC-CMs and ^{HLA-KO/CCND2-OE}hiPSC-CMs were determined by qRT-PCR. FIG. 14B. Western Blot for CCND2 and β-tubulin proteins in ^{HLA-KO}hiPSC-CMs and ^{HLA-KO/CCND2-OE}hiPSC-CMs. FIG. 14C. Quantification of CCND2 protein expression. FIG. 14D. Western Blot images of CIITA, β2M, and β-tubulin in WT-CMs, ^{HLA-KO}hiPSC-CM s and ^{HLA-KO/CCND2-OE}hiPSC-CM s. FIG. 14E. Quantification of CIITA and β2M protein expressions. FIG. 14F. Co-fluorescence immunostaining for cTnT and pH3 protein expressions in ^{HLA-KO}hiPSC-CM s and ^{HLA-KO/CCND2-OE}hiPSC-CMs. FIG. 14G. Quantification of pH3⁺ CMs. FIG. 14H. Co-fluorescence immunostaining for cTnT and aurora-B protein expressions in ^{HLA-KO}hiPSC-CMs and ^{HLA-KO/CCND2-OE}hiPSC-CMs. FIG. 14I. Quantification of Aurora-Bk CM under cytokinesis. Scale bar=20 μm. All data were presented as mean±SEM. (*p<0.05, ** p<0.01).

FIGS. 15A to 15E. Characterization of ^{HLA-KO/CCND2-OE}hiPSC-CM Spheroids. FIG. 15A. Representative image of CM Spheroids grown in a shaker flask. FIG. 15B. Bright-field images of CM Spheroids at a low magnification. FIG. 15C. Yield of CMs generated in each suspension differentiation. FIG. 15D. Purity of CM as evaluated by flow cytometry. FIG. 15E. Confocal images of spheroid stained with cTnT and α-Sarcomeric Actinin (α-SA). Scale bar=20 μm. All the data were shown as mean±SEM.

FIGS. 16A to 16G. ^{HLA-KO/CCND2-OE}hiPSC-CM spheroids transplantation improved the cardiac function in porcine hearts after I/R. FIG. 16A. Representative cardiac magnetic resonance images of the porcine hearts which were used for the assessments of cardiac function and scar size. FIG. 16B. Quantification of the left ventricular end-diastolic volume (LVESV). FIG. 16C. Quantification of the left ventricular End-systolic volume (LVEDV). FIG. 16D. Quantification of the left ventricular ejection fraction (LVEF). FIG. 16E. Quantification of the scar sizes. Gross anatomic images of cardiac slices in I/R+ Vehicle (FIG. 16F) and I/R+spheroid (FIG. 16G) treated pig hearts at 28 days after I/R and treatment. Values are presented as means±SEM (** p<0.01).

FIGS. 17A to 17M. ^{HLA-KO/CCND2-OE}hiPSC-CM spheroids implantation induced endogenous pig CM proliferation after IR. FIG. 17A. Representative engraftment of hiPSC-CMs which were stained positively for human nuclei antigen (HNA) and cTnT in the pig hearts at 1 week after transplantation. FIG. 17B. Representative engraftment of hiPSC-CMs which were stained positively for human Ku80 and α-sarcomeric actinin (α-SA) in the pig hearts at 1 week after transplantation. Scale bar=100 μm. FIG. 17C. Representative mapping image showing a heart tissue slice with implanted spheroids which flashed bright when excited by blue light during optical mapping. FIG. 17D. The corresponding calcium transient signal (CaT) from one spheroid in optical mapping. FIG. 17E. Representative images of fluorescence immunostaining for pH3 and cTnI protein expressions at border zone of injury in the pig hearts at 1 week after I/R. FIG. 17F. Quantification of pig pH3⁺ CM at border zone of injury in the pig hearts at 1 week after I/R. FIG. 17G. Representative images of fluorescence immunostaining for aurora-B kinase and cTnI protein expressions at border zone of injury in the pig hearts at 1 week after I/R. FIG. 17H. Quantification of pig aurora B⁺ CM under cytokinesis at border zone of injury in the pig hearts at 1 week after IR. Scale Bar=50 μm. FIG. 17I. Western Blot analysis of pig proteins isolated from the border zone of injury for expressions of phosphorylated LATS (pLATS), phosphorylated YAP (p-YAP), LATS, YAP, and GAPDH. Quantification of p-LATS/LATS (FIG. 17J) and p-YAP/YAP (FIG. 17K) ratios. FIG. 17L. Representative images of fluorescence immunostaining for YAP and cTnT protein expressions at border zone of injury in the pig hearts at 1 week after I/R. FIG. 17M. Quantification of pig YAP⁺ CM nuclei at border zone of injury in the pig hearts at 1 week after I/R. Scale Bar=50 μm. Data are presented as the mean±SEM. p<0.05).

FIGS. 18A to 18L. The cell-cycle-specific Autoencoder identified cycling CM cluster1 (C.CM), which had colocalized cell-cycle markers. FIG. 18A. CM UMAP plot, where the cells were grouped based on the animal group. FIG. 18B. CM UMAP plot to show C.CM. Localization of cell-cycle marker on UMAP plot: AURKB (FIG. 18C), MKI67 (FIG. 18D), INCENP (FIG. 18E), CDCA8 (FIG. 18F), and BIRC5 (FIG. 18G). FIG. 18H. Percentage of C.CM in porcine CMs in all animal groups. The differential enrichment analysis, computed by the sparse model score, suggested that HIPPO/YAP (FIG. 18I), TGFβ (FIG. 18J), and MAPK (FIG. 18K) signaling pathways of porcine CM were significantly upregulated. FIG. 18L. The differential enrichment analysis of YAP1 gene expression in porcine CMs. (* p<0.05)

FIGS. 19A to 19G. Follistatin promoted CM proliferation through up-regulation of YAP protein expression. FIG. 19A. Representative images of fluorescence immunostaining of hiPSC-CMs for protein expressions of aurora-B kinase and cTnT after treated with 0, 100, 200, and 250 ng/mL follistatin for 2 days. FIG. 19B. Quantification of aurora-B kinase expressing hiPSC-CMs under cytokinesis. Scale bar=50 μm. FIG. 19C. Quantification of hiPSC-CM number treated with or without 200 ng/mL follistatin for 16 days. FIG. 19D. Western Blot analysis for p-YAP and YAP protein expression hiPSC-CMs induced by 200 ng/mL follistatin. Quantification of the ratios of pYAP/GAPDH (FIG. 19E), YAP/GAPDH (FIG. 19F), and pYAP/YAP (FIG. 19G). Data are presented as the mean±SEM. (* p<0.05).

FIG. 20. A schematic presentation of in vivo study using porcine heart model of ischemia/reperfusion.

FIGS. 21A to 21D. Characterization of ^{HLA-KO/CCND2-OE}hiPSCs. FIG. 21A. Phase contrast image of a ^{HLA-KO/CCND2-OE}hiPSC colony. FIG. 21B. Fluorescence immunostaining of ^{HLA-KO/CCND2-OE}hiPSC for protein expressions of Oct3/4 SOX2 and SSEA4. FIG. 21C. Hematoxylin and eosin stained teratoma section for identification of cartilage (epidermis (ectoderm), and glandular tissue endoderm (endoderm). FIG. 21D. Karyotyping of ^{HLA-KO/CCND2-OE}hiPSCs.

FIGS. 22A to 22D. Protein and gene expression of pluripotent markers in ^{HLA-KO/CCND2-OE}hiPSCs and ^{HLA-KO/CCND2-OE}hiPSC CMs Flow cytometry analysis of ^{HLA-KO/CCND2-OE} hiPSCs and ^{HLA-KO/CCND2-OE}hiPSC CMs for protein expressions of (FIG. 22A) Tra-1, (FIG. 22B) Oct3/4 and (FIG. 22C) Sox2. FIG. 22D. Gene expression of Sox2, Oct4, and Nanog in ^{HLA-KO/CCND2-OE}hiPSCs and ^{HLA-KO/CCND2-OE}hiPSC CMs.

FIGS. 23A to 23D. Increased DNA synthesis and mitosis of hiPSC CM. FIG. 23A. Co-staining of hiPSC-CMs for protein expressions of cTnT and BrdU. FIG. 23B. Quantification of BrdU+CM. FIG. 23C. Co staining of hiPSC-CMs for protein expressions of cTnT and Ki67. FIG. 23D. Quantification of Ki67+hiPSC−.

FIGS. 24A to 24D. Increased mitosis of porcine CM at the border zone of injury in the porcine heart at 1 week after I/R and ^{HLA-KO/CCND2-OE}hiPSC-CM spheroid transplantation. FIG. 24A. Co-staining of porcine heart tissues for protein expressions of Ki67 and cTnI. FIG. 24B. Quantification of Ki67+CM in the porcine hearts. FIG. 24C. Co-staining of porcine heart tissues for protein expressions of aurora B kinase and cTnI. FIG. 24D. Quantification of Aurora-B+CM in the porcine hearts.

FIGS. 25A to 25F. Quantification of porcine CM mitosis at the remote zone of injury in the porcine heart at 1 week after I/R and ^{HLA-KO/CCND2-OE}hiPSC CM spheroid transplantation. FIG. 25A. Co-staining of porcine heart tissues for protein expressions of Ki67 and cTnI. FIG. 25B. Quantification of Ki 67+CM in the porcine hearts. Figure Co-staining of porcine heart tissues for protein expressions of pH3 and cTnI. FIG. 25D. Quantification of pH3+CM in the porcine hearts. FIG. 25E. Co-staining of porcine heart tissues for protein expressions of aurora B kinase and cTnI. FIG. 25F. Quantification of Aurora-B+CM in the porcine hearts.

FIGS. 26A to 26F. Quantification of porcine CM mitosis at the border zone of injury in the porcine heart at 4 week after I/R and ^{HLA-KO/CCND2-OE}hiPSC-CM spheroid transplantation. FIG. 26A. Co-staining of porcine heart tissues for protein expressions of Ki67 and cTnI. FIG. 26B. Quantification of Ki67+CM in the porcine hearts. FIG. 26C. Co-staining of porcine heart tissues for protein expressions of pH 3 and cTnI. FIG. 26D. Quantification of pH3+CM in the porcine hearts. FIG. 26E. Co-staining of porcine heart tissues for protein expressions of aurora-B kinase and cTnI. FIG. 26F. Quantification of Aurora-B+CM in the porcine hearts. NS= No significant difference.

FIGS. 27A to 27F. Quantification of porcine CM mitosis at the remote zone of injury in the porcine heart at 4 week after I/R and ^{HLA-KO/CCND2-OE}hiPSC-CM spheroid transplantation. FIG. 27A. Co-staining of porcine heart tissues for protein expressions of Ki67 and cTnI. FIG. 27B. Quantification of Ki67+CM in the porcine hearts. FIG. 27C. Co-staining of porcine heart tissues for protein expressions of pH3 and cTnI. FIG. 27D. Quantification of pH3+CM in the porcine hearts. FIG. 27E. Co-staining of porcine heart tissues for protein expressions of aurora-B kinase and cTnI. FIG. 27F. Quantification of Aurora-B+CM in the porcine hearts. NS=No significant difference.

FIGS. 28A to 28N. Details of the cell-cycle-specific Autoencoder Batch effect was negligible as shown by high overlapping of localizations among the (FIG. 28A) SP+IR-1 week, (FIG. 28B) I/R-1 week, (FIG. 28C) SP+I/R-1 week, (FIG. 28D) I/R-4 week CMs on UMAP. Average expression of five cell-cycle markers, (FIG. 28E) AURKB, (FIG. 28F) MKI67, (FIG. 28G) INCENP, (FIG. 28H) CDCA8, and (FIG. 281) BIRC5, in cycling CM (C.CM) and non-cycling CM (Non C.CM) in the Spheroid Group at 1 week after IR and treatment. Average expression of five DNA synthesis genes, (FIG. 28J) DNA2, (MKI (FIG. 28K) POLE, (FIG. 28L) MCM6, (FIG. 28M) RRM1, and (FIG. 28N) PRIM1, in C.CM and Non C.CM in Spheroid Group at 1 week after I/R and treatment.

FIGS. 29A to 29F show Electrical Coupling between the slice and the implanted spheroids. FIG. 29A shows a dual voltage/calcium optical mapping system. Transmembrane potential (Vm) and calcium transient (CaT) indicators have the same excitation wavelength but different emission wavelengths, which are split to two cameras. One camera images slice activation and the other spheroid activation. FIG. 29B contains an image of tissue slice mapped in FIGS. 29C, 29D, and 29E (top). Dashed line shows mapped region. The focal pacing site (P), spheroid locations (1, 2, and 3), and Vm signal locations (a, b, and c) are indicated. FIG. 29C shows field pacing produced immediate and simultaneous excitation of the slice (Vm) and spheroids (CaT). FIG. 29D shows Vm and CaT activation time maps from the slice and spheroids respectively. Activation time is the steepest upslope of Vm or CaT. A wave propagates from the pacing site across the slice. CaT occurs in the spheroids shortly after they are reached by the wave. Propagation is also evident in the latency between channels in FIG. 29E. FIG. 29E shows, in some slices, 1:1 coupling exists even at 4 Hz focal pacing (top). Loss of 1:1 coupling at 3 Hz (bottom). F, Distribution of maximum pacing frequency that produced 1:1 coupling.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

### Definitions

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

By “HLA” or “human leukocyte antigen” complex is a gene complex encoding the major histocompatibility complex (MHC) proteins in humans. These cell-surface proteins that make up the HLA complex are responsible for the regulation of the immune response to antigens. In humans, there are two MHCs, class I and class II, “HLA-I” and “HLA-II”. HLA-I includes three proteins, HLA-A, HLA-B and HLA-C, which present peptides from the inside of the cell, and antigens presented by the HLA-I complex attract killer T-cells (also known as CD8+T-cells or cytotoxic T cells). The HLA-I proteins are associated with 3-2 microglobulin (B2M). HLA-II includes five proteins, HLA-DP, HLA-DM, HLA-DOB, HLA-DO and HLA-DR, which present antigens from outside the cell to T lymphocytes. This stimulates CD4+ cells (also known as T-helper cells). It should be understood that the use of either “MHC” or “HLA” is not meant to be limiting, as it depends on whether the genes are from humans (HLA) or murine (MHC). Thus, as it relates to mammalian cells, these terms may be used interchangeably herein.

By “gene knock out” herein is meant a process that renders a particular gene inactive in the host cell in which it resides, resulting either in no protein of interest being produced or an inactive form. As will be appreciated by those in the art and further described below, this can be accomplished in a number of different ways, including removing nucleic acid sequences from a gene, or interrupting the sequence with other sequences, altering the reading frame, or altering the regulatory components of the nucleic acid. For example, all or part of a coding region of the gene of interest can be removed or replaced with “nonsense” sequences, all or part of a regulatory sequence such as a promoter can be removed or replaced, translation initiation sequences can be removed or replaced, etc.

By “gene knock in” herein is meant a process that adds a genetic function to a host cell. This causes increased levels of the encoded protein. As will be appreciated by those in the art, this can be accomplished in several ways, including adding one or more additional copies of the gene to the host cell or altering a regulatory component of the endogenous gene increasing expression of the protein is made. This may be accomplished by modifying the promoter, adding a different promoter, adding an enhancer, or modifying other gene expression sequences.

iPSCs

The generation of mouse and human pluripotent stem cells (generally referred to as iPSCs; miPSCs for murine cells or hiPSCs for human cells) is generally known in the art. As will be appreciated by those in the art, there are a variety of different methods for the generation of iPCSs. The original induction was done from mouse embryonic or adult fibroblasts using the viral introduction of four transcription factors, Oct3/4, Sox2, c-Myc and Klf4; see Takahashi and Yamanaka Cell 126:663-676 (2006), hereby incorporated by reference in its entirety and specifically for the techniques outlined therein. Since then, a number of methods have been developed; see Seki et al., World J. Stem Cells 7(1):116-125 (2015) fora review, and Lakshmipathy and Vermuri, editors, Methods in Molecular Biology: Pluripotent Stem Cells, Methods and Protocols, Springer 2013, both of which are hereby expressly incorporated by reference in their entirety, and in particular for the methods for generating hiPSCs (see for example Chapter 3 of the latter reference).

Generally, iPSCs are generated by the transient expression of one or more “reprogramming factors” in the host cell, usually introduced using episomal vectors. Under these conditions, small amounts of the cells are induced to become iPSCs (in general, the efficiency of this step is low, as no selection markers are used). Once the cells are “reprogrammed”, and become pluripotent, they lose the episomal vector(s) and produce the factors using the endogeneous genes. This loss of the episomal vector(s) results in cells that are called “zero footprint” cells. This is desirable as the fewer genetic modifications (particularly in the genome of the host cell), the better. Thus, it is preferred that the resulting hiPSCs have no permanent genetic modifications.

As is also appreciated by those of skill in the art, the number of reprogramming factors that can be used or are used can vary. Commonly, when fewer reprogramming factors are used, the efficiency of the transformation of the cells to a pluripotent state goes down, as well as the “pluripotency”, e.g. fewer reprogramming factors may result in cells that are not fully pluripotent but may only be able to differentiate into fewer cell types.

In some embodiments, a single reprogramming factor, OCT4, is used. In other embodiments, two reprogramming factors, OCT4 and KLF4, are used. In other embodiments, three reprogramming factors, OCT4, KLF4 and SOX2, are used. In other embodiments, four reprogramming factors, OCT4, KLF4, SOX2 and c-Myc, are used. In other embodiments, 5, 6 or 7 reprogramming factors can be used selected from SOKMNLT; SOX2, OCT4 (POU5F1), KLF4, MYC, NANOG, LIN28, and SV40L T antigen.

In general, these reprogramming factor genes are provided on episomal vectors such as are known in the art and commercially available. For example, ThermoFisher/Invitrogen sell a sendai virus reprogramming kit for zero footprint generation of hiPSCs, see catalog number A34546. ThermoFisher also sells EBNA-based systems as well, see catalog number A14703.

In addition, there are a number of commercially available hiPSC lines available; see, e.g., the Gibco® Episomal hiPSC line, K18945, which is a zero footprint, viral-integration-free human iPSC cell line (see also Burridge et al, 2011, supra).

For example, successful iPSCs were also generated using only Oct3/4, Sox2 and Klf4, while omitting the C-Myc, although with reduced reprogramming efficiency.

In some cases, the pluripotency of the cells is measured or confirmed as outlined herein, for example by assaying for reprogramming factors as is generally shown in PCT/US18/13688 or by conducting differentiation reactions as outlined herein and in the Examples.

In some embodiments, the hiPSC comprise a “suicide gene” or “suicide switch”. These are incorporated to function as a “safety switch” that can cause the death of the pluripotent cells should they grow and divide in an undesired manner. The “suicide gene” ablation approach includes a suicide gene in a gene transfer vector encoding a protein that results in cell killing only when activated by a specific compound. A suicide gene may encode an enzyme that selectively converts a nontoxic compound into highly toxic metabolites. The result is specifically eliminating cells expressing the enzyme. In some embodiments, the suicide gene is the herpesvirus thymidine kinase (HSV-tk) gene and the trigger is ganciclovir. In other embodiments, the suicide gene is the Escherichia coli cytosine deaminase (EC-CD) gene and the trigger is 5-fluorocytosine (5-FC) (Barese et al., Mol. Therap. 20(10):1932-1943 (2012), Xu et al., Cell Res. 8:73-8 (1998), both incorporated herein by reference in their entirety.)

In other embodiments, the suicide gene is an inducible Caspase protein. An inducible Caspase protein comprises at least a portion of a Caspase protein capable of inducing apoptosis. In one embodiment, the portion of the Caspase protein is exemplified in SEQ ID NO:6. In preferred embodiments, the inducible Caspase protein is iCasp9. It comprises the sequence of the human FK506-binding protein, FKBP12, with an F36V mutation, connected through a series of amino acids to the gene encoding human caspase 9. FKBP12-F36V binds with high affinity to a small-molecule dimerizing agent, AP1903. Thus, the suicide function of iCasp9 in the instant invention is triggered by the administration of a chemical inducer of dimerization (CID). In some embodiments, the CID is the small molecule drug AP1903. Dimerization causes the rapid induction of apoptosis. (See WO2011146862; Stasi et al, N. Engl. J. Med 365; 18 (2011); Tey et al., Biol. Blood Marrow Transplant. 13:913-924 (2007), each of which are incorporated by reference herein in their entirety.)

In some embodiments, the hiPSC are derived/produced from the subject to be treated, e.g. are autologous. In other embodiments, the hiPSCs are allogeneic, e.g. are engineered according to the methods described herein.

In some embodiments, the hiPSC are derived from a single subject. In some embodiments, the hiPSC are derived from several subjects and grouped together. The hiPSC can be “healthy”, that is to say derived from a subject free from known pathology or “pathological”, e.g. derived from a subject suffering from a pathology. The subject may be affected by a cardiac pathology or any other pathology. The subject may in particular be affected by a genetic pathology, whether or not it is a cardiac pathology. By “genetic pathology” is meant here a disease, partially or completely, directly or indirectly, caused by one or more abnormalities of the genome. Said genetic pathology can in particular be a non-hereditary or hereditary pathology. By “hereditary genetic pathology” or “hereditary pathology” is meant pathologies resulting from one or more anomalies of the genome inherited from at least one parent. Preferably, said genetic pathology is a hereditary genetic pathology.

By way of non-limiting example, said subject may be suffering from a pathology such as cardiac pathology, a connective tissue disorder which can weaken the walls of the vessels (e.g. scleroderma, Ehlers-Danlos syndrome, osteogenesis imperfecta, achondroplasia, polycystic kidney disease), musculoskeletal disorder (e.g. arthritis, osteogenesis imperfecta), diabetes, glomerular disease, Duchenne muscular dystrophy and other neuromuscular disorders, Alzheimer's disease, Huntington's disease and other neuronal pathologies, cystic fibrosis, sickle cell anemia, Crohn's disease and other digestive pathologies, retinoblastoma, mitochondrial disease, etc. More generally, the subject can be affected by any disease, in particular with a genetic component. Preferably, said subject has a genetic pathology, in particular chosen from those listed here.

By way of non-limiting example, said subject may have a cardiac pathology such as cardiac amyloidosis, cardiac arrhythmia (e.g. atrial fibrillation, Brugada syndrome, catecholaminergic polymorphic ventricular tachycardia, syndrome long QT, short QT syndrome), congenital non-cyanogenic heart disease (eg leading to communication abnormalities between the heart compartments, persistent ductus arteriosus, strictures, or “stenoses”), congenital cyanogenic heart disease (leading e.g. to tetralogy of fallut or transposition of the great vessels), cardiomyopathy (e.g. dilated cardiomyopathy, hypertrophic cardiomyopathy, arrhythmogenic right ventricular cardiomyopathy, restrictive cardiomyopathy), hypercholesterolemia, atherosclerosis (e.g., coronary atherosclerosis), angina pectoris, heart defect, ischemic heart disease c chronicle, etc. The subject is preferably affected by a cardiac pathology, more preferably affected by a genetic cardiac pathology.

In some embodiments, the cardiac pathology is selected from cardiac amyloidosis, cardiac arrhythmia (e.g. atrial fibrillation, Brugada syndrome, catecholaminergic polymorphic ventricular tachycardia, long QT syndrome, short QT syndrome), congenital heart disease. non-cyanogenic (e.g. leading to communication abnormalities between cardiac compartments, persistent ductus arteriosus, strictures, or ‘stenoses’), congenital cyanogenic heart disease (e.g. leading to tetralogy or transposition of the large vessels), cardiomyopathy (e.g. dilated cardiomyopathy, hypertrophic cardiomyopathy, arrhythmogenic right ventricular cardiomyopathy, restrictive cardiomyopathy).

Engineered iPSCs

In some embodiments, e.g. where allogenic cells are being used, the iPSCs are engineered to overexpress cell-cycle regulatory gene cyclin D2 (CCND2) and/or are engineered to delete one or more human leukocyte antigen (HLA) genes.

CCND2 Overexpresion

The iPSCs can be engineered to express or overexpress a heterologous gene encoding CCND2. For example, in some embodiments, the CCND2 gene is operably linked to a myosin heavy chain (MHC) promoter.

HLA KO

In some embodiments, the pluripotent stem cells have reduced MHC I (HLA I) function and/or MHC II (HLA II) function. As will be appreciated by those in the art, the reduction in function can be accomplished in a number of ways, including removing nucleic acid sequences from a gene, interrupting the sequence with other sequences, or altering the regulatory components of the nucleic acid. For example, all or part of a coding region of the gene of interest can be removed or replaced with “nonsense” sequences, frameshift mutations can be made, all or part of a regulatory sequence such as a promoter can be removed or replaced, translation initiation sequences can be removed or replaced, etc.

As will be appreciated by those in the art, the successful reduction of the MHC (HLA) in the pluripotent cells can be measured using techniques known in the art and as described below; for example, FACS techniques using labeled antibodies that bind the HLA complex; for example, using commercially available HLA-A,B,C antibodies that bind to the alpha chain of the human major histocompatibility HLA Class I or II antigens.

The successful reduction of the MHC (HLA) function in the pluripotent cells or their derivatives can be measured using techniques known in the art such as Western blotting using antibodies to the protein, FACS techniques, RPA techniques, RT-PCR techniques, etc.

Stem Cell Differentiation

Spheroid Culture

In some embodiments, the CM spheroids, ECs, SMCs, and CFs are dissociated to produce CM, EC, SMC, and CF cell suspensions. These suspensions can be mixed at a CM:EC:SMC:CF ratio of 4:2:1:1 to produce a cardiac cell mixture. This mixture may then be cultured in microwells. The cell suspension may comprise one or more culture media suitable for culturing cardiomyocytes. Culture media for culturing cardiomyocytes are well known in the art.

The micro-wells employed in the inventive method can be any micro-wells comprising non-fouling materials known in the art that are suitable for microtissue fabrication. In some embodiments, the non-fouling materials comprise agarose or non-adhesive self-assembly plates, such as the InSphero Gravity TRAP ultra-low attachment plate. The non-fouling materials may comprise any suitable material, such as, for example, agarose gel, polyethylene glycol, alginate, hyaluronic acid, polyacryylic acid, polyacrylic amide, polyvinyl alcohol, polyhydroxyethyl methacrylate, methacrylated dextrans, poly(N-isopropylacrylamide), and any combination thereof. In some embodiments, the substrate may be any suitable unfouling hydrogel.)

In some embodiments, the cardiac cell mixture is cultured for about 1 to about 20 days, about 5 to about 15 days, or about 8 to about 12 days (e.g., about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, and any range or value herein).

In some embodiments, the cardiac cell mixture is cultured to form a self-assembled 3D cardiac organoid (spheroid) under normoxic conditions, wherein normoxic conditions comprise a partial pressure of oxygen in the gas phase of about 16% to about of the total barometric pressure (or at least about 16%, about 17%, about 18%, about 19%, or at least about 20% of the total barometric pressure).

In some embodiments, the cardiac cell mixture is cultured in the presence of an additional agent selected from norepinephrine, angiotensin II, TNF-alpha, interfering RNAs, microRNAs, matrix metalloproteases, and any combination thereof. The amount of the additional agent can vary. For example, in some embodiments, the amount of the additional agent may range from about 0.01 μM to about 10 μM, about 1 μM to about 8 μM, or from about 3 μM to about 5 μM (e.g., about 1 μM, about 2 μM, about 3 μM, about 4 μM, about 5 μM, about 6 μM, about 7 μM, about 8 μM, about 9 μM, or from about 10 μM, or any range or value therein).

A 3D cardiac organoid can be in any suitable shape. For example, in some embodiments, the 3D cardiac organoid can be in the shape of a spheroid. In some embodiments, the spheroid comprises an average diameter of about 100 to about 1000 ppm, about 200 to about 800 μm, or about 200 to about 400 μm (or of about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, or any value or range therein).

Method of Screening

In some embodiments, the disclosed 3D cardiac organoid is used in a method of screening a compound for its ability to improve or diminish cardiac function. The ability of the compound to improve or diminish cardiac function is determined by contacting the 3D cardiac organoid with a compound followed by measuring one or more characteristics of the organoid that reflect modulation of cardiac function (e.g., size of the interior apoptotic region of the 3D cardiac organoid, ratio of the TUNEL-positive area to the DAPI-positive area in the apoptotic region, contraction amplitude, beat rate, calcium transient amplitude, and/or elastic modulus). The measurements of these characteristics can then be compared with corresponding reference values for a 3D cardiac organoid that has not been contacted with the compound, thereby determining the effect(s) of the compound on one or more of the measured characteristics that reflect cardiac function. The compound can be any compound of interest, such as, for example, a therapeutic compound. Exemplary therapeutic compounds include, but are not limited to, a therapeutic compound for treating cardiovascular disease, diabetes, kidney disease, liver disease, and/or cancer. In some embodiments, the compound is a small-molecule, nucleic-acid based drug and/or protein-based drug.

In some embodiments, a method of screening a compound for improving cardiac function may comprise contacting the 3D cardiac organoid with the compound and measuring in the 3D cardiac organoid one or more of: (a) size of the interior apoptotic region, (b) ratio of a TUNEL-positive area to a DAPI-positive area in the apoptotic region, (c) contraction amplitude, (d) beat rate, (e) calcium transient amplitude, and/or (0 elastic modulus.

In some embodiments, a compound may be determined to improve cardiac function when the size of the interior apoptotic region is reduced by at least about 30%, about 40%, about 50%, about 60%, about 70%, about 80%, about 90%, or about 100% when compared to a control 3D myocardial infarct organoid and/or a 3D myocardial ischemia-reperfused organoid that has not been contacted with the compound.

In some embodiments, a compound may be determined to improve cardiac function when the ratio of TUNEL-positive area to DAPI-positive area is reduced by at least about 30%, about 40%, about 50%, about 60%, about 70%, about 80%, about 90%, or about 100% when compared to a control 3D cardiac organoid that has not been contacted with the compound. This ratio can vary but typically ranges from about 0.03 to about 1 in a control 3D cardiac organoid that has not been contacted with the compound.

In some embodiments, a compound may be determined to improve cardiac function when the contraction amplitude is increased by at least about 30%, about 40%, about 50%, about 60%, about 70%, about 80%, about 90%, or about 100% when compared to a control 3D cardiac organoid that has not contacted with the compound. This contraction amplitude can vary but typically ranges from about 0% to about 5% in a control 3D cardiac organoid that has not contacted with the compound.

In some embodiments, a compound may be determined to improve cardiac function when the calcium transient amplitude is increased by at least about 30%, about 40%, about 50%, about 60%, about 70%, about 80%, about 90%, or about 100% when compared to a control 3D cardiac organoid that has not been contacted with the compound. This calcium transient amplitude can vary but typically ranges from about 0% to about 40% in a control 3D cardiac organoid that has not been contacted with the compound.

In some embodiments, a compound may be determined to improve cardiac function when the elastic modulus is decreased by at least about 30%, about 40%, about 50%, about 60%, about 70%, about 80%, about 90%, or about 100% when compared to a control 3D cardiac organoid that has not been contacted with the compound. The elastic modules of a control 3D cardiac organoid can vary but typically ranges from about 3 kPa to about 5 kPa.

Another aspect of the invention relates to a method for screening a compound for diminishing cardiac function. For example, such compounds include therapeutic compounds used for treating diseases other than cardiovascular diseases. Screening of any cardiovascular effects of such compounds in a 3D cardiac organoid provides useful information as to the potential cardiotoxicity associated with these compounds when administered to a mammals (e.g., a human) that is already cardio-compromised (i.e., wherein the heart is not functioning at full capacity). In some embodiments, the method may comprise contacting the 3D cardiac organoid with a test compound and measuring one or more of: (a) size of the interior apoptotic region, (b) ratio of a TUNEL-positive area to a DAPI-positive area in the apoptotic region, (c) contraction amplitude, (d) beat rate, (e) calcium transient amplitude, and/or (f) elastic modulus.

In some embodiments, a compound may be contact with a population of 3D cardiac organoids. In some embodiments, a population of 3D cardiac organoids may comprise about 2 to about 100, about 2 to about 80, about 2 to about 70, about 2 to about 50, about 2 to about 40, about 2 to about 35, about 2 to about 25 or about 2 to about 10 3D cardiac organoids. In some embodiments, the number (percentage of the total population) of asynchronously beating 3D cardiac organoids in the population may be determined after contacting the population with a test compound. In some embodiments, a compound may be determined to improve cardiac function when the percentage of asynchronously-beating organoids in the population (e.g., an asynchronously-beating subpopulation) is decreased by more than about 30%, about 40%, about 50% about 60%, about 70%, about 80%, about 90%, or about 100% when compared to a control 3D cardiac organoid that has not been contacted with the compound. In a control population of 3D cardiac organoids that have not been contacted with the compound, the percentage of organoids that make up the asynchronously-beating subpopulation may vary but typically ranges from about 30% to about 100%.

In some embodiments, a compound may be determined to diminish cardiac function when the size of the interior apoptotic region is increased by at least about 30% about 40% about 50%, about 60%, about 70%, about 80%, about 90%, or about 100% when compared to a control 3D myocardial infarct organoid and/or control 3D myocardial ischemia-reperfused organoid that is not contacted with the compound. This size of the apoptotic region can vary but typically ranges from about 20 μm to about 75 μm in a control 3D myocardial infarct organoid and/or a control 3D myocardial ischemia-reperfused organoid that has not been contacted with the compound.

In some embodiments, a compound may be determined to diminish cardiac function when the ratio of TUNEL-positive area to DAPI-positive area is increased by at least 30%, about 40%, about 50%, about 60%, about 70%, about 80%, about 90%, or about 100% when compared to a control 3D cardiac organoid that has not been contacted with the compound. This ratio can vary but typically ranges from about 0.03 to about 1 in a control 3D cardiac organoid that has not been contacted with the compound.

In some embodiments, a compound may be determined to diminish cardiac function when the contraction amplitude is decreased by at least about 30%, about 40%, about 50%, about 60%, about 70%, about 80%, about 90%, or about 100% when compared to a control 3D cardiac organoid that has not been contacted with the compound. This contraction amplitude can vary but typically ranges from about 0% to about 5% in a control 3D cardiac organoid that has not been contacted with the compound.

In some embodiments, a compound may be determined to diminish cardiac function when the calcium transient amplitude is decreased by at least about 30%, about 40%, about 50%, about 60%, about 70%, about 80%, about 90%, or about 100% when compared to a control 3D myocardial infarct organoid and/or control 3D cardiac organoid that has not been contacted with the compound. This calcium transient amplitude can vary but typically range from about 0% to about 40% in a control 3D cardiac organoid that has not been contacted with the compound.

In some embodiments, a compound may be determined to diminish cardiac function when the elastic modulus is increased by about 30%, about 40%, about 50%, about 60%, about 70% about 80%, about 90%, or about 100% when compared to a control 3D cardiac organoid that has not contacted with the compound. This elastic modulus of a control 3D cardiac organoid can vary but typically ranges from about 3 kPa to about 5 kPa.

Methods of Treatment

In some embodiments, the cardiac spheroids disclosed herein are administered to a patient, e.g., a human patient in need thereof. The cardiac spheroids can be administered to a patient suffering from pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, other cardiomyopathy, myocarditis, myocardial ischemic reperfusion injury, ventricular dysfunction, heart failure, congestive heart failure, coronary artery disease, end stage heart disease, atherosclerosis, ischemia, hypertension, restenosis, angina pectoris, rheumatic heart, arterial inflammation, or cardiovascular disease. In some instances, the patient has had a myocardial infarction. In particular instances, the patient is undergoing coronary artery bypass surgery.

The cardiac spheroids can be transplanted into the patient using well known surgical techniques for grafting tissue and/or isolated cells into a heart. In some embodiments, the cells are introduced into the patient's heart tissue by injection (e.g., intramyocardial injection, intracoronary injection, trans-endocardial injection, trans-epicardial injection, percutaneous injection), infusion, and implantation.

Administration (delivery) of the cardiac spheroids include, but are not limited to, subcutaneous or parenteral including intravenous, intraarterial (e.g. intracoronary), intramuscular, intraperitoneal, intramyocardial, trans-endocardial, trans-epicardial, intranasal administration as well as intrathecal, and infusion techniques.

In some embodiments, the patient administered the cardiac spheroids is also administered a cardiac drug. Illustrative examples of cardiac drugs that are suitable for use in combination therapy include, but are not limited to, growth factors, polynucleotides encoding growth factors, angiogenic agents, calcium channel blockers, antihypertensive agents, antimitotic agents, inotropic agents, anti-atherogenic agents, anti-coagulants, beta-blockers, anti-arhythmic agents, anti-inflammatory agents, vasodilators, thrombolytic agents, cardiac glycosides, antibiotics, antiviral agents, antifungal agents, agents that inhibit protozoans, nitrates, angiotensin converting enzyme (ACE) inhibitors, angiotensin II receptor antagonist, brain natriuretic peptide (BNP); antineoplastic agents, steroids, and the like.

The effects of therapy according to the methods of the invention can be monitored in a variety of ways. For instance, an electrocardiogram (ECG) or holter monitor can be utilized to determine the efficacy of treatment. An ECG is a measure of the heart rhythms and electrical impulses, and is a very effective and non-invasive way to determine if therapy has improved or maintained, prevented, or slowed degradation of the electrical conduction in a subject's heart. The use of a holter monitor, a portable ECG that can be worn for long periods of time to monitor heart abnormalities, arrhythmia disorders, and the like, is also a reliable method to assess the effectiveness of therapy. An ECG or nuclear study can be used to determine improvement in ventricular function.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: A Three-Dimensional Culture System for Generating Cardiac Spheroids Composed of Cardiomyocytes, Endothelial Cells, Smooth-Muscle Cells, and Cardiac Fibroblasts Derived from Human Induced-Pluripotent Stem Cells

Abstract

Cardiomyocytes (CMs), endothelial cells (ECs), smooth muscle cells (SMCs), and cardiac fibroblasts (CFs) differentiated from human induced-pluripotent stem cells (hiPSCs) are the fundamental components of cell-based regenerative myocardial therapy and can be used as in-vitro models for mechanistic studies and drug testing. However, newly differentiated hiPSC-CMs tend to more closely resemble fetal CMs than the mature CMs of adult hearts, and current techniques for improving CM maturation can be both complex and labor-intensive. Thus, the production of CMs for commercial and industrial applications will require more elementary methods for promoting CM maturity. CMs tend to develop a more mature phenotype when cultured as spheroids in a three-dimensional (3D) environment, rather than as two-dimensional monolayers, and the activity of ECs, SMCs, and CFs promote both CM maturation and electrical activity. Here, we introduce a simple and reproducible 3D-culture-based process for generating spheroids containing all four cardiac-cell types (i.e., cardiac spheroids) that is compatible with a wide range of applications and research equipment. Subsequent experiments demonstrated that the inclusion of vascular cells and CFs was associated with an increase in spheroid size, a decline in apoptosis, an improvement in sarcomere maturation and a change in CM bioenergetics.

### INTRODUCTION

The development of efficient protocols for differentiating human induced-pluripotent stem cells (hiPSCs) into cardiomyocytes (CMs), endothelial cells (ECs), smooth muscle cells (SMCs), and cardiac fibroblasts (CFs) (Adams et al., 2013; Liu et al., 2016; Palpant et al., 2017; Zhu et al., 2017a; Kwong et al., 2019; Zhang et al., 2019; Kahn-Krell et al., 2021) has led to the establishment of an array of utilizations in the field of cardiac regeneration. Although early proposed applications focused on direct injections (Menasche et al., 2001; Laflamme et al., 2007; Sanganalmath and Bolli, 2013; Ye et al., 2014) and the transplantation of engineered cardiac tissues (Schaefer et al., 2017; Shadrin et al., 2017; Gao et al., 2018) and sheets (Ishigami et al., 2018; Bayrak and Gümü§ derelio\check{g}lu, 2019; Tsuruyama et al., 2019) to resupply cardiomyocytes, lost to injuries such as myocardial infarction (MI), the uses of these cardiac surrogates have expanded to include in-vitro modeling of myocardial disease (Long et al., 2018; Giacomelli et al., 2021), drug screening (Mathur et al., 2015; Huebsch et al., 2016; Mills et al., 2017), and exosome production (Liu et al., 2017). A wide range of formats has been explored to fulfill the needs of each potential application including patches (Schaefer et al., 2017; Shadrin et al., 2017; Gao et al., 2018), sheets (Ishigami et al., 2018; Bayrak and Gümü§ derelio\check{g}lu, 2019; Tsuruyama et al., 2019), spheres (Fischer et al., 2018; Chang et al., 2020), wires (Jackman et al., 2016; Sun and Nunes, 2016), and decellularized tissues (Das et al., 2019; Alexanian et al., 2020; Hochman-Mendez et al., 2020). The utilization of these products and the needs of each use play a key role in the ideal option for each situation. However, there is a crucial need to establish tools for multiple applications related to MI and heart failure that can be easily reproduced and scaled (Zhang et al., 2021a).

Although current differentiation techniques can produce beating hiPSC-derived CMs in as little as 9 days (Lian et al., 2012; Zhang et al., 2012), the biomolecular (Cao et al., 2008; Synnergren et al., 2008; Xu et al., 2009; Synnergren et al., 2010), electrical (Caspi et al., 2009; Kim et al., 2010; Lee et al., 2011), and mechanical (Binah et al., 2007; Zhang et al., 2021b) properties of these newly differentiated cells tend to resemble those of fetal CMs, rather than the mature CMs of adult hearts (Robertson et al., 2013). Electrical stimulation (Chan et al., 2013; Richards et al., 2016; Ruan et al., 2016; LaBarge et al., 2019a) and mechanical stretching (Lux et al., 2016; Ruan et al., 2016; Zhang et al., 2017; LaBarge et al., 2019a) have been used to increase the maturity of engineered cardiac tissue but are difficult to apply until after the tissue is manufactured. Thus, the production of CMs for commercial and industrial applications will require more elementary methods for promoting CM maturation (Zhang et al., 2021a), such as manipulating the conditions of hiPSC-CM culture (Lin et al., 2017a; Parikh et al., 2017; Jackman et al., 2018; Selvaraj et al., 2019).

Another method to increase complexity and functionality of cardiac tissue models is to use additional cell types that more accurately recreate the myocardial environment. When cultured in a three dimensional (3D) environment, rather than as two-dimensional monolayers, CMs coalesce into spheroids (Fischer et al., 2018; Chang et al., 2020) and tend to develop a more mature phenotype (Jha et al., 2016; Sacchetto et al., 2020; Wang et al., 2021a). ECs and SMCs also promote CM maturation (Pinto et al., 2016; Ayoubi et al., 2017; Giacomelli et al., 2017; Zhu et al., 2017b; Kwong et al., 2019) while facilitating oxygen and nutrient delivery, which improves cell survival (Garzoni et al., 2009; Pretorius et al., 2021; Zhang et al., 2021a), and CFs contribute to myocardial development both by producing components of the extracellular matrix (ECM) and by forming gap junctions with CMs to support electronic signal transduction (Zhang et al., 2019; Beauchamp e 77 t al., 2020; Giacomelli et al., 2020; Li et al., 2020; Pretorius et al., 2021). A range of ratios between these different cell types have been explored in previous studies but a relationship of CM:EC:SMC:CF of 4:2:1:1 is predominantly used (Gao et al., 2018; Arai et al., 2020; Beauchamp et al., 2020; Daly et al., 2021; Pretorius et al., 2021) as it roughly recapitulates the relationships found in native myocardium of myocytes predominating with endothelial cells compromising the greatest non-myocyte population (Banerjee et al., 2007; Pinto et al., 2016).

Along with the functionality and maturity requirements for broad cardiac tissue surrogate application two important biomanufacturing considerations are system format and scalability. A uniform shape, size, and culture condition that can function in a range of uses would allow for centralized production (Abbasalizadeh et al., 2017; Adil and Schaffer, 2017; Li et al., 2017; Tomov et al., 2019). Spherical spheroids cultured in suspension provide a format that is broadly compatible with existing research equipment, is highly movable, and can function as a building block for larger construct needs (Mattapally et al., 2018; LaBarge et al., 2019a; LaBarge et al., 2019b; Kim et al., 2020; Daly et al., 2021; Polonchuk et al., 2021). Additionally, using previously established CM spheroid production processes combined with whole spheroid fusion, a scalable spheroid biomanufacturing platform can be established that does not require dissociation (Beauchamp et al., 2015; Lin et al., 2017b; Miwa et al., 2020; Kahn-Krell et al., 2021).

For the experiments described in this report, we differentiated hiPSCs into CMs, ECs, SMCs, and CFs and then combined the differentiated cells in a 3D culture environment, where they formed spheroids containing all four cardiac-cell types (i.e., cardiac microtissues). Subsequent analyses suggested that the inclusion of vascular cells and CFs increased spheroid size, reduced cellular apoptosis, and tended to promote sarcomere maturation and CM energy production. This process, with the potential to be scaled, advances on previous cardiac spheroid models (Giacomelli et al., 2017; Voges et al., 2017; Helms et al., 2019; Keung et al., 2019; Lee et al., 2019b; Beauchamp et al., 2020; Buono et al., 2020; Giacomelli et al., 2020; Israeli et al., 2020; Kupfer et al., 2020; Thomas et al., 2021) by producing large diameter tissues with cells from a single iPSC line, no exogenous matrix, and a reproducible product.

Materials and Methods

hiPSC Culture and Expansion

All cells used in the study were differentiated from human induced pluripotent stem cell (hiPSC) line LZ-hiPSC5 which was reprogramed from human cardiac fibroblasts as described previously (Zhang et al., 2014). hiPSCs were cultured on 6-well plates in mTeSR Plus (STEMCELL Technologies) for 3 days, detached with gentle cell dissociation reagent (GCDR) (STEMCELL Technologies), resuspended in 40 mL TeSR E8 3D seed media (STEMCELL Technologies) supplemented with 10 μM Y27632 at a density of 1.5×10⁵ cells/mL, and then cultured in a 125 mL Erlenmeyer flask on a Belly Dancer orbital shaker (IBI Scientific) at a speed of 4.75. On each of the following 2 days, 1.2 mL of feed medium was added to the flask, and after an additional day, half of the culture volume was replaced with fresh seed medium. After four days of culture on the orbital shaker, aggregates were dissociated with GCDR for 8 minutes, broken into smaller cell clumps via pipetting through a 37-μm reversible strainer (STEMCELL Technologies), and then diluted to a density of 1.5×10⁵ cells/mL. Cells were cultured as previously described (Kahn-Krell et al., 2021) for four more days before differentiation was initiated.

CM Differentiation

Cardiomyocyte (CM) differentiation was performed as described previously (Kahn-Krell et al., 2021). Briefly, differentiation was initiated on differentiation day (dD) 0 by replacing the culture media with RPMI 1640 supplemented with 1×B27 without insulin (RPMI/B27−), 6 μM CHIR99021, and 10 μM Y-27632 at a density of 1.5×106 cells/mL. On dD1, the media was replaced with a 1.2-fold volume of RPM I/B27—, 1 μM CHIR99021, and 10 μM Y-27632, and the cells were cultured for two more days. On dD3, 70% of the culture media was replaced with RPMI/B27− supplemented with 10 μM IWR-1-endo; on dD5, the media was changed to RPM I/B27−; and on dD7, the media was changed to RPM11640 supplemented with B27 with insulin (RPMI/B27+) in a volume equivalent to the volume used on dD0. Metabolic purification of the differentiated CMs was initiated on dD9 by changing the media to RPMI1640 without glucose supplemented with B27+ and 0.12% (w/w) sodium DL-lactate (Millipore Sigma). Three days later (on dD12), the purification media was replaced with RPMI/B27+, and the medium was refreshed every 5 days until spheroid assembly. Spheroid assembly was performed no more than 30 days after differentiation was initiated and as close to dD12 as possible.

EC Differentiation

Endothelial-cell (EC) differentiation of hiPSCs was performed in monolayers of cultured hiPSCs with the STEMdiff Endothelial Differentiation Kit (STEMCELL Technologies) as directed by the manufacturer's instructions. Briefly, hiPSCs were seeded into mTeSR Plus (STEMCELL Technologies) in a 6 well plate at a density of 5.0×104 per well and then on dD1 and dD2, the medium was changed to 3 mL STEMdiff Mesoderm Induction Medium (STEMCELL Technologies). The medium was replaced with 4 mL of STEMdiff Endothelial Induction Medium (STEMCELL Technologies) on dD3 and refreshed on dD5. On dD7, the cells were dissociated with ACCUTASE (Corning), transferred into a fibronectin-coated T75 flask, and cultured in EGM-2 MV (Lonza) supplemented with SB431542 (Fischer Scientific) until 100% confluent. Purification was performed by dissociating the cells, resuspending them in cold Dulbecco phosphate-buffered saline (DPBS) with 2% fetal bovine serum (FBS) at a density of 1×10⁶ cells/100 μL, and then collecting cells that expressed both CD31 and CD144 via flow cytometry on a BD FACS Aria 11 instrument; 100-μL samples were labeled by incubating them with 5 μL of AlexaFluor-conjugated CD31 and 20 μL of phycoerythrin-conjugated CD144 antibodies for 45 minutes on ice and then washed in DPBS.

SMC Differentiation

hiPSCs were treated via the EC differentiation protocol through dD7 and then differentiated into smooth-muscle cells (SMCs) as described previously (Yang et al., 2016). Briefly, the cells were cultured until 80% confluent, and then the media was changed to high-glucose DMEM supplemented with 5% FBS, 5 ng/mL platelet-derived growth factor beta (PDGF-3), and 2.5 ng/mL transforming growth factor beta (TG93). The media was changed every 3 days for 9 days and then replaced with SmGM-2.

Cardiac Fibroblast Differentiation

When hiPSCs reached 100% confluency the medium was changed to RPMI/B27− insulin supplemented with 12 μM CHIR99021 (Tocris) for 24 hours, RPMI/B27− insulin for 24 hours, and then cardiac fibroblast differentiation basal (CFBM) medium (DMEM, high glucose with HAS, linoleic acid, lecithin, ascorbic acid, GlutaMAX, hydrocortisone hemisuccinate, rh insulin) supplemented with 75 ng/ml bFGF (WiCell Research Ins 162 titute) for 18 days; the CFBM/bFGF 163 medium was changed every other day.

CM, EC, SMC, and CF Purity Assessments

Cells were dissociated, washed in DPBS, fixed with 4% paraformaldehyde (PFA) for 15 minutes, washed three times, permeabilized with 0.1% Triton-X in DPBS, and blocked with 4% bovine serum albumin (BSA) and 4% FBS in DPBS for 30 minutes each; then, the cells were incubated with lineage-specific antibodies (Table 2) for 1 hour and analyzed on an Attune NxT flow cytometer (Thermo Fisher Scientific).

Spheroid Fabrication and Culture

A 2 mL sample of CM spheroids were treated with CM dissociation media (CMDM) (STEMCELL Technologies) for 15 minutes at 37° C. with periodic mixing then counted to determine culture density. ECs, SMCs, and CFs were dissociated from monolayer cultures and 5.0×10⁶, 2.5×10⁶, and 2.5×10⁶ of each respectively were combined and resuspended in 1 mL of spheroid media (OM) (Table 4). Based on cell counts 1.0×10⁷ total CMs were collected as whole spheroids in a 50 mL conical tube and the media was replaced with 19 mL OM. After addition of the other cell types the contents were mixed well and 200 μL was transferred to each well of a 96-Well, Nunclon Sphera-Treated, U-Shaped-Bottom Microplate (Thermo Fisher Scientific) to produce 96 spheroids. The media was refreshed every 2 days for 7 days, and then the spheroids were transferred to 6-well Ultra-Low Attachment Microplates (Corning), with no more than 16 spheroids per well. Plates were maintained on a belly dancer shaker (IBI Scientific) at speed of 4.75, and the media was exchanged every 4-7 days.

Spheroid Size Measurements

Spheroids were photographed with an Olympus CKX53 microscope at 4× magnification, and spheroid diameters were determined with a modified ImageJ macro (Ivanov et al., 2014).

Quantitative Polymerase Chain Reaction (qPCR)

RNA was extracted with TRIZOL (Thermo Fisher) and purified on Direct-zol RNA Miniprep Plus (Zymo Research) columns as directed by the manufacturer's protocol. Reverse transcription was performed with Superscript IV VI LO Master Mix (Invitrogen), and samples were diluted to 5 ng/μL. Each qPCR reaction was performed with 5 ng of cDNA, 0.5 μM primers (Table 3), and PowerUp SYBR Green Master Mix (Applied Biosystems), and analysis was conducted on a QuantStudio Real-Time PCR Machine (Applied Biosystems). Results for each CT value were normalized to intrinsic glyceraldehyde phosphate dehydrogenase (GAPDH) abundance and to the CT value determined on the first day after spheroid assembly for each batch of spheroids. Data was collected from four independent batches of spheroids for each gene.

Western Blotting

Spheroids were digested in 100 μL RIPA Buffer (Sigma-Aldrich); then, protein lysates were collected, mixed with 1× Halt proteinase inhibitor cocktail mix (Thermo Scientific), and sonicated twice in 3-second intervals at 50% power. Sample concentrations were determined via Pierce BCA 21 Protein Assay (Thermo Scientific), and then samples containing 4× Laemmli Sample Buffer (Bio-Rad), 2 Mercaptoethanol (Bio-Rad), and 7.5 μg protein were run on an Expressplus PAGE 4-20% Gel (GenScript) at 200 V for 30 minutes. Proteins were transferred to nitrocellulose membranes with a Trans-Blot Turbo Transfer System (Bio-Rad); then, the membranes were blocked in 5% Blotting Grade Blocker Non Fat Dry Milk (Bio-Rad) for 30 minutes and stained with primary and secondary antibodies (Table 2) for 1 hour each. Blots were incubated for 5 minutes in Pierce SuperSignal West Pico PLUS Chemiluminescent Substrate (Thermo Scientific) and imaged with the ChemiDoc Touch Imaging System (Biorad). Protein bands were quantified with Image Lab Software (Bio-Rad); results for each sample were normalized to intrinsic beta actin abundance and to measurements made on the first day after spheroid assembly for each batch of spheroids. Data was collected from four independent batches of spheroids for each protein.

Spheroid Preservation and Sectioning

Spheroids were fixed in 4% formaldehyde (Pierce) for 15 minutes, placed in 30% sucrose at 4° C. overnight, and then then embedded in Tissue-Plus O.C.T Compound (Fisher Scientific) for histological analysis. Blocks were cut into 10-μm sections, mounted on charged glass microscope slides (Globe Scientific) and then stored at −20° C.

Masson-Trichrome Staining

Slides containing sections obtained at 100-μm intervals (i.e., every tenth section) were fixed in Bouin's Solution at 55° C. for 1 hour and then treated with Weigerts iron hematoxylin working solution for 10 minutes, with Biebrich scarlet-acid fuchsin for 5 minutes, with phosphomolybdic acid-phosphotungstic acid for 5 minutes, with aniline blue solution for 5 minutes, and with 1% acetic acid for 1 minute; then, the sections were dehydrated in 95% alcohol for 2 minutes, cleared with 2 changes of xylene, and mounted with permount and coverslips overnight. Sections were imaged with a 10× objective on an Olympus BX51 Fluorescence Microscope; when multiple fields of view were required for a single section, the images were stitched together with Photoshop software.

Apoptosis

The spheroid core was identified by determining which Masson-trichrome-stained sections had the greatest surface area; then, sections from the core were stained using the In Situ Cell Death Detection Kit, TMR red (Sigma) according to the manufacturers protocol. Briefly, sections were fixed in 4% PFA for 15 minutes, permeabilized in 0.1% Triton X-100 and 0.1% sodium citrate for 2 min on ice, incubated with TUNEL reaction mixture and 4′, 6-diamidino-2-phenylindole (DAPI) for 1 hour at 37° C., mounted in VECTASHIELD hardset Antifade Mounting Medium, and visualized via confocal laser scanning (Olympus FV3000 confocal microscope). Sections were evaluated for at least two spheroids in each of four batches, and positively stained cells or the size of stained regions were quantified with a modified ImageJ macro (Ivanov et al., 2014).

Immunostaining and Apoptosis Detection

The spheroid core was identified by determining which Masson-trichrome-stained sections had the greatest surface area; then, sections from the core were fixed in 4% PFA for 15 minutes, blocked, permeabilized in 10% donkey serum, 10% Tween20, 3% BSA, and 0.05% Triton-X for 30 minutes, incubated with primary antibodies (Table 2) at room temperature for 45 minutes, washed with PBS (3 washes, 5 minutes per wash), incubated with DAPI and fluorescent secondary antibodies at room temperature for 45 minutes, mounted in VECTASHIELD hardset Antifade Mounting Medium, and visualized via confocal laser 244 scanning (Olympus FV3000 confocal microscope). Sections were evaluated for at least two spheroids in each of four batches, and positively stained cells or the size of stained regions were quantified with a modified ImageJ macro (Ivanov et al., 2014) as indicated in the Supplemental Information.

ATP, NAD, and NADH Quantification

ATP content was measured using a luminescent ATP detection Assay kit (ab113849; Abcam) following the manufacturer's protocol. The amount of ATP was normalized to the total protein content, which was determined using a protein assay (23227, ThermoFisher). The data was presented as nanomoles per milligram protein. Total NAD+ and NADH levels were measured by colorimetric kit (ab65348; Abcam) according to manufacturer's instruction. cAMP level was measured by Direct cAMP Enzyme-linked Immunosorbent Assay (ELISA) using a direct cAMP ELISA kit (ADI-900-066A, Enzo Life Sciences) following the manufacturer's instructions. Tissue cAMP level was normalized to the total protein content and presented as picomole per milligram protein.

Microelectrode Array (MEA)

CytoView MEA 24 well plates (Axion Biosystems) were precoated with 3 μg/mL fibronectin and incubated at 37° C. for 1 hour. Whole spheroids (1 per well) or CMs collected from dissociated spheroids were added to each well of the MEA plate. Isolated CMs were obtained by treating spheroids with CMDM and periodic pipetting for 30 minutes at 37° C., selected with the EasySep Human PSC-Derived Cardiomyocyte Enrichment Kit (StemCell), and resuspended at a concentration of 1.2×10⁷ cells/mL in CM support media (CMSM) (StemCell Technologies); 5 μL of resuspended CMs were added to each well. Whole spheroids were analyzed 24-48 hours after plating, and CMs were analyzed 7 days after plating. Field potential and contractility measurements were collected on a Maestro Edge apparatus (Axion Biosystems) and analyzed by using the Cardiac Module in Axion Navigator software. Action potential durations (APDs) were determined using the LEAP assay and characterized using the Cardiac Analysis Tool.

Transmission Electron Microscopy (TEM)

Spheroids were transferred onto a fibronectin-coated (1 μg/mL), 0.4-μm pore Transwell Polycarbonate Membrane and cultured for 3 days; then, the membranes were fixed in 2.5% glutaraldehyde solution for 1 hour at 4° C. and delivered to the UAB High-Resolution Imaging Facility. Sample blocks were sectioned along the width of the transwells with a diamond knife, and samples were mounted and viewed with a Tecnai Spirit T12 Transmission Electron Microscope. At least 4 images were collected for each spheroid, and sarcomere lengths and widths were determined for all sarcomeres in an image by using the line-measure tool in ImageJ software. Sarcomere lengths were determined as the distance from z-line to z-line and widths were determined as the distance from one end to the other of a continuous z-line.

Statistical Analysis

Data are presented as mean±SEM, as box-and-whisker plots, or as violin plots, and significance was evaluated via the Student's t-test or analysis of variance (ANOVA). Analyses were performed with GraphPad Prism8 software (GraphPad Prism, RRID:SCR_002798), and p<0.05 was considered significant.

Results

The Inclusion of hiPSC-ECs, -SMCs, and CFs in CM Spheroids Increased Spheroid Size and Improved Cell Viability.

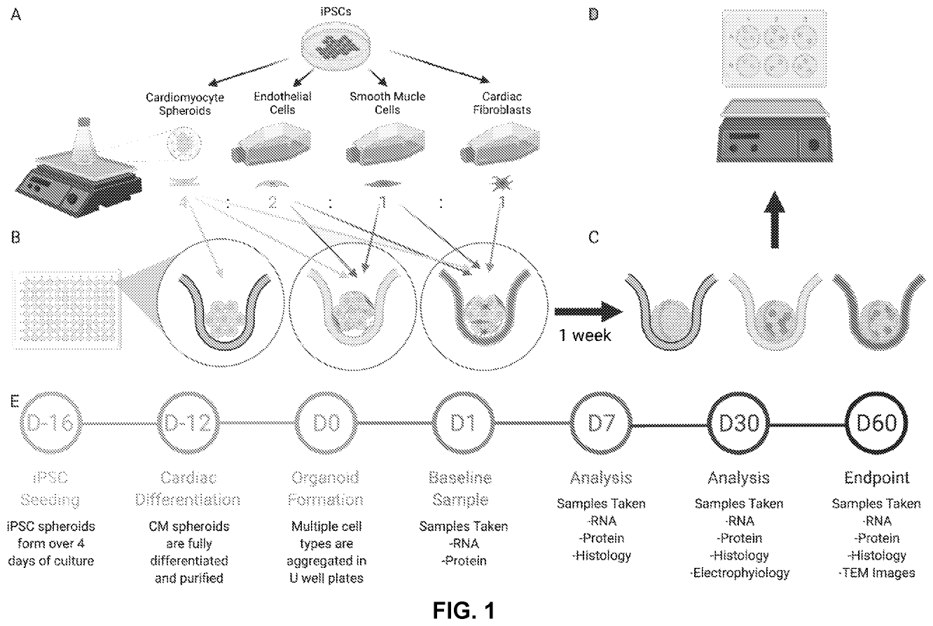

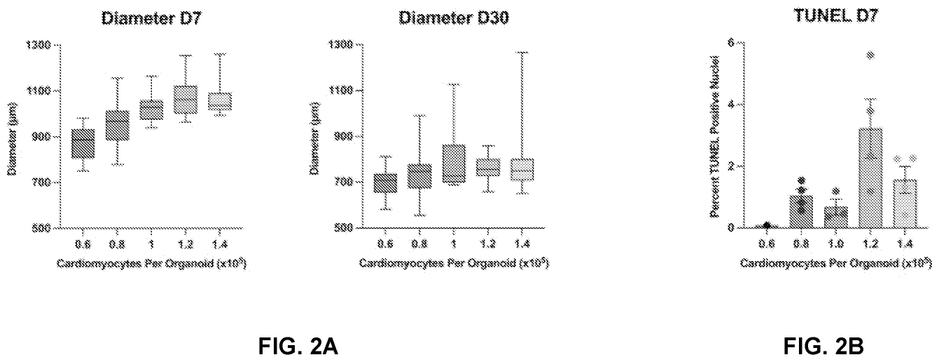

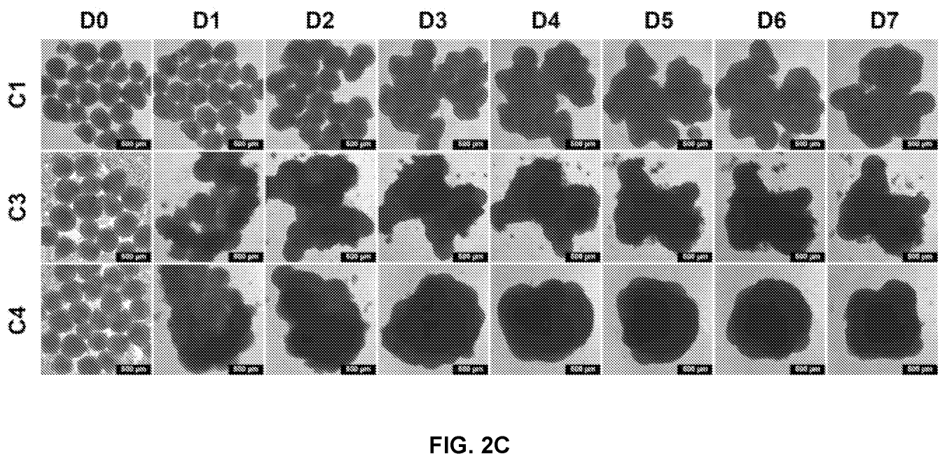

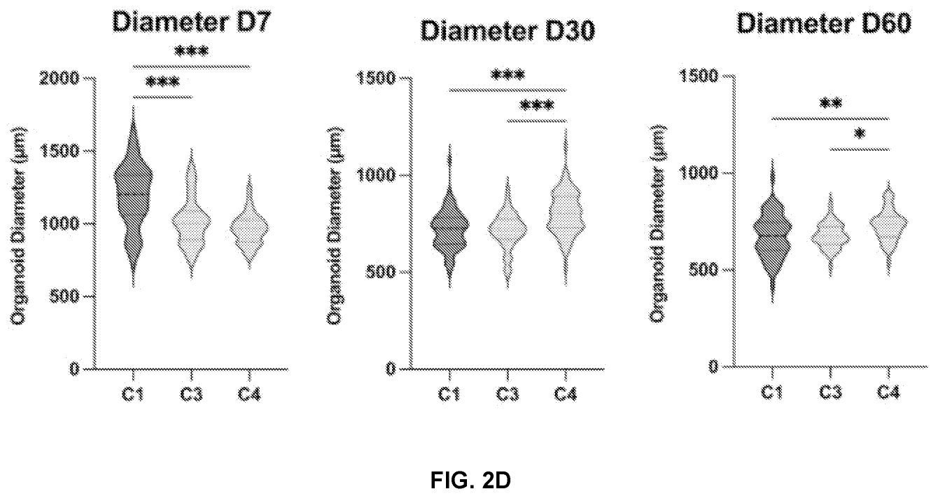

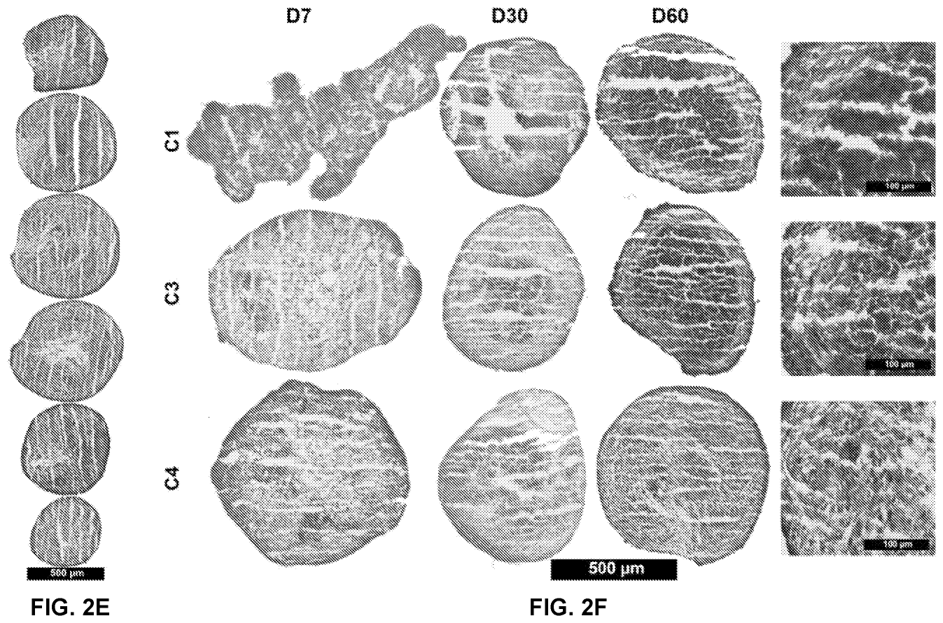

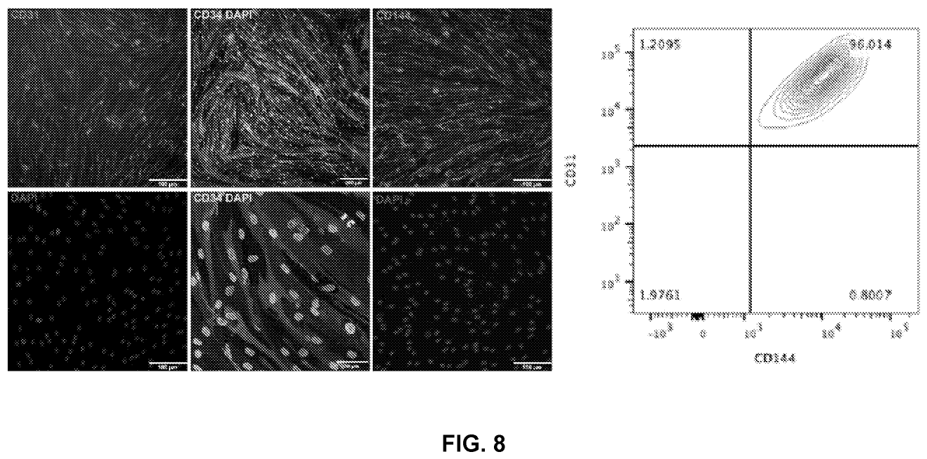

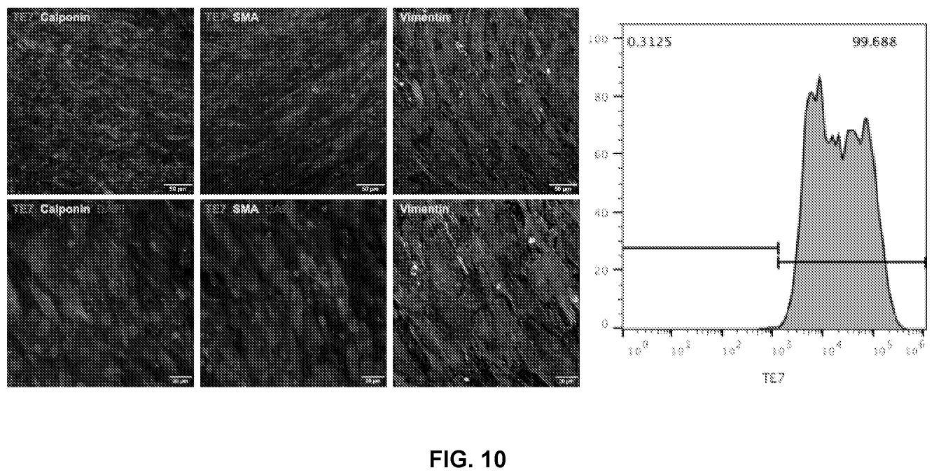

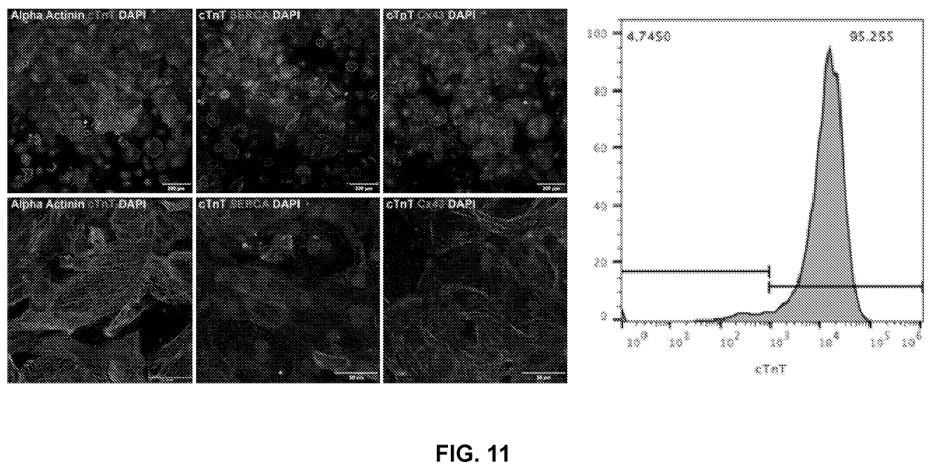

hiPSCs were differentiated into CMs, ECs, SMCs, and CFs via published protocols or the use of commercially available kits (Yang et al., 2016; Zhang et al., 2019; Kahn-Krell et al., 2021), and flow cytometry analyses with lineage-specific antibodies (CMs: cardiac troponin T [cTnT], ECs: CD144 and CD31, SMCs: smooth-muscle actin [SMA], CFs: TE-7) confirmed that the purity of each differentiated cell population exceeded 95% (FIGS. 8-11). Spheroids containing CMs alone (C1) and spheroids containing a 4:2:1 ratio of CMs, ECs, and SMCs (C3) or a 4:2:1:1 ratio of CMs, ECs, SMCs, and CFs (C4) were produced by culturing the indicated proportions of cell types in low-attachment 96-well U plates for one week to promote aggregation and fusion (FIGS. 1A-1B), and then in low-attachment 6-well plates on an orbital shaker for the remainder of the culture period (FIGS. 1C-1D). DO was defined as the day the spheroids/spheroids were initially assembled, and samples were obtained after one (D1), seven (D7), 14 (D14), 30 (D30), and 60 (D60) days of culture for characterization (FIG. 1E); synchronized beating was observed in all constructs throughout the culture period. Size optimization studies determined that the mean diameter of C4 spheroids on D30 was larger (though not significantly) when the spheroids were generated from 1.0×10⁵ seeded CMs (˜801 μm) than from other initial CM population sizes (698-791 μm) (FIG. 2A), with no significant increase in the proportion of apoptotic cells (FIG. 2B). Thus, all spheroids and spheroids produced for subsequent experiments were constructed with 1.0×10⁵ CMs.

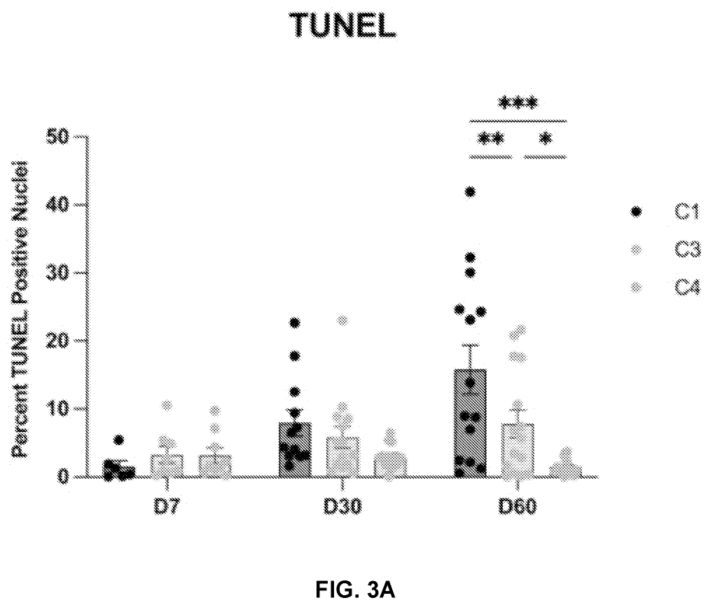

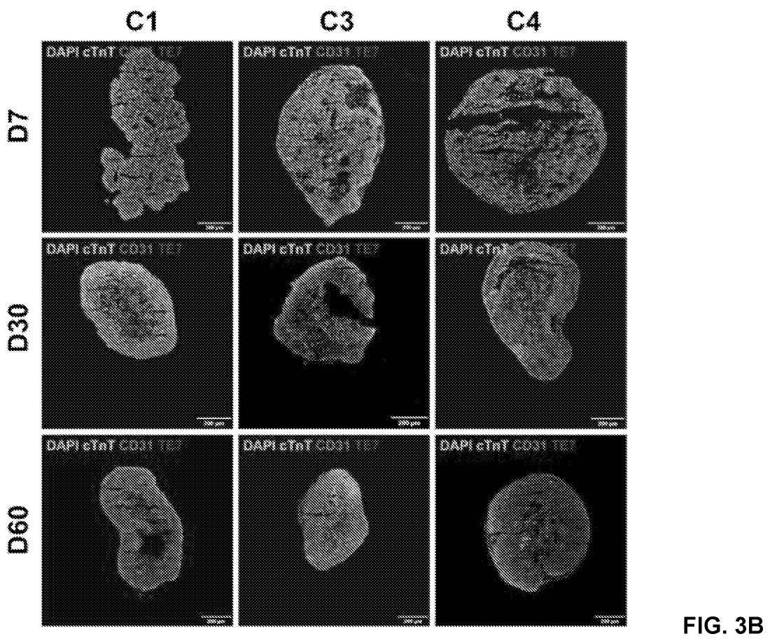

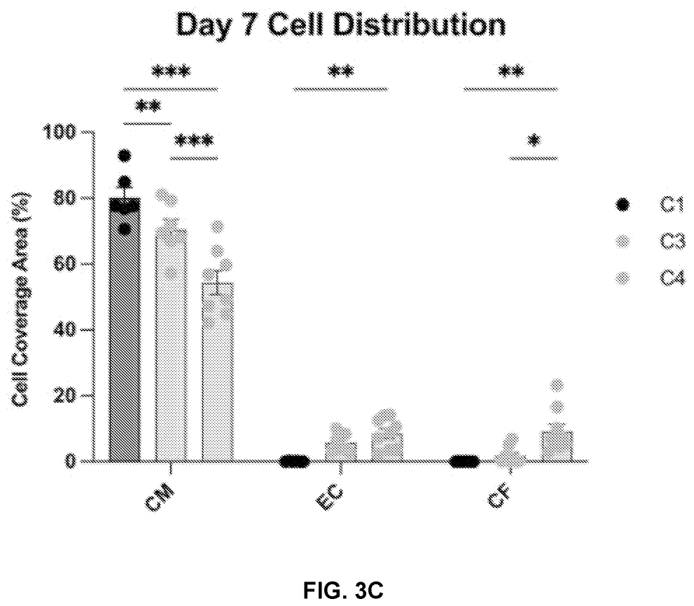

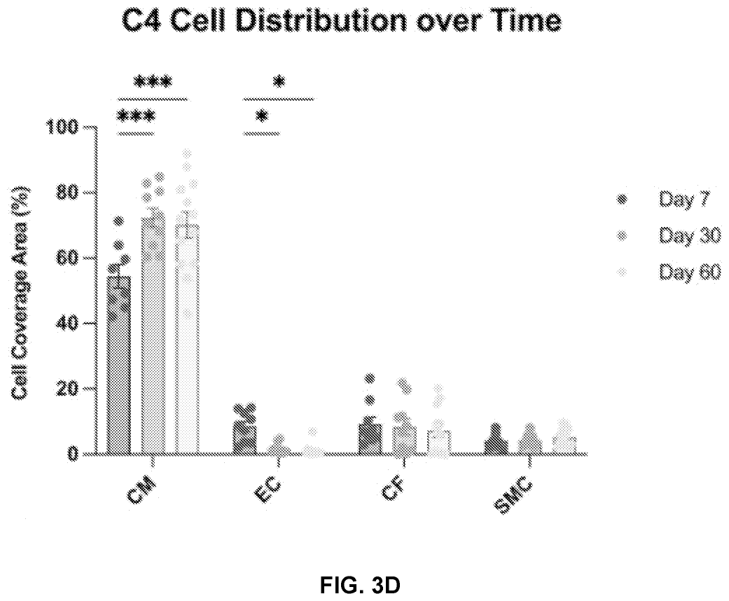

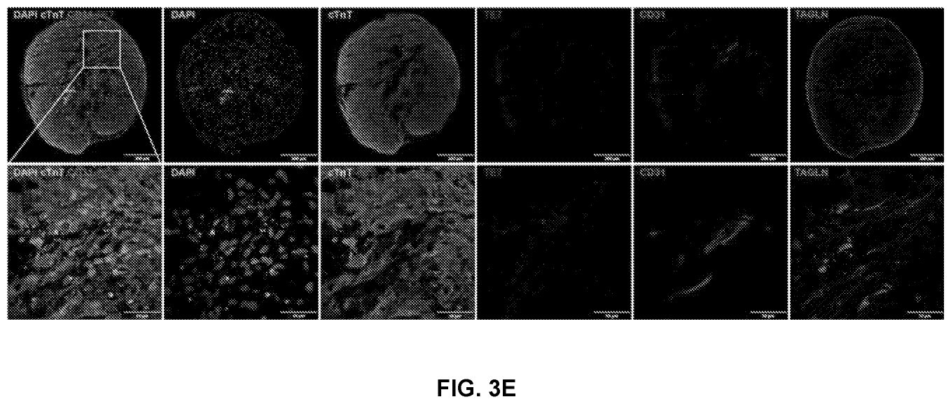

C4 spheroids coalesced into a largely spherical shape within 3-4 days of seeding, while the architecture of the C3 spheroids and (especially) C1 spheroids remained irregular through at least D7 (FIG. 2C). C4 spheroid diameters were also significantly smaller on D7 (C1: 1198±38.2 μm; C3: 1016±21.1 μm; C4: 964±12.6 μm, p<0.001 versus C1 and C3), but the trend was reversed on D30 (C1: 719±11.9 μm; C3: 714±10.4 μm; C4: 804±13.9 μm, p<0.001 versus C1 and C3) and D60 (C1: 676±14.7 μm; C3: 678±8.8 μm; C4: 730±11.7 μm, p<0.01 versus C1, p<0.05 versus C3), when C4 spheroids were significantly larger than the other two constructs (FIG. 2D). These differences may be accounted for by proliferation of non-myocytes in the C3 and C4 groups however, over time all groups showed trends of decreasing diameter and further immunostaining (FIG. 3) did not show significant increases in ECs, SMCs, or CFs. Observations in Masson-trichrome stained sequential sections (FIG. 2E) suggested that the density of muscle cells and fibers increased from D7 to D60 in all constructs, but only C3 and C4 spheroids displayed evidence of collagen formation (FIG. 2F), which is consistent with the absence of ECM-producing ECs, SMCs, or CFs (Lee et al., 2019a; Strikoudis et al., 2019) in C1 spheroids. Furthermore, although assessments of apoptosis (TUNEL staining) did not differ significantly among groups on D7, apoptotic cells became increasingly common in C1 spheroids and (to a lesser extent) C3 spheroids during the culture period, while the proportion of apoptotic cells in C4 spheroids remained stable through at least D60, when apoptotic cells were significantly less common in C4 spheroids than in the other two constructs and in C3 spheroids than in C1 spheroids (FIG. 3A). Apoptotic cells also tended to be located toward the center of C1 spheroids, which suggests that the vascular cells and CFs present in C4 spheroids facilitated access of the culture medium to the spheroid interior. These findings are consistent with previous work supporting the crucial role that non-myocytes play in promoting CM survival particularly where nutrient and oxygen diffusion is limited through release of paracrine factors (Ye et al., 2014; Hodgkinson et al., 2016; Giacomelli et al., 2017; Giacomelli et al., 2020; Munarin et al., 2020; Pretorius et al., 2021).

hiPSC-CMs Occupied a Progressively Larger Proportion 329 of C4 Spheroids Over Time.

The distribution of cell types during the culture period was evaluated in sections stained for the expression of cTnT, CD31, and the fibroblast marker TE-7 (FIG. 3B), as well as with SMC marker transgelin (TAGLIN). On D7, the proportion of cTnT-positive surface area (i.e., CM occupancy) was significantly lower in C4 spheroids than in the other two constructs, and in C3 spheroids than in C1 spheroids (FIG. 3C), which is consistent with the initial composition of cell types used during spheroid construction. In C4 spheroids, CM occupancy increased from D7 to D30, while EC occupancy declined over the same period (FIG. 3D), perhaps because the ECs grew from centrally located clumps on D7 into elongated structures that permeated the entire spheroid on D30 and D60 (FIG. 3E). CM and EC occupancy remained largely stable in C4 spheroids from D30 to D60, while SMC and CF occupancy did not change significantly throughout the culture period. Although some restructuring occurred, immunofluorescent imaging studies illustrated sparse and non-uniform distribution of non-myocytes throughout the time course suggesting that even small amounts of these cells can provide the necessary benefit to CM survival. Low levels of CD31 expression were also observed in C1 spheroids, and both C3 spheroids and C1 spheroids contained a very small number of TE-7− positive cells, perhaps because growth factors present in the culture medium (e.g., VEGF, FGF) induced EC- and CF-like phenotypes in hiPSC-derived cells that were not fully differentiated before the constructs were assembled.

hiPSC-CMs in C4 Spheroids Matured During the Culture Period.

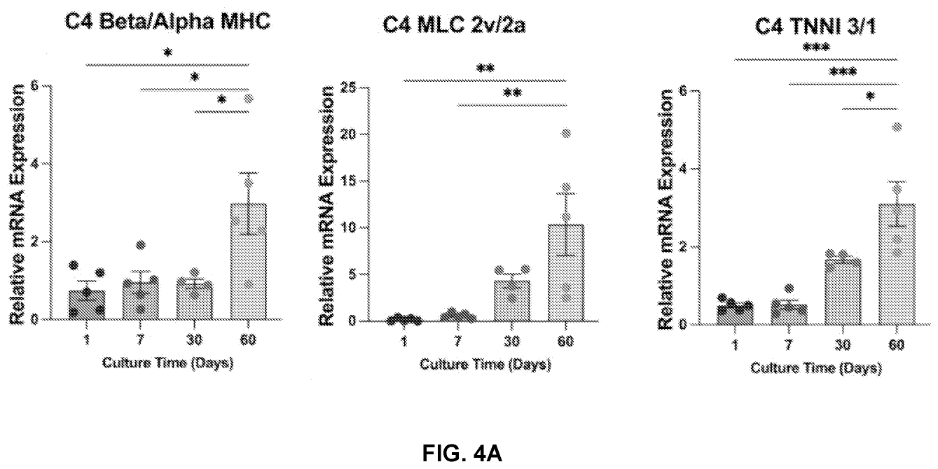

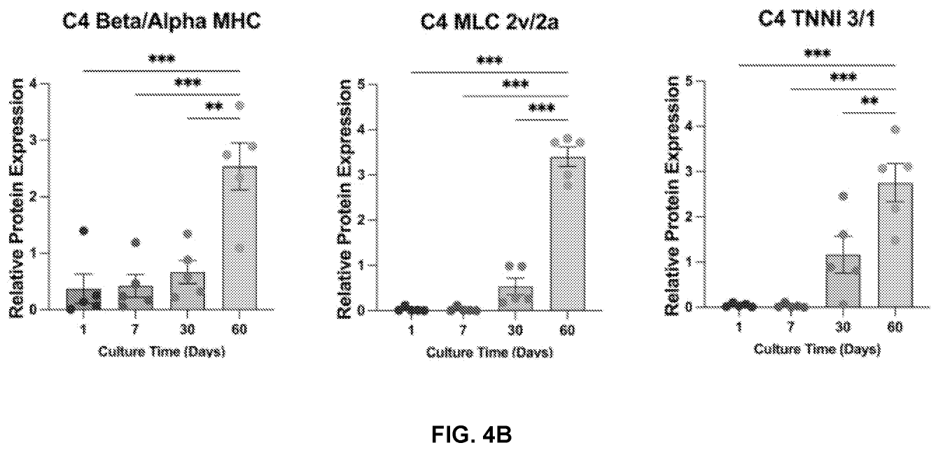

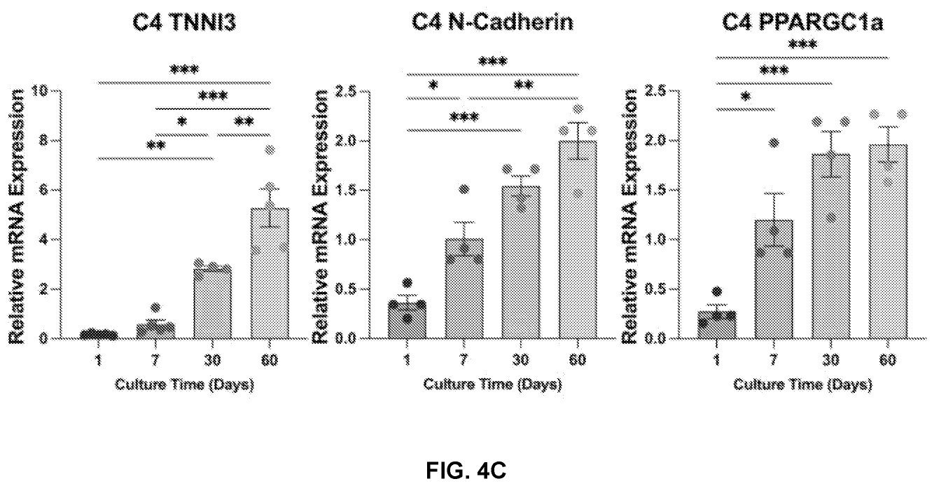

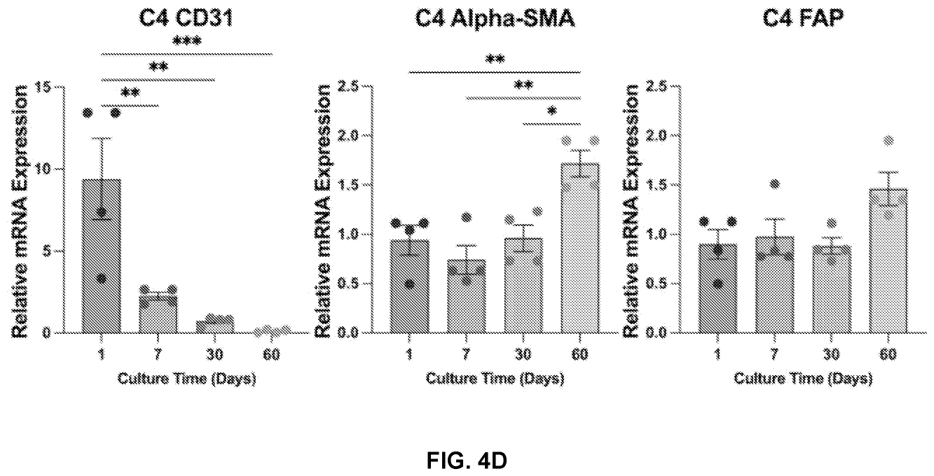

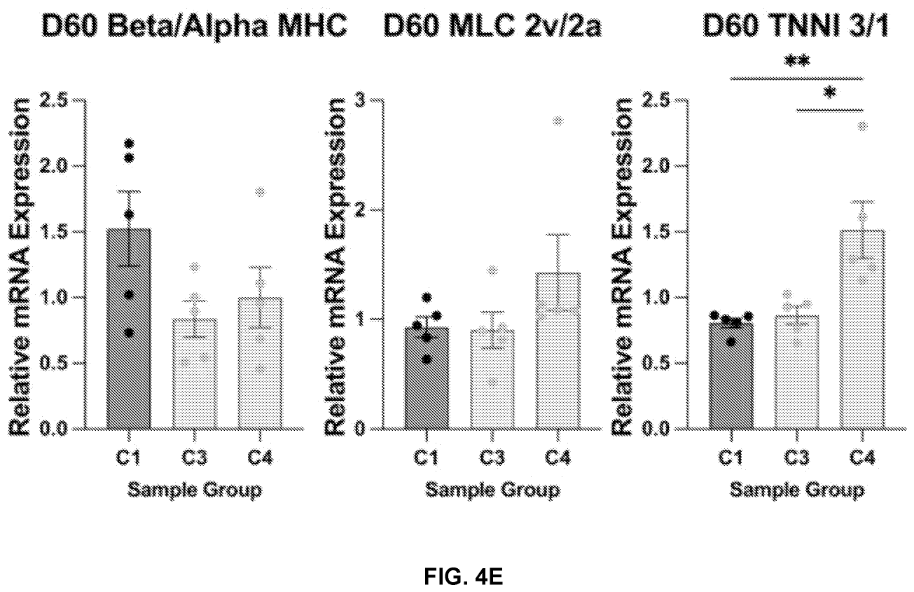

CM maturation and ventricular specification is associated with increases in the ratios of expression for beta versus alpha myosin heavy chain (beta:alpha MHC), ventricular versus atrial myosin light-chain 2 (MLC 2v:2a), and cardiac (type 3) versus slow-skeletal (type 1) troponin I (TNNI 3:1) (Guo and Pu, 2020; Karbassi et al., 2020). When calculated from measurements of either mRNA (FIG. 4A) or protein (FIG. 4B) abundance, each of the three ratios increased significantly in C4 spheroids throughout the culture period, including from D30 to D60 for all except the ratio of MLC 2v:2a mRNA, and the abundance of structural (TNNI3), electromechanical (N-cadherin), and metabolic (peroxisome proliferator-activated receptor gamma coactivator 1-alpha [PPARGC1a]) markers for CM maturation significantly increased through at least D30 (FIG. 4C). Alpha-SMA expression also increased significantly throughout the culture period, while CD31 expression significantly declined, and the expression of fibroblast activating protein (FAP) was largely unchanged (FIG. 4D). Collectively, these observations indicate that the maturation and modification of both CMs and vascular cells continues for at least two months in cultured C4 spheroids.

The Inclusion of hiPSC-ECs, -SMCs, and CFs Increased Sarcomere Maturation and CM Energy Production in Cardiac Spheroids.

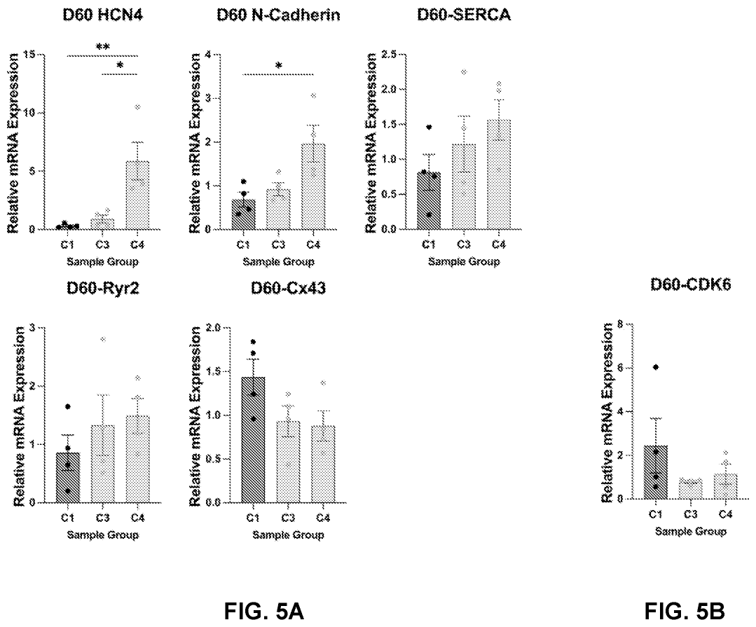

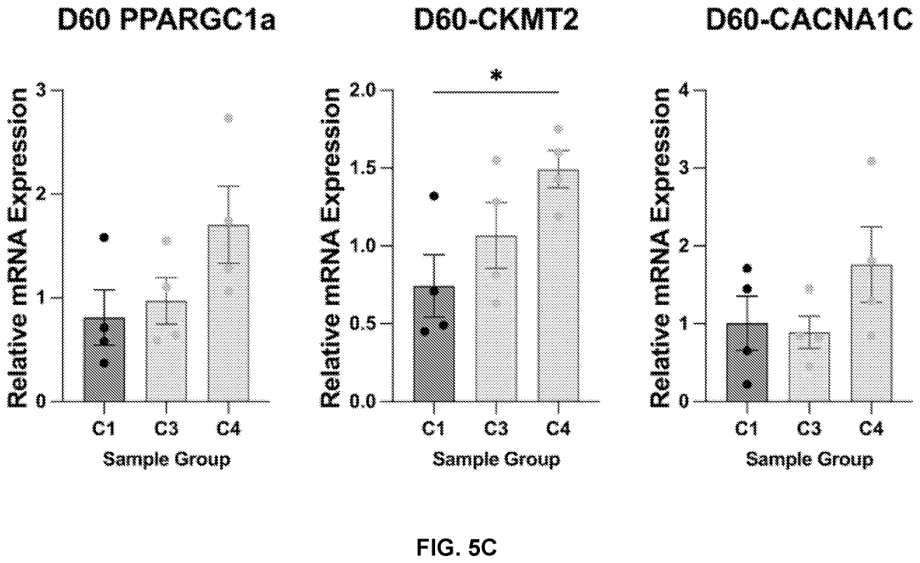

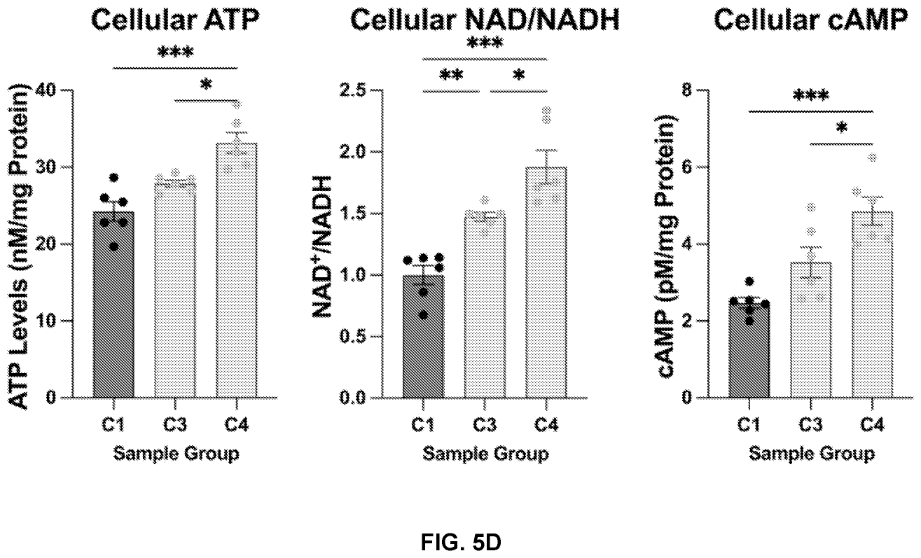

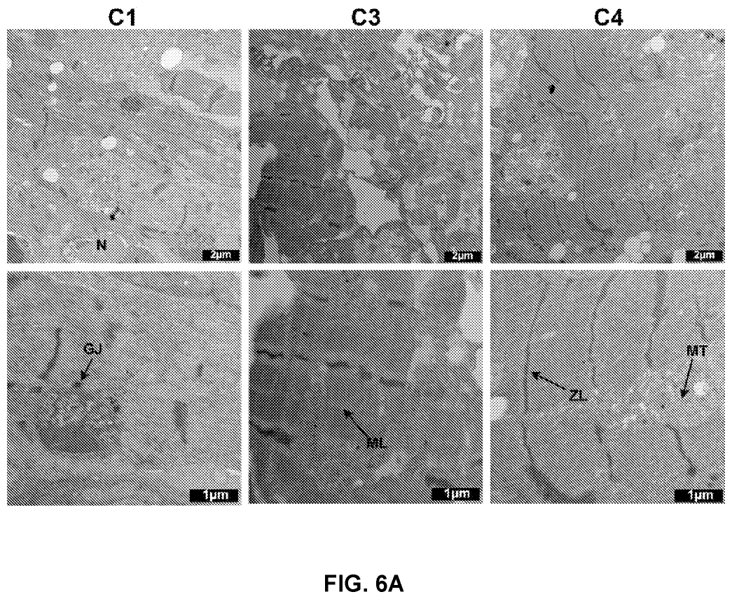

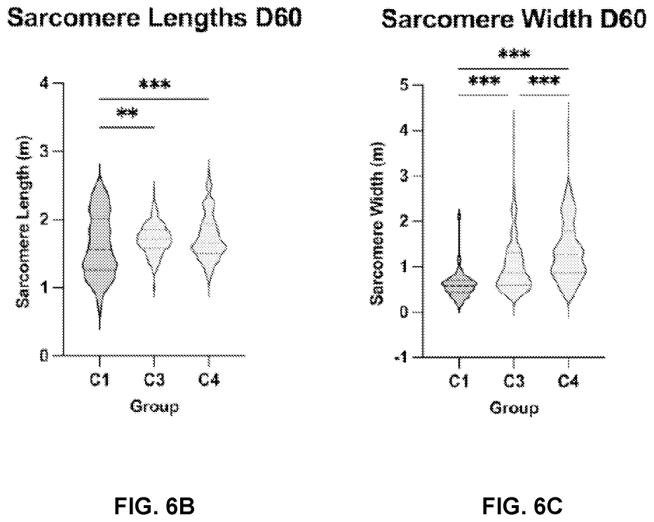

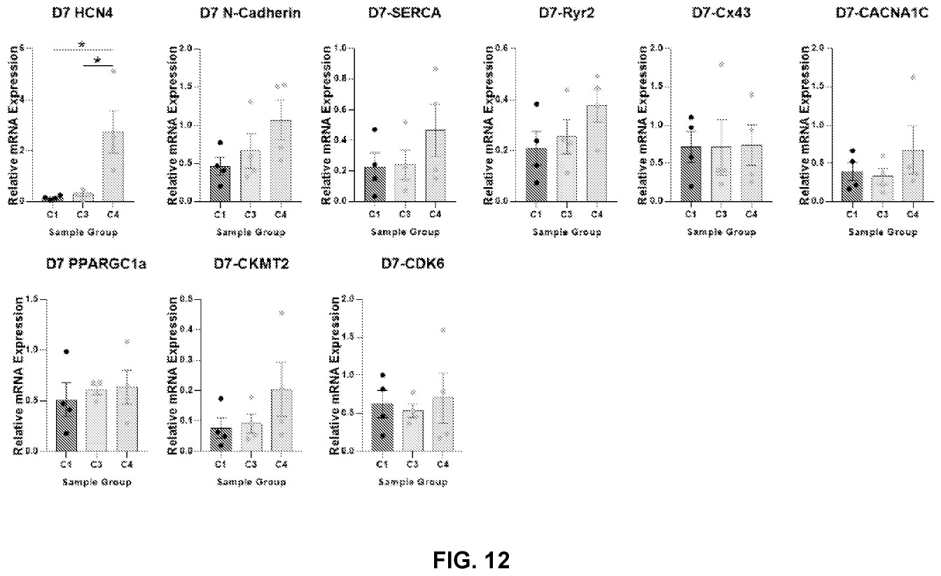

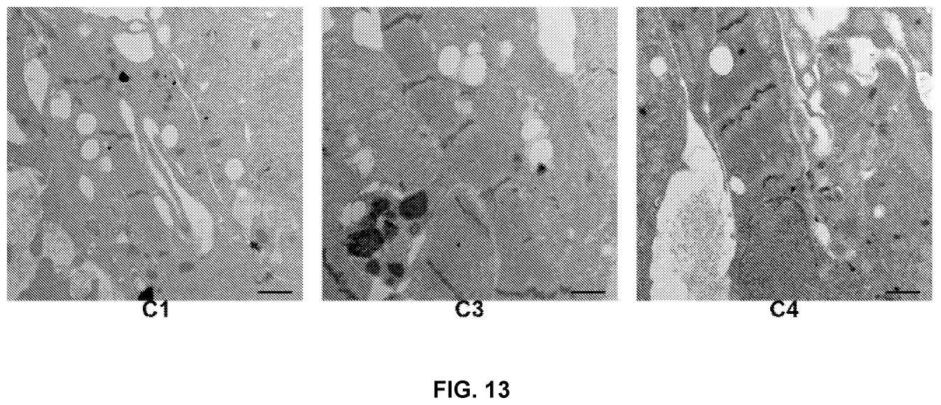

The TNNI 3:1 mRNA ratio was also significantly higher in C4 spheroids than in C3 spheroids or C1 spheroids on D60, but the ratios of beta:alpha MHC and MLC 2v:2a mRNA did not differ significantly among the three groups (FIG. 4E). mRNA levels on D60 for a panel of genes that participate in CM electrical conduction (hyperpolarization activated cyclic nucleotide gated potassium channel 4 [HCN4], N-cadherin, sarcoplasmic/endoplasmic reticulum calcium ATPase [SERCA], ryanodine receptor 2 [Ryr2], connexin 43 [Cx43], calcium voltage-gated channel subunit alpha 1C [CACNA1C]; FIG. 5A) and metabolism (PPARGC1a, creatine kinase, mitochondrial 2 [CKMT2]; FIG. 5B) also tended to be more highly expressed in C4 spheroids; however, the only differences that reached statistical significance were for CKMT2 and N-cadherin, which were significantly greater in C4 spheroids than in C1 spheroids, and for HCN4, which was significantly greater in C4 spheroids than in either of the other two groups. These expression level differences observed on day 60 were found to be mostly absent when the same genes we examined on day 7 after formation (FIG. 12). Notably, mRNA levels for both Cx43 and the cell-cycle regulatory molecule cyclin-dependent kinase 6 (CDK6) (FIG. 5C) appeared to be lower in C4 spheroids than in C1 spheroids, but not significantly, while ATP levels, the ratio of NAD:NADH, and cAMP levels were significantly greater in C4 spheroids than in C1 spheroids (FIG. 5D). Furthermore, observations in transmission electron microscopy (TEM) images collected on D60 confirmed that all three spheroid constructs contained Z-lines and gap junctions, but more complex structures, such as M-lines, I-bands, and A-bands, were observed only in C3 and C4 spheroids. This was notably a qualitative improvement from images collected on spheroids at D30 (FIG. 13). Contractile fibers also appeared to be more organized, and mitochondria better aligned, in C4 spheroids (FIG. 6A), and both sarcomere lengths (FIG. 6B) and widths (FIG. 6C) were significantly greater in C4 spheroids than C1 spheroids. Thus, the inclusion of ECs, SMCs, and CFs in cardiac spheroids tended to promote sarcomere maturation and CM energy production.

Field-Potential Duration and Conduction Velocity were Greater in CMs from C4 Spheroids than from C1 Spheroids.

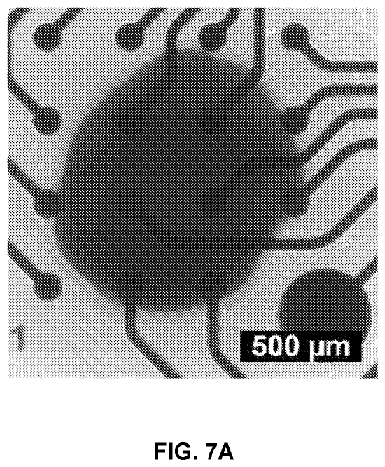

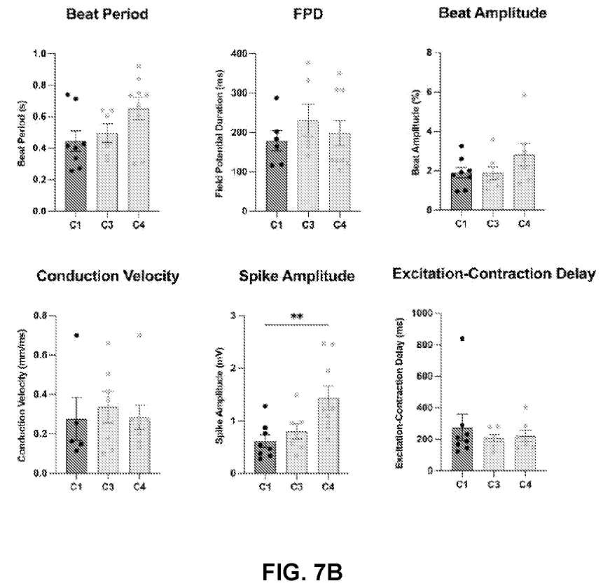

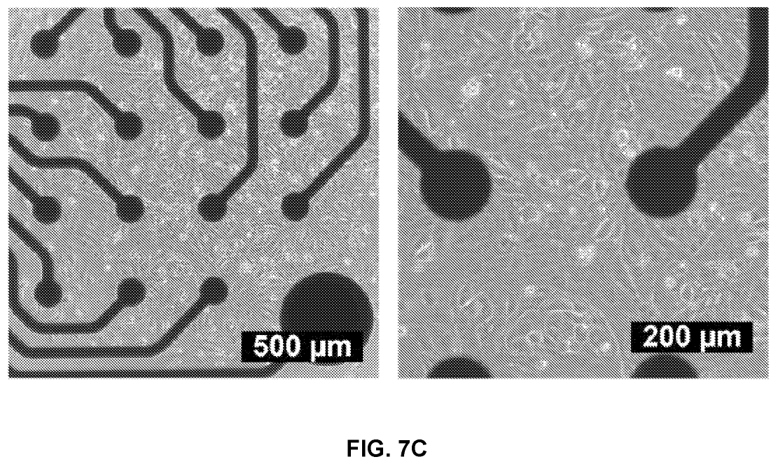

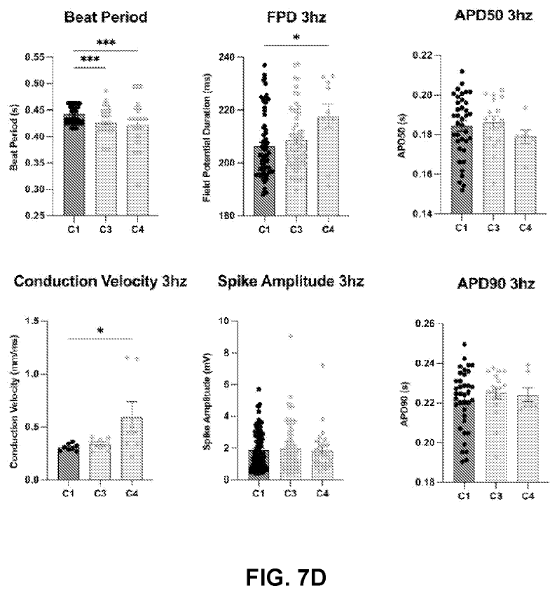

MEA assessments conducted on D30 of whole spheroids (FIG. 7A) indicated that the electromechanical properties of all three constructs were generally similar: spike amplitude measurements were significantly greater in C4 spheroids than in C1 spheroids, but variations among groups in beat period, beat amplitude, field-potential duration, conduction velocity, and excitation contraction delay were small and not significant (FIG. 7B). However, when CMs were isolated from the constructs (FIG. 7C), beat period was significantly shorter for CMs from C3 or C4 spheroids (i.e., C3 or C4 CMs) than from C1 spheroids (C1 CMs), and field-potential durations were significantly longer in C4 than in C1 CMs when the cells were paced at 3 hz (FIG. 7D). Conduction velocities were also significantly greater in C4 than C1 CMs during pacing, likely because the variability among measurements in C4 CMs was exceptionally high, while measurements of spike amplitudes and action-potential durations were similar in CMs from all three constructs.

Discussion

The biological properties and activity of cells in the human heart are reproduced with greater fidelity by cardiac spheroids than by other in-vitro cell-culture systems. Thus, spheroids tend to provide a more accurate platform for modeling cardiac disease or drug development, and the individual cell populations present in cardiac spheroids may be more suitable for tissue engineering and other therapeutic applications. Here, we show that spheroids composed of hiPSC-derived CMs, ECs, SMCs, and CFs can be readily assembled and cultured for up to 60 days in solution; that C4 spheroids were significantly larger than C1 spheroids or C3 spheroids; and that the inclusion of vascular cells and CFs tended to promote sarcomere maturation and CM energy production. Furthermore, whereas apoptotic cells became increasingly common from D7 to D60 in C1 spheroids and C3 spheroids, the proportion in C4 spheroids remained stable throughout the culture period, which suggests that C4 spheroids are sufficiently durable for long-term studies. Notably, all four cardiac-cell types were differentiated from the same line of hiPSCs and, consequently, had the same genetic background, and the ratio of CMs, ECs, SMCs, and CFs (4:2:1:1) in C4 spheroids roughly followed the distribution trends found in human myocardium (Banerjee et al., 2007; Pinto et al., 2016; Gao et al., 2018; Arai et al., 2020; Beauchamp et al., 2020; Daly et al., 2021; Pretorius et al., 2021), but whether this ratio also produces the most native-like cardiac-cell phenotypes in cultured spheroids has yet to be conclusively demonstrated. The present study illustrates that additional optimization of this seeding ratio may be necessary to account for cell loss and restructuring that occurs and techniques for determining the composition in the fully matured tissue may be necessary. Additionally, the ideal ratio for different applications may vary depending on the region or state of myocardium that is being modeled.

The suspension culture system used in this report is broadly compatible with existing research equipment, and quality control checkpoints can be incorporated to maximize reproducibility; thus, our protocol can be readily adapted for a wide range of applications (Mattapally et al., 2018; LaBarge et al., 2019a; LaBarge et al., 2019b; Kim et al., 2020; Daly et al., 2021; Polonchuk et al., 2021) and production facilities (Abbasalizadeh et al., 2017; Adil and Schaffer, 2017; Li et al., 2017; Tomov et al., 2019). Furthermore, techniques for promoting spheroid fusion (Kim et al., 2018; Mattapally et al., 2018) and for the use of spheroids in 3D bioprinting (Duan, 2016; Maiullari et al., 2018; Aguilar et al., 2019; Alonzo et al., 2019; LaBarge et al., 2019b; Qasim et al., 2019; Wang et al., 2021b) have already been established, so the spheroids generated via this protocol could serve as building blocks for even larger and more sophisticated cardiac-tissue constructs, and because ECM production occurred spontaneously in C4 spheroids, exogenously administered ECM components may be unnecessary. However, only ˜50 spheroids per batch were produced in this study, and several of the steps were performed manually, so additional automation will be necessary to maximize productivity on an industrial scale.

The wide application of cardiac spheroid systems has led many groups to develop a range of models for both in vitro and in vivo uses. The microtissues in the present study achieved a greater overall diameter (500-1000 μm) than were previously produced (50-400 μm) with maintained cellular complexity and viability (Beauchamp et al., 2020; Giacomelli et al., 2020; Giacomelli et al., 2021). Although decreased central necrosis was observed in the C4 spheroids, the large diameter and fusion process raises concerns for nutrient and oxygen diffusion (Langan et al., 2016) which will require further assessment in future studies. The use of bioreactor culture environment rather than static culture may provide partial mitigation. Spheroids were also shown to maintain beating and viability over a 60 day period which provides greater longitudinal analysis than the single time points at 28, 20, 15, 30, and 12 days used by others (Ravenscroft et al., 2016; Giacomelli et al., 2017; Israeli et al., 2020; Polonchuk et al., 2021; Thomas et al., 2021). Finally, the protocol of fusing the multiple cell types into larger cardiac spheroids described here uses 96 well plates, a widely used format, and doesn't require CM dissociation, a challenging and labor intensive step, while still maintaining consistent ratios of cell types. This is in comparison to complicated fabrication processes involving complete CM dissociation, hanging drop methods, agarose mold production, and additional centrifugation steps (Nugraha et al., 2019; Buono et al., 2020; Kupfer et al., 2020; Giacomelli et al., 2021; Thomas et al., 2021) and high variability due to spontaneous differentiation compared with the combination of purified cell types (Hoang et al., 2018; Schulze et al., 2019; Israeli et al., 2020) found in other studies.

Although the current study showed improvements in key biomolecular and functional markers a limitation in the data was the lack of functional cardiac metrics at each time point. Particularly, the lack of electromechanical testing at D60 when other biomarkers appeared to show the greatest improvement leaves a gap in maturation characterization. Future iterations of the cardiac spheroid model should consider incorporating data from both early and late time points for all analyses to provide greater longitudinal understanding. Additionally, the media composition chosen was based on previous work with cardiac cell coculture (Giacomelli et al., 2017; Gao et al., 2018; Noor et al., 2019; Pretorius et al., 2021) but was not optimized or fully defined. The use of FBS, although routine, has previously been shown to lead to dedifferentiation of E 464 Cs (Kaupisch et al., 2012; Sriram et al., 2015) and an alternative media formulation may be considered for future model iterations that is cGMP compliant (Zimmermann, 2020).

In conclusion, the experiments in this report demonstrate that C4 spheroids composed of four different hiPSC-derived cell populations (CMs, ECs, SMCs, and CFs) can be efficiently manufactured via a 3D suspension culture that is compatible with a wide range of applications and research equipment. Measures of spheroid size and cell viability were significantly greater in C4 spheroids than in spheroids lacking CFs after 60 days of culture, and the inclusion of vascular cells and CFs tended to promote sarcomere maturation and CM energy production. Future work will continue to investigate methods for improving CM maturity and spheroid yield while incorporating automated processes that are suitable for large-scale production facilities.

TABLE 1
Data collected from MEA analysis of whole and dissociated spheroids

| | | | Spike | | Conduction | Beat | | | |
|---|---|---|---|---|---|---|---|---|---|
| | | Beat | Amplitude | FPD | Velocity | Amplitude | ECD | APD50 | APD90 |
| Format | Group | Period (s) | (mV) | (ms) | (cm/s) | (%) | (ms) | (ms) | (ms) |
| Whole | C1 | 0.45 ± 0.07 | 0.61 ± 0.12 | 179 ± 26 | 27.6 ± 10.9 | 1.90 ± 0.27 | 275 ± 83 | | |
| Spheroid | C3 | 0.50 ± 0.06 | 0.80 ± 0.15 | 231 ± 40 | 33.6 ± 8.0 | 1.88 ± 0.33 | 207 ± 22 | | |
| | C4 | 0.65 ± 0.07 | 1.43 ± 0.23 | 198 ± 32 | 28.5 ± 6.3 | 2.83 ± 0.57 | 222 ± 35 | | |
| Isolated CMs | C1 | 0.44 ± 0.01 | 1.87 ± 0.12 | 206 ± 2 | 30.7 ± 1.1 | 2.02 ± 0.13 | 142 ± 3 | 184 ± 3 | 222 ± 2 |
| (Paced at 3 Hz) | C3 | 0.43 ± 0.01 | 1.95 ± 0.15 | 209 ± 2 | 34.0 ± 1.9 | 2.10 ± 0.19 | 144 ± 3 | 186 ± 3 | 225 ± 3 |
| | C4 | 0.42 ± 0.01 | 1.85 ± 0.24 | 218 ± 5 | 59.3 ± 14.7 | 1.74 ± 0.17 | 146 ± 3 | 179 ± 3 | 224 ± 4 |

TABLE 2
Antibodies used in this study along with their sourcing and dilutions

| | | Catalog | | | |
|---|---|---|---|---|---|
| Antibody Name | Source | Number | RRID | Application* | Dilution |
| Troponin T, Cardiac Isoform Ab-1, Mouse Monoclonal | Thermo Fisher | MS295P | AB_61806 | FC | 1:200 |
| Antibody | | | | | |
| Zenon ™ Mouse IgG1 Labeling Kit | Invitrogen | Z-25002 | AB_2736941 | FC | N/A |
| Alexa Fluor ® 647 Anti-CD31 antibody [JC/70A] | Abcam | ab215912 | AB_2890260 | I | 1:200 |
| Alexa Fluor ® 647 Mouse Anti-Human CD31 | BD Bioscience | 561654 | AB_10896969 | FC | 1:20 |
| PE Mouse Anti-Human CD34 | BD Bioscience | 550761 | AB_393871 | I | 1:5 |
| PE Mouse anti-Human CD144 | BD Bioscience | 560410 | AB_1645502 | I, FC | 1:5 |
| Anti-alpha smooth muscle Actin antibody | Abcam | ab5694 | AB_2223021 | I, FC | 1:200, |
| | | | | | 1:100 |
| Anti-Caveolin-3 antibody | Abcam | ab2912 | AB_2291095 | I | 1:200 |
| Anti-TAGLN/Transgelin antibody | Abcam | ab14106 | AB_443021 | I | 1:200 |
| Anti-Fibroblasts Antibody, clone TE-7 | Sigma | CBL271 | AB_93449 | I, FC | 1:200 |
| Recombinant Anti-Vimentin antibody [EPR3776] | Abcam | ab92547 | AB_10562134 | I | 1:200 |
| Anti-SOX2 antibody | Abcam | ab97959 | AB_2341193 | FC | 1:100 |
| Anti-SSEA4 antibody [MC813-70] | Abcam | ab16287 | AB_778073 | FC | 1:100 |
| Anti-TRA-1-60 (R) antibody [TRA-1-60] | Abcam | ab16288 | AB_778563 | FC | 1:100 |
| Recombinant Anti-Cardiac Troponin T antibody | Abcam | ab91605 | AB_2050427 | I | 1:200 |
| [EPR3695] | | | | | |
| Monoclonal Anti-α-Actinin (Sarcomeric) | Sigma | A7811 | AB_476766 | I | 1:200 |
| Anti-Cardiac Troponin T antibody [1F11] | Abcam | ab10214 | AB_2206574 | I | 1:200 |
| Anti-SERCA2 ATPase antibody | Abcam | ab3625 | AB_303961 | I | 1:200 |
| Anti-Connexin 43/GJA1 antibody - Intercellular | Abcam | ab11370 | AB_297976 | I | 1:200 |
| Junction Marker | | | | | |
| Anti-Troponin I Type 3 Rabbit Polyclonal Antibody | Proteintech | 21652-1-AP | AB_2878898 | WB | 1:600 |
| Anti-Troponin I Type 1 Rabbit Polyclonal Antibody | Proteintech | 16102-1-AP | AB_2206103 | WB | 1:1000 |
| Anti-MYH6 Rabbit Polyclonal Antibody | Proteintech | 22281-1-AP | AB_2736822 | WB | 1:600 |
| MYH7 Rabbit anti-Human, Mouse | Proteintech | 22280-1-AP | AB_2736821 | WB | 1:1000 |
| Myosin Light Chain 2/MLC-2V Polyclonal antibody | Proteintech | 10906-1-AP | AB_2147453 | WB | 1:1000 |
| MYL7 Polyclonal antibody | Proteintech | 17283-1-AP | AB_2250998 | WB | 1:1000 |
| β-Actin Antibody | Cell Signaling | 4967S | AB_330288 | WB | 1:1000 |
| In Situ Cell Death Detection Kit, TMR red | Sigma | 12156792910 | N/A | I | N/A |
| Goat anti-Mouse IgG (H + L) Highly Cross-Adsorbed | Invitrogen | A32723 | AB_2633275 | Secondary | 1:200 |
| Secondary Antibody, Alexa Fluor Plus 488 | | | | Antibody | |
| Goat anti-Rabbit IgG (H + L) Highly Cross-Adsorbed | Invitrogen | A32731 | AB_2633280 | Secondary | 1:200 |
| Secondary Antibody, Alexa Fluor Plus 488 | | | | Antibody | |
| Goat anti-Rabbit IgG (H + L) Highly Cross-Adsorbed | Invitrogen | A32732 | AB_2633281 | Secondary | 1:200 |
| Secondary Antibody, Alexa Fluor Plus 555 | | | | Antibody | |

*FC = Flow Cytometry, I = Immunostaining, WB = Western Blot

TABLE 3
qPCR primers used for quantifying gene expression levels in this study.

| Target | | |
|---|---|---|
| Name | Forward Primer | Reverse Primer |
| SOX2 | GAGGGCTGGACTGCGAACT (SEQ ID NO: 1) | TTTGCACCCCTCCCAATTC (SEQ ID NO: 23) |
| OCT4 | CAGTGCCCGAAACCCACAC (SEQ ID NO: 2) | GGAGACCCAGCAGCCTCAAA (SEQ ID NO: 24) |
| Nanog | TTTGGAAGCTGCTGGGGAAG (SEQ ID NO: 3) | GATGGGAGGAGGGGAGAGGA (SEQ ID NO: 25) |
| Alpha-MHC | CTCCGTGAAGGGATAACCAGG (SEQ ID NO: 4) | TTCACAGTCACCGTCTTCCC (SEQ ID NO: 26) |
| Beta-MHC | ACCAACCTGTCCAAGTTCCG (SEQ ID NO: 5) | TCATTCAAGCCCTTCGTGCC (SEQ ID NO: 27) |
| MLC-2a | GGAGTTCAAAGAAGCCTTCAGC (SEQ ID NO: 6) | AAAGAGCGTGAGGAAGACGG (SEQ ID NO: 28) |
| MLC-2v | ACATCATCACCCACGGAGAAGAGA (SEQ ID NO: 7) | ATTGGAACATGGCCTCTGGATGGA (SEQ ID NO: 29) |
| TNNI 1 | GGTGGATGAGGAGCGATACG (SEQ ID NO: 8) | GCTTCAGGTCCTTAATCTCCCTG (SEQ ID NO: 30) |
| TNNI 3 | GGAGGACACCGAGAAGGAAAAC (SEQ ID NO: 9) | TCAAACTTTTTCTTGCGGCCC (SEQ ID NO: 31) |
| PPARGC1A | GCTTTCTGGGTGGACTCAAGT (SEQ ID NO: 10) | GAGGGCAATCCGTCTTCATCC (SEQ ID NO: 32) |
| CKMT2 | GCTCCGGCTTCAAGACACTC (SEQ ID NO: 11) | TGCGCTTGGAGGAAATAGCC (SEQ ID NO: 33) |
| HCN4 | CCCGGAGGCCGAGGT (SEQ ID NO: 12) | TCAGGTCCCAGTAAAATCTGAAGTC (SEQ ID NO: 34) |
| SERCA | TCACCTGTGAGAATTGACTGG (SEQ ID NO: 13) | AGAAAGAGTGTGCAGCGGAT (SEQ ID NO: 35) |
| RyR2 | TTGGAAGTGGACTCCAAGAAA (SEQ ID NO: 14) | CGAAGACGAGATCCAGTTCC (SEQ ID NO: 36) |
| Cx43 | GGTGACTGGAGCGCCTTAG (SEQ ID NO: 15) | GCGCACATGAGAGATTGGGA (SEQ ID NO: 37) |
| N-Cadherin | AGCCAACCTTAACTGAGGAGT (SEQ ID NO: 16) | GGCAAGTTGATTGGAGGGATG (SEQ ID NO: 38) |
| CACNA1C | TGATTCCAACGCCACCAATTC (SEQ ID NO: 17) | GAGGAGTCCATAGGCGATTACT (SEQ ID NO: 39) |
| CDK6 | GACTGACACTCGCAGCCC (SEQ ID NO: 18) | CAGTCCAGAATCATTGCACCTGAG (SEQ ID NO: 40) |
| CD31 | TCAGACGTGCAGTACACGGA (SEQ ID NO: 19) | GGGAGCCTTCCGTTCTAGAGT (SEQ ID NO: 41) |
| Alpha-SMA | TATCCCCGGGACTAAGACGG (SEQ ID NO: 20) | CACCATCACCCCCTGATGTC (SEQ ID NO: 42) |
| FAP | AGGGATGGTCATTGCCTTGG (SEQ ID NO: 21) | ATCCTCCATAGGACCAGCCC (SEQ ID NO: 43) |
| GAPDH | GTGGACCTGACCTGCCGTCT (SEQ ID NO: 22) | GGAGGAGTGGGTGTCGCTGT (SEQ ID NO: 44) |

TABLE 4
Composition of organoid media and component sourcing for 50 mL total volume.

| Component Name | Volume | Concentration | Sourcing |
|---|---|---|---|
| DMEM/F-12 with GlutaMAX | 47.5 mL | | Thermo Scientific Cat# 10565042 |
| B27 Supplement | 1 mL | 2% | Fisher Scientific Cat# 17-504-044 |
| FBS | 1 mL | 2% | Thermo Fisher Scientific Cat# A4736301 |
| VEGF (50 ng/μL) | 25 μL | 25 ng/mL | Fisher Scientific Cat# PHC9393 |
| FGF (5 ng/μL) | 25 μL | 2.5 ng/mL | R&D Systems Cat# 3718-FB-100 |
| Penicillin-Streptomycin | 500 μL | 1x | Fisher Scientific Cat# 15140122 |

### Example 2: Cyclin D2 Overexpressing and Hypoimmunogenic Cardiomyocyte Spheroid Transplantation Induced Endogenous Myocardial Regeneration in the Pig Hearts after Myocardial Infarction

Methods

Generation of HLA I/II Knockdown and CCND2 Over-Expressing hiPSCs (HLA-KO/CCND2-OEhiPSCs)

hiPSC-05 established from female human cardiac fibroblasts (Zhang L, et al. Circ Heart Fail. 2015 8(1):156-66) was engineered to produce FILA-K° hiPSCs using CRISPR/Cas9 system as described (Mattapally S, et al. J Am Heart Assoc. 2018 7(23):e010239). To overexpress CCND2 in FILA-K° hiPSCs, a CCND2 gene fragment was digested from a lenti-α-MHC/CCND2 plasmid which was constructed as described previously (Zhu W, et al. Circ Res. 2018 122(1):88-96; Zhao M, et al. Circulation. 2021 144(3):210-28). Briefly, a full-length of CCND2 gene extracted from 1% agarose gel was subcloned into a new lenti-plasmid carrying α-MHC promotor (Addgene #21231, USA) using Age I and Asc I restriction endonucleases. This generated a new plasmid that had CCND2 gene expression under MHC promotor (pMHC/CCND2). Then, HEK 293T cells were transfected with pMHC/CCND2 and two lentiviral packaging plasmids, sPAX2 (Addgene, 11260) and MD2.G (Addgene, 12259) plasmids using Lipofectamine™ 3000 Transfection Reagent (Thermo Fisher, USA) to generate the lentiviral vectors carrying MHC/CCND2 (lenti-CCND2) (Shadrin I Y, et al. Nat Commun. 2017 8(1):1825).

HEK293T supernatants were harvested at 48 hours and 72 hours after transfection. Purified lentiviruses were obtained by mixing with 4× Lentivirus Concentration Solution and aliquot after centrifuge as directed by the protocol of M DAnderson Center.

^{HLA-KO}hiPSC cells were cultured in mTeSR-Plus medium supplemented with lentivirus containing CCND2 gene for 48 h followed by 5 μg/mL Blasticidin for another 48 h. Then cells were washed with DPBS and cultured in fresh mTeSR-Plus supplemented with 5 μg/mL Blasticidin for four days with daily medium change. After Blasticidin treatment, each single colony was manually picked and expanded in culture as described previously (Zhu W, et al. J Vis Exp. 2017 (120); Gao L, et al. Circ Res. 2017 120(8):1318-25).

^{HLA-KO/CCND2-OE}hiPSCs Characterization

To determine pluripotency of ^{HLA-KO/CCND2-OE}hiPSCs, fluorescence immunostaining and teratoma formation assay were performed. Karyotyping was employed to determine whether ^{HLA-KO/CCND2-OE}hiPSCs had any abnormal chromosomes after engineering.

Immunofluorescence

Fluorescence immunostaining of ^{HLA-KO/CCND2-OE}hiPSCs were performed to determine pluripotent stem protein expressions as described (Zhang L, et al. Circ Heart Fail. 2015 8(1):156-66). Briefly, cells were fixed with 4% paraformaldehyde for 20 minutes at room temperature, permeabilized in 0.1% Triton X-100 at −20. ° C. for 10 minutes, and then blocked with UltraV block (Thermo Scientific) for 7 minutes. After that, ^{HLA-KO/CCND2-OE}hiPSCs were incubated with rabbit anti-OCT4 (ab19857, Abcam, USA), or rabbit anti-SOX2(ab97959, Abcam, USA), or mouse anti SSEA4 (ab16287, Abcam, USA) primary antibodies in 10% donkey serum (DS) overnight at 4° C. On the second day, cells were incubated with corresponding secondary antibodies conjugated with FITC or TRITC for 1 hour at room temperature. At last, cells were stained with 4′,6-Diamidino-2-Phenylindole (DAPI), washed, and mounted. Images were taken using Olympus IX83 microscope.

Teratoma Formation Assay

Teratoma formation assay was assessed as described previously (Hentze H, et al. Stem Cell Res. 2009 2(3):198-210; Gropp M, et al. PLoS One. 2012 7(9):e45532). Briefly, 2 million ^{HLA-KO/CCND2-OE}hiPSCs were intramuscularly implanted into the hind leg of SCID mice. Two months later, teratoma was harvested, embedded into paraffin, sectioned, and stained with hematoxylin and eosin to visualize cell populations differentiated from three germ layers.

Karyotyping

Karyotyping was assessed by WiCell Research Institute. Briefly, ^{HLA-KO/CCND2-OE}hiPSCs were seeded into T25 flasks, and karyotyping was performed when cells reached 40 to 60% confluence. 20 Metaphase cells were analyzed.

HiPSC-CM Differentiation and Purification

Both ^{HLA-KO}hiPSCs and ^{HLA-KO/CCND2-OE}hiPSCs were differentiated into hiPSC-CMs in monolayer or in 3D Spheroids culture as described previously (Zhao M, et al. Circulation. 2021 144(3):210-28; Kahn-Krell A, et al. Front Bioeng Biotechnol. 2021 9:674260; Kahn-Krell A, et al. Front Bioeng Biotechnol. 2022 10:908848). For monolayer differentiation, the differentiation protocol of hiPSCs into CMs had been described (Lian X, et al. Proc Natl Acad Sci USA. 2012 109(27):E1848-57). Briefly, the differentiation of ^{HLA-KO}hiPSC and ^{HLA-KO/CCND2-OE}hiPSC was induced by 10 μM CHIR99021 on day 0. Twenty-four hours later, the differentiation medium was replaced with 1 μM CHIR99021 for 48 hours. Then, the medium was replaced with 10 μM IWR and cultured for 48 hours. On day 5, the cells were cultured in RPMI supplemented with B27 without insulin (B27−) for 48 hours. On day 7, the medium was changed to RPMI/B27. Generally, spontaneously beating CMs was observed on days 7-8 after differentiation.

3D spheroid differentiation was performed as described (Kahn-Krell A, et al. Front Bioeng Biotechnol. 2022 10:908848). Briefly, ^{HLA-KO/CCND2-OE}hiPSC were seeded into Erlenmeyer flask on a Belly Dancer orbital shaker (IBI Scientific, USA) and cultured in 3D TeSR E8 seed media (STEMCELL Technologies, USA). On differentiation day 0, cells were cultured in RPMI/B27− supplemented with 6 μM CHIR99021. On day 1, 80% of the media was replaced with fresh RPMI/B27− for 48 hours. On day 3, 70% of the differentiation medium was replaced with fresh RPM I/B27− supplemented with 5 μM IWR. On day 5, the differentiation medium was replaced with fresh RPMI/B27 ⁻. On day 7, medium will be replaced with RPMI/B27. Generally, CM spheroids started beating around day 7 after differentiation.

To purify hiPSC-CMs, metabolic selection using lactic acid were performed on differentiation day 10 as described (Tao Z, et al. Cardiovasc Res. 2021 117(6):1578-91; Tohyama S, et al. Cell Stem Cell. 2013 12(1):127-37). Briefly, hiPSC-CMs were rinsed with DPBS and cultured in RPMI 1640 without glucose supplemented with 4 mM lactate (Sigma, 252476), 1×GlutaMAX (Gibco, 35050061) and 1×MEM Non-Essential Amino Adds Solution (Thermo Fisher Scientific, 11140050) for 6 days. After lactate acid treatment, cells were recovered using 10% RPMI/B27⁺ for 24 hours. Then purified cells were used for experiments.

Flow Cytometry

Flow cytometry was employed to determine the purity and pluripotent markers in CM spheroids. CM spheroids were dissociated using STEMdiff™ Cardiomyocyte Dissociation Medium (Stemcell Technologies, 05026). Cells were resuspended in Fixation/Permeabilization Solution (BD, 554714) at 4° C. for 20 minutes, washed twice in 1×BD Perm/Wash buffer, and blocked using 2.5 μg Human BD Fc Block (BD, 564219) for 10 minutes at room temperature. Then cells were stained with respective primary antibodies and secondary antibodies. At last, cells were resuspended in 2% fetal bovine serum (FBS)/DPBS solution and analyzed by BD LSRFortessa. The cells with adequate size and granularity were included for analysis (Ye L, et al. Exp Biol Med. 2007 232(11):1477-87; Ye L, et al. Circulation. 2007 116(11 Suppl):1113-20). The total events for each flow analysis was 10,000.

Quantitative RT-PCR

Total RNA was extracted from ^{HLA-KO}hiPSC-CMs and ^{HLA-KO/CCND2-OE}hiPSC-CMs using RNeasy Mini Kit (Qiagen, 74104) according to the manufacturer's instructions. cDNA was synthesized using ProtoScript II Reverse Transcriptase (NEB, M0368L) according to the manufacturer's protocol. Quantitative PCR was performed using PowerUp SYBR Green Master Mix (Applied Biosystems, A25776) and analyzed using a QuantStudio Real-Time PCR cycler (Applied Biosystems). Gene expression level was quantified by the ΔΔCt method using GAPDH as the housekeeping gene.

Western Blotting

Protein was extracted from CMs by adding M-PER™ Mammalian Protein Extraction Reagent (Thermo Fisher Scientific, 78501) supplemented with Halt™ Proteinase and Phosphatase Inhibitor Cocktail (Thermo Fisher Scientific, 78442). To extract protein from tissues, 50 mg heart tissue harvested from the border zone of infarct were cut into small pieces and minced in liquid nitrogen using pestle/mortar. Minced samples were lysed in PhoshoSafe Extraction Ragent (Novagen, 71296) and homogenized. Then, the lysates were vortexed every five minutes for 30 minutes and centrifuged at 15,000 g for 15 min at 4° C. to collect clear supernatant.

Protein concentration was determined using a Pierce BCA Protein Assay Kit (Thermo Fisher Scientific, 23225). Western Blot was performed as described (Ye L, et al. Diabetologia. 2009 52(9):1925-34; Su L, et al. Stem Cell Research & Therapy. 2022 13(1):13). Two μg protein of each sample were loaded each lane on a 4-20% Mini-Protean TGX Stain-Free gel (Bio-Rad, USA) and transferred to a 0.2 μm PVDF membrane using the Trans-Blot Turbo RTA Mini LF PVDF Transfer Kit (Bio-Rad, 1704274). The membrane was blocked 5% of non-fat dry milk (Bio-Rad) in TBST (0.1% Tween-20) for 30 minutes at room temperature and incubated with primary antibody overnight at 4° C. After washing membrane was incubated with HRP-conjugated secondary antibody (1: 4000) for 30 minutes at room temperature. The binding of the specific antibody was detected using the ECL Chemiluminescent Reagent (GE Healthcare Amersham) and visualized using ChemiDoc™ MP Imaging System (Bio-Rad, USA). The protein expression level was normalized by GAPDH or β-tubulin and expressed as percentage of GAPDH or β-tubulin.

Immunostaining of CM Spheroids

CM spheroids were fixed in 4% formaldehyde for 30 min at room temperature, immersed in 30% sucrose at 4° C. overnight. Then, spheroids were embedded in Tissue-Plus O.C.T Compound (Fisher Scientific) for sectioned. Cryo-sections of 10 μm thickness were used for immunostaining. After permeabilization and blocking, samples were stained with rabbit anti-sarcomeric alpha actinin (α-SA) (ab137346, Abcam) and mouse anti-human cardiac troponin T (cTnT, MAB1874, R&D Systems) overnight at 4° C. On the second day, sections were incubated with donkey anti-mouse IgG conjugated with fluorescein (FITC) or anti-rabbit IgG conjugated with Rhodamine secondary antibodies diluted in 10% DS at room temperature for 1 h. at last, sections were washed, mounted in VectaShield Vibrance Antifade Mounting Medium with DAPI, and visualized under Confocal Microscope (Confocal FV3000, Olympus).

MHCK7-GCaMP6 Lentivirus Production and Transduction

To determine the electrocoupling of transplanted ^{HLA-KO/CCND2-OE}hiPSC-CM spheroids with pig host CMs, ^{HLA-KO/CCND2-OE}hiPSC-CM spheroids were transduced Lentivirus carrying MHCK7-GCaMP6-GFP. A lentiviral plasmid, pRRL-MHCK7-GCaMP6 (#65042, Addgene) was bought from Addgene (Madden L, et al. Elife. 2015 4:e04885). HEK 293T cells were transfected with pRRL-M HCK7-GCaMP6 and two lentiviral packaging plasmids, sPAX2 and MD2.G plasmids, to generate the lentiviral vectors carrying GCaMP6-GFP (Shadrin I Y, et al. Nat Commun. 2017 8(1):1825). Purified and concentrated MHCK7-GCaMP6-GFP virus were used to transduce ^{HLA-KO/CCND2-OE}hiPSC-CM spheroids for 24 hours.

Preparation of Spheroids Conditioned Media for Angiogenesis Array Assessment

To determine profiles of paracrine factors released by CM spheroids, hiPSC5-CM spheroids and ^{HLA-KO/CCND2-OE}hiPSC-CM spheroids (4×10⁶ cells) were washed thrice with DPBS and cultured in 3 mL RPMI for 48 hours. Then, the conditioned media was collected, centrifuged, filtered using 0.22 μm syringe, and stored at −80° C. Protein released into conditioned media were identified using a Human Angiogenesis Antibody Array Q 1000 (RayBiotech) described as previously (Lou X, et al. Cardiovasc Res. 2023 119(4):1062-76).

Follistatin Treatment

^{HLA-KO}hiPSC-CMs (i.e. 1×10⁴) on day 40 after differentiation were seeded into individual well of 8-well chamber slides and cultured in RPMI/B27 medium supplemented with 0, 100, 200, and 250 ng/mL follistatin for 48 hours. Then cells were fixed in 4% formaldehyde, permeabilized with 0.1% Triton 100×, and stained with mouse anti-aurora B kinase and rabbit anti-cTnT antibodies. The number of ^{HLA-KO}hiPSC-CMs underwent cytokinesis was quantified as the number of cTnT⁺ CM expressing ABK with disassembled sarcomere and cleavage furrow per mm 2 (Ye L, et al. Circulation. 2018 138(24):2798-808).

To determine whether follistatin can modulate YAP protein expression and activity, hiPSC-CM were collected at 0, 15, 30, 60, and 180 min after cultured with 200 ng/mL follistatin. Western Blot was performed to determine pYAP, YAP, and GAPDH protein expression level.

Western Blot

Purified hiPSC5-CMs (2×10⁵) seeded on 12-well plate were treated 200 ng/ml Follistatin or without Follistatin for different timepoint (0 h, 15 min, 30 min, 60 min, 180 min and 360 min). Cells lysis were performed using PhosphoSafe™ Extraction Reagent as the manufacture's protocol. After the protein was transferred the membrane and blocked with 5% skim milk, the membranes were incubated with primary antibody: Phospho YAP (1:1000), GAPDH and YAP after stripped for overnight incubation. Imaging were performed after the corresponding secondary antibodies incubation.

hiPSC-CM Proliferation In Vitro

Purified hiPSC5-CMs (1×10⁵) grown on 12-well plate (more than one month after differentiation was initially) were treated with RPMI/1640 supplemented with different concentration of follistatin (0 ng/mL, 100 ng/mL and 200 ng/mL for 16 days with media change every 2 days. Cell number was quantified by an automatic cell counter (Invitrogen, Countess 3)

Porcine Heart Model of Ischemia/Reperfusion Injury and Spheroids Transplantation

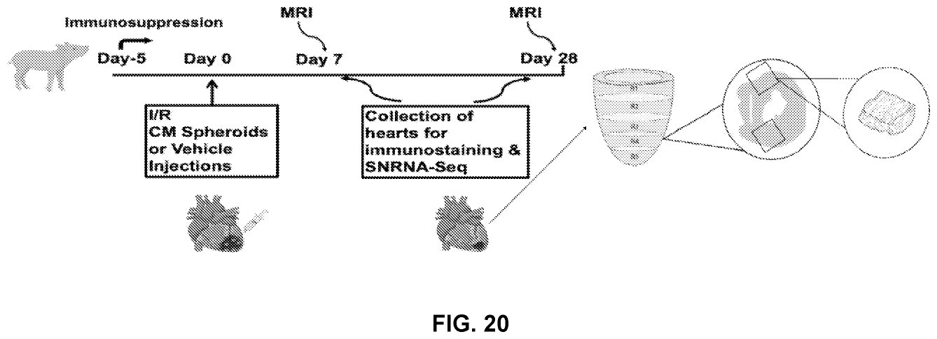

To determine therapeutic efficacy of ^{HLA-KO/CCND2-OE}hiPSC-CMs for the treatment of cardiac injury, a porcine heart model of ischemia reperfusion (I/R) was developed (Ye L, et al. Cell Stem Cell. 2014 15(6):750-61; Xiong Q, et al. Circulation. 2013 127(9):997-1008; Xiong Q, et al. Circ Res. 2012 111(4):455-68). A schematic presentation of pig study was shown in FIG. 20. All animal procedures and protocols were approved by the Institutional Animal Care and Use Committee (IACUC), University of Alabama at Birmingham and performed in accordance with the Guide for the Care and Use of Laboratory Animals from National Institutes of Health (NIH publication No 85-23). Female or male Yorkshire pigs with 10-20 kg body weight received I/R injury and were randomly assigned to receive either basal medium injection (I/R Group) or ^{HLA-KO/CCND2-OE} hiPSC-CM spheroid injection (Spheroid Group). Sham-operated animals will undergo all surgical procedures for I/R surgery except that the coronary artery was not ligated.

One mL RPM11649 medium alone or containing 25×10⁶ CMs of ^{HLA-KO/CCND2-OE} hiPSC-CM spheroids was intramyocardially injected into the ischemic LV wall. Animals in each group had cardiac magnetic resonance imaging (cMRI) to determine infarct size and heart function at weeks 1 and 4 after treatment. Animals were euthanized either on weeks 1 and 4 to have their hearts harvested for electrocoupling mapping, scRNAseq, Western Blot, and immunohistology studies.

To improve engraft, immunosuppression drugs were used. Cyclosporine A was administered orally twice per day (i.e. 15 mg/kg in AM and 30 mg/kg in PM) starting 5 days before cell transplantation until 4 weeks. Abatacept (i.e. 12.5 mg/kg; Bristol-Myers Squibb) was given on the day of I/R surgery and once every 2 weeks thereafter. Methylprednisolone (i.e. 250 mg, Pfizer, Mississauga) was given on the day of I/R surgery and once daily (i.e. 125 mg) thereafter.

Cardiac Magnetic Resonance Imaging

Pigs had cMRI in the Research MRI Core of UAB at 1 and 4 weeks after surgery and treatment [15, 18, 34-36]. Animals were anesthetized and placed in a supine position within a 3.0 T whole body MRI scanner (GE signa horizon software 9.1). A phased-array 4-channel surface coil was applied above the heart. The short- and long-axes of the heart were identified. Global function was computed from the short-axis cine images by semi-automated segmentation of the LV endocardial and epicardial borders (from base to apex) at both end-diastole and end-systole using CAAS MRV 3.4 analysis software (Pie Medical Imaging, Netherlands). Gadolinium-based contrast agent Magnevist (0.3 mmol/Kg, gadopentetate dimeglumine, Bayer Healthcare, Bayer) was injected intravenously. After 10 min, late gadolinium enhancement inversion-recovery gradient echo sequences were acquired at the same positions as the cine images. Infarct size was calculated from the late gadolinium enhancement images with scar surface area expressed as a percentage of the total LV surface area (Ye L, et al. Cell Stem Cell. 2014 15(6):750-61; Ye L, et al. Circulation. 2018 138(24):2798-808).

Tissue Harvest and Engraftment of hiPSC-CMs

Animals were euthanized either at week 1 or 4 after I/R and treatment. Pig hearts were arrested by injecting 100 mg/Kg KCl under anesthesia and explanted. The LV was cut into 5 or 6 short-axis rings (R1 to R6 is basal to apex). The CM spheroid injected areas of the R3, R4, and R5 were collected as infarct zone and infarct border zone (BZ), while lateral wall of the R1 and R2 were collected as remote zone (RZ). Heart tissue blocks were embedded in OCT and used for cryo-section. Tissues were cut into 8 μm thickness sections, stained with multiple antibodies to identify human CMs in porcine heart. After overnight incubation with primary antibody at 4° C., samples were washed and incubated with corresponding secondary antibodies conjugated with fluorescence for 1 hour at room temperature. Then, tissues were washed and mounted in VectaShield Vibrance Antifade Mounting Medium with DAPI and examined using confocal microscopy (Confocal FV3000, Olympus). Every fifteenth section slides from each tissue block were used for immunostaining. At least thirty-five tissue section slides of each tissue block were evaluated.

Optical Mapping of CM Spheroid in the Pig Hearts

Following sternotomy, the heart was arrested by injecting ice-cold cardioplegia (110 mL NaCl, 1.2 mL CaCl₂, 16 mM KCl, 16 mM MgCl₂, 10 mM NaHCO₃, pH=7.4) through aortic cannulation. The heart was covered with ice-cold 0.9% saline slushes before explant.

The heart was explanted and maintained in ice-cold 0.9% saline solution for 5 min. The atria and right ventricle were removed and the remaining left ventricle was cut into 5 rings. The 1.5 cm×1.5 cm tissues were obtained from the spheroid injected area near the border zone. The tissues were cut into 600 μm thick slices using a high precision vibratome (7000smz-2, Campden Instruments Ltd. UK). The slices were then maintained in a mapping chamber circulated with oxygenated Tyrode solution at 37° C. The slices containing injected spheroids were optically mapped under blue LEDs excitation and calcium fluorescence for imaging the GCaMP6 transfected CM spheroids.

Porcine Cardiomyocyte Proliferation

To determine endogenous pig CM proliferation, pig heart tissue cryo-sections were immunostained with Ki67, pH3, aurora-B kinase, and YAP as described. CM cytokinesis was determined by dual immunostaining for mouse anti-ABK and goat anti-cTnI. CM mitosis was determined by dual immunostaining for Ki67 or pH3 with cTnI. CM cytokinesis was quantified as the number of cTnI⁺ CM expressing ABK with disassembled sarcomere and cleavage furrow per mm 2 (Ye L, et al. Circulation. 2018 138(24):2798-808). CM mitosis was quantified as the number of cTnI⁺ CM expressing Ki67 or pH3 per mm 2. YAP expression in CM was evaluated as the percentage of YAP+ CM nuclei. Similarly, every fifteenth section slides from each tissue block were used for immunostaining. At least thirty-five tissue section slides of each tissue block were evaluated.

Single Nuclei RNA-Sequencing and Data Analysis

Nucleus were isolated from heart tissues as previously described [37, 38]. Briefly, fresh or frozen tissues (100-200 mg) were cut into small pieces in cold UW solution or lysis buffer (10 mM Tris-HCl 8.0, 5 mM CaCl₂, 3 mM Mg(CH3COO)₂, 0.5 mM EGTA, 2 mM EDTA, 1 mM PMSF, 1 mM DTT and 50 U/mL RNase inhibitor in 0.32 M sucrose buffer). Then the tissue samples were homogenized for 18 seconds in 10 mL of lysis buffer and placed on ice for 10 min. After filtered with 100 μm and 70 μm strainers, the tissue lysate was centrifuged at 700 g for 10 min at 4° C.; and resuspended in 5 ml nuclei wash and resuspension buffer (1.0% bovine serum albumin [BSA] supplemented with 80 U/mL RNase inhibitor). The resuspension was filtered with 40 μm strainer, and the nuclei were harvested by centrifuging at 700 g for 10 min at 4° C. The nuclei pellet was resuspended in 400 μl of 1.0% BSA containing 200 U/mL RNase inhibitor. Then nuclei were stained with propidium iodide for 5 min, selected using flow cytometry, and processed via the 10× Genomics Single-Cell Protocol (Nakada Y, et al. Circulation. 2022 145(23):1744-7; Nguyen T, et al. Front Bioeng Biotechnol. 2022 10:914450).

Considering that the tissue might contain transplanted ^{HLA-KO/CCND2}hiPSC-CM cells before collecting single-nuclei RNA sequencing (snRNAseq) data; we applied a method to separate host (pig) and graft (^{HLA-KO/CCND2}hiPSC-CM) cells in snRNAseq data [39]. Briefly, we used the Cell Ranger v.6 toolkit to build a combined reference genome between the Human GRch38 and Pig Sscrofa11.1 genomes. In each cell, all RNA transcripts were mapped to this combined genome; the barcodes (a cell identifier) having less than 500 transcripts were removed; and the number of map-to-human and map-to-pig transcripts were counted. Cells having the ratio between map-to-pig-genome transcripts and map-to-human-genome of 5 and above were the host pig cells; otherwise, the cells were ^{HLA-KO/CCND2}hiPSC-CM. Although ^{HLA-KO/CCND2}hiPSC-CM were detected, their number was very small (<0.1%) compared to the number of pig cells. Therefore, the later analyses only focus on pig cells.

The raw-read (fastq) files were processed by the Cell Ranger v.6 toolkit for sample demultiplexing, barcode processing, and gene counting as described previously (Nguyen T, et al. Front Bioeng Biotechnol. 2022 10:914450). Here, the reads were only aligned with the Pig Sscrofa11.1 genomes. The percentage of read mapping to the reference genome, a key indicator of sequencing quality, was above 90% in all samples. Barcodes were removed if they had fewer than 500 UMIs, more than 30000 UMIs, or >5% mitochondrial UMIs. DoubletFinder (McGinnis C S, et al. Cell Syst. 2019 8(4):329-37 e4) was applied to detect which barcodes could potentially be doublets. Then, the individual-animal data were integrated and normalized by the Seurat toolkit (Hao Y, et al. Cell. 2021 184(13):3573-87 e29) Merge and ScaleData functions with vars.to.regress set to nUMI and nGenes. Then, the expression was log-transformed (base 2) and exported into the sparse-matrix format.

Then, the Autoencoder toolkit (Nguyen T, et al. Scientific Reports. 2023 13(1):6821), embedded the high-dimensional (25880 genes) expression data into just 10 dimensions for each cell. After embedding, the results were visualized via Uniform Manifold Approximation (UMAP) (McInnes L, et al. arXiv preprint arXiv:180203426. 2018). Clustering was performed with the UMAP density-based clustering (DBSCAN) algorithm. Cell-type specificity of clusters was assigned based on the strongly upregulated expression of markers for the corresponding cell lineages (cardiomyocytes: Actin Alpha Cardiac Muscle 1 [ACTC1], Myosin Heavy Chain 7 [MYH7], Troponin T2 Cardiac Type [TNNT2], and Cardiac Muscle Ryanodine Receptor-Calcium Release Channel [RYR2]; cardiac endothelial cells: Platelet And Endothelial Cell Adhesion Molecule 1 [PECAM1]; cardiac smooth muscle cells: Gap Junction Protein Gamma 1 [GJC1] and Smooth Muscle Actin Alpha 2 [ACTA2]; monocytes-macrophages: Macrophage-Associated Antigen [CD163]; lymphocytes: CD3 Epsilon Subunit Of T-Cell Receptor Complex [CD3E], CD3 Gamma Subunit Of T-Cell Receptor Complex [CD3G], and T-Cell Surface Glycoprotein CD8 Alpha Chain [CD8A]; cardiac fibroblasts: Collagen Type I Alpha 1 Chain [COL1A1] and Fibronectin 1 [FN1]).

DoubletFinder (McGinnis C S, et al. Cell Syst. 2019 8(4):329-37 e4), the doublet detection method, is sensitive in labeling cells expressing multiple cell-type markers as ‘doublet’. However, under stress, such as injury and proliferation, one cell type may perform specific transitions, such as the mesenchymal-endothelial transition (Kovacic J C, et al. J Am Coll Cardiol. 2019 73(2):190-209), which are important cellular responses. During these transitions, the cells may express marker genes of other cell types and form a cluster in the snRNAseq data; hence, DoubletFinder might misclassify these cells as ‘doublet’. To handle this issue, we referred to human-induced pluripotent stem cell-derived cardiomyocyte expression in (Wang L, et al. Front Bioeng Biotechnol. 2023 11:1108340), which represented a ‘pure’ cardiomyocyte RNA data. In this data set, the expression of cardiac endothelial cell (PECAM1), macrophage (CD163), and lymphocyte (CD3E) were below the minimum expression threshold (100 transcripts) (Wang L, et al. Front Bioeng Biotechnol. 2023 11:1108340); meanwhile, cardiac fibroblast (COL1A1) and smooth muscle cells (GJC1) markers expression passed the threshold level. Therefore, we examined small cell clustered being classified as ‘doublet’ by Doublet Finders, visualized the coexpression among cardiomyocyte markers (MYH7, ACTC1, and RYR20), endothelial cell markers, macrophage, and lymphocyte markers as in (Nguyen T, et al. Front Bioeng Biotechnol. 2022 10:914450), then removed cells coexpressing at least two of these four cell types markers.

Then, the cardiomyocyte cell-cycle-specific gene expression was extracted. The cell-cycle specific gene list, which comprised 1646 genes, was obtained from the cycle Gene Ontology Term′ (GO:0007049). A cell-cycle specific autoencoder was computed using these genes. This Autoencoder has three computing layers: an input layer of 1646 nodes representing each cell's original cell cycle expression, a hidden (embedded) layer of 10 nodes, and an output layer of 1646 nodes representing the synthetic cell cycle expression. This cell-cycle-specific Autoencoder was computed as in (Nguyen T, et al. Front Bioeng Biotechnol. 2022 10:914450; Nguyen T, et al. Scientific Reports. 2023 13(1):6821). Then, cardiomyocytes were visualized and clustered by the UMAP toolkit, as described above.

On the other hand, the day-80 embryonic (Fetal) and postnatal-day-56 naïve-heart (CTL-P56) pig cardiomyocyte snRNAseq dataset was integrated to apply the sparse model analysis (Nguyen T, et al. Front Bioeng Biotechnol. 2022 10:914450; Nguyen T, et al. Scientific Reports. 2023 13(1):6821; Nakada Y, et al. Circulation. 2022 145(23):1744-7). Seven signaling pathways that were known to be important in cardiac regeneration (Zhang E, et al. PLoS One. 2020 15(7):e0232963; Singh B N, et al. Nat Commun. 2018 9(1):4237; Kawagishi H, et al. J Mol Cell Cardiol. 2018 123:180-4; Peng Y, et al. Front Cell Dev Biol. 2021 9:632372) (MAPK, HIPPO, cAMP, JAK-STAT, Hedgehog, TGFβ and RAS) were analyzed. The calculation is as follows: 1) for each pathway, the sparse support vector machine (SVM) only used genes belonging to the pathway to classify between the Fetal and CTL-P56 cardiomyocytes (baselines); 2) the SVM was optimized to score and archive the best baseline classification accuracy, where SVM is expected to give high scores (>2) for most of Fetal cardiomyocytes and low scores (<1) for most of CTL-P56 cardiomyocytes; 3) then, the SVM, which was specific for the pathway, was applied to score other groups' cardiomyocyte. After the SVM calculation, the ‘sparse model score’ is defined as the proportion of cardiomyocytes with SVM score >2 in each group. Pathways reaching the minimum of 2-fold higher in SP+I/R-1 week, compared to I/R-1 week group, were selected. Then, these pathways' growth factors were examined in: 1) the snRNAseq dataset for whether they expressed in at least 1% of the cells, and 2) our previously published hiPSC-CM dataset (Wang L, et al. Front Bioeng Biotechnol. 2023 11:1108340) for they had Fragments Per Kilobase of transcript per Million (FPKM) of 1.0 and above.

Statistics

Data was calculated and expressed as Mean±SEM. Statistical analysis was performed using Prism. The difference between two groups was tested for significance using independent T-test. Comparisons among groups were analyzed for significance with one-way analysis of variance (ANOVA) with Tukey correction. A value of p<0.05 was considered significant. Non-parametric Wilcoxon's Ranksum test was performed on snRNAseq data, where the data was not always normally distributed.

Results

Generation and Characterization of ^{HLA-KO/CCND2-OE}hiPSC and ^{HLA-KO/CCND2-OE}hiPSCs Derived CMs

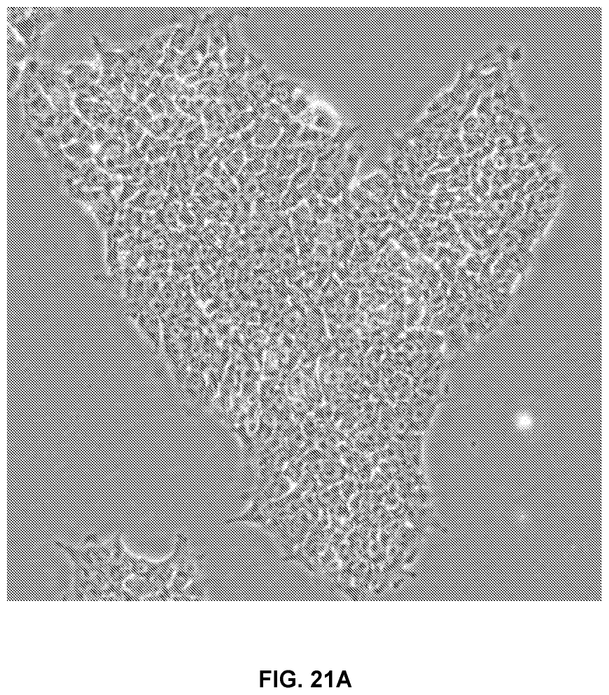

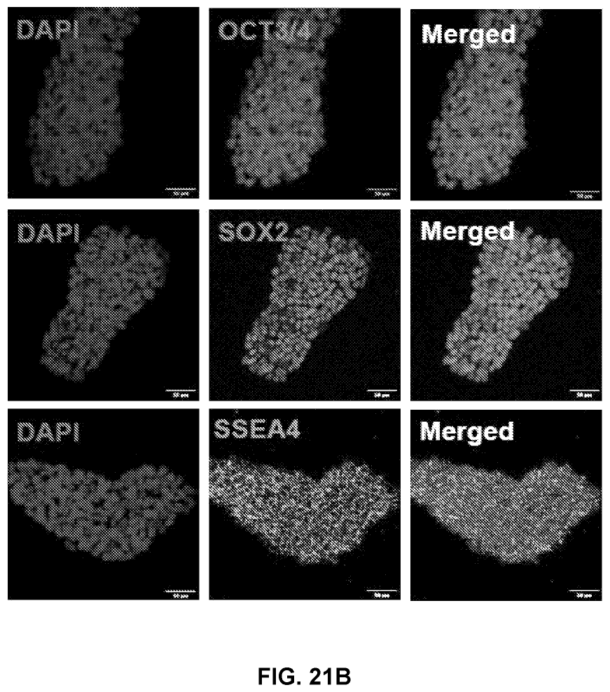

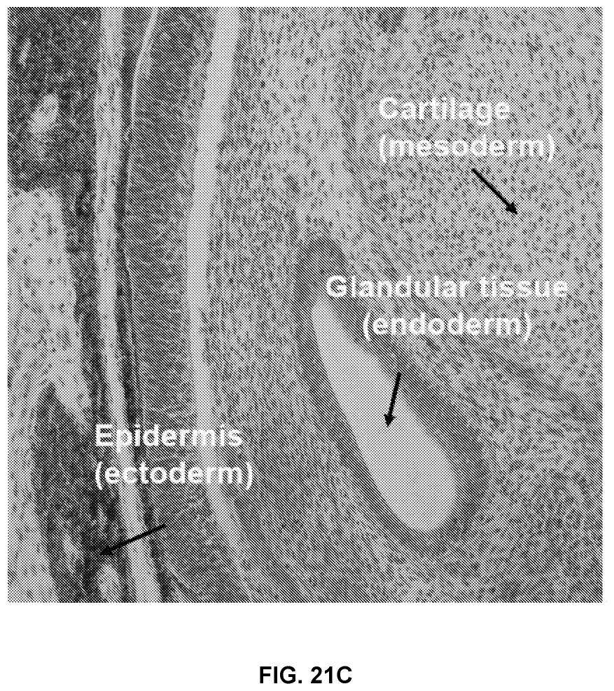

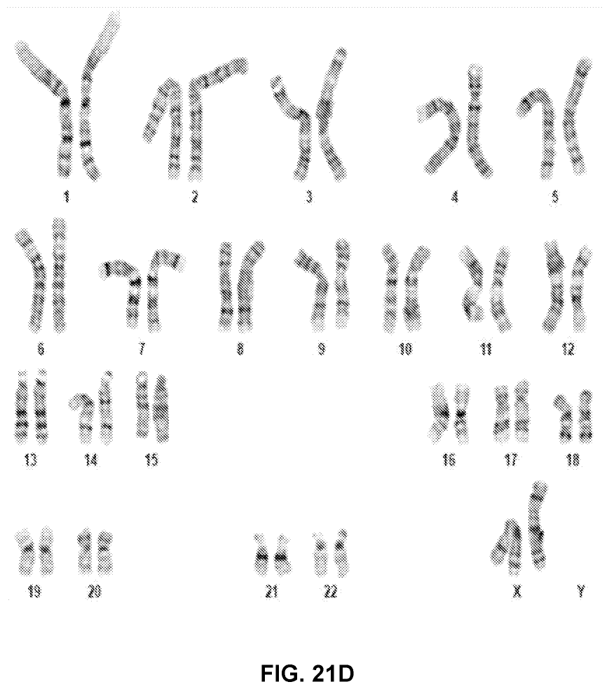

A hiPSC line with HLA-I and -II knockout and MHC driven CCND2 overexpressing was created (FIG. 21). The quality of ^{HLA-KO/CCND2-OE}hiPSC s was checked visually: a homogeneous appearing colony with clear border. They expressed pluripotency markers, including OCT4, SOX2, and SSEA4 and formed teratoma in SCID-NOD mice (FIGS. 21A-21C). Karyotyping analysis confirmed that ^{HLA-KO/CCND2-OE}hiPSC s had normal human chromosomal structure and numbers (FIG. 21D).

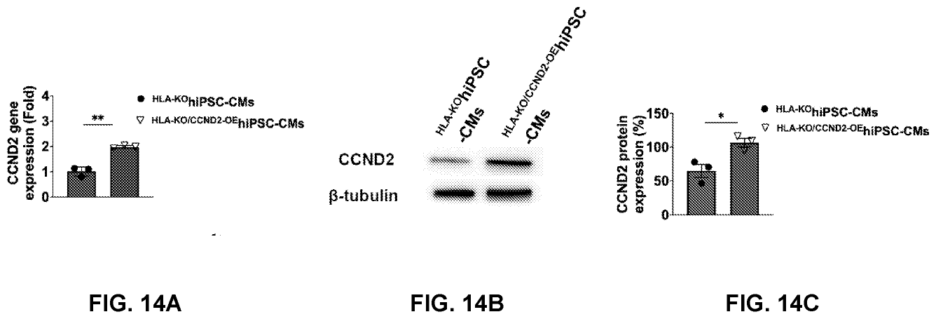

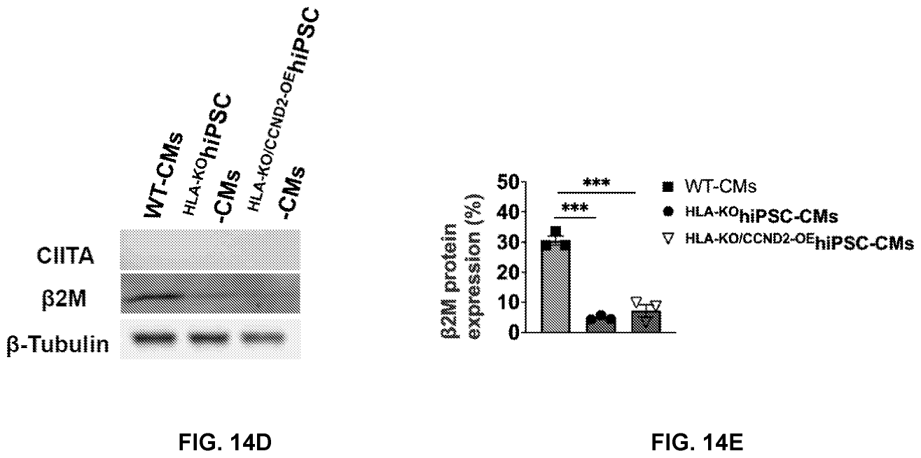

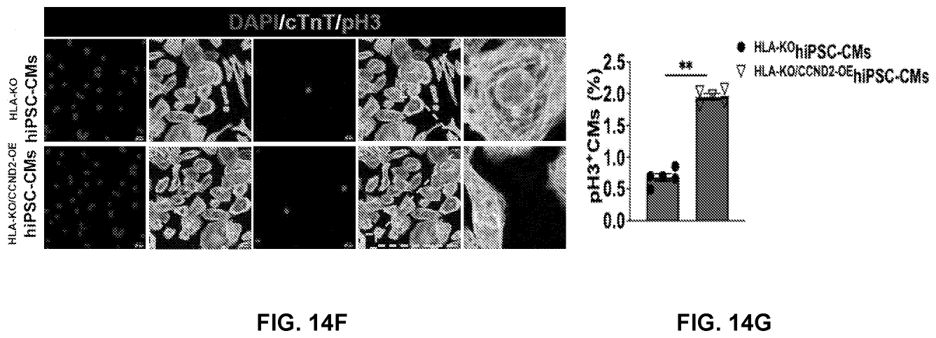

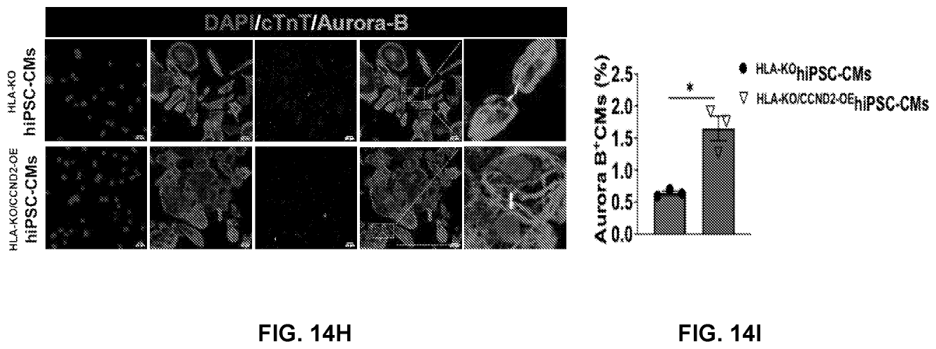

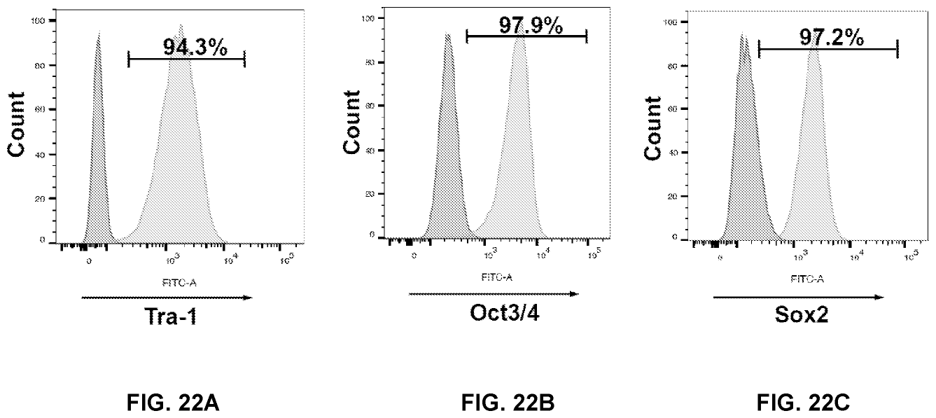

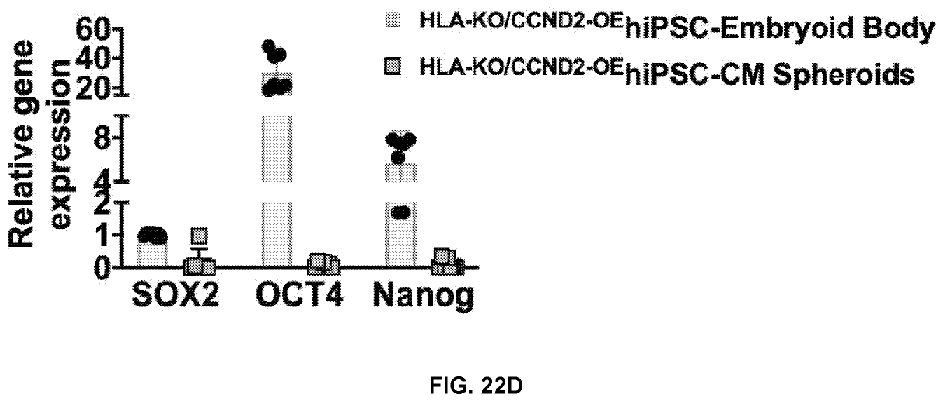

To determine the protein expression levels of HLA-I, -II and CCND2, hiPSCs were differentiated into CMs (FIG. 22). Gene and protein expression levels of CCND2 in ^{HLA-KO/CCND2-OE}hiPSC derived CMs (^{HLA-KO/CCND2-OE}hiPSC-CMs) were significantly higher than those in ^{HLA-KO}hiPSC derived CMs (^{HLA-KO}hiPSC-CMs) (FIGS. 14A-140). On the contrary, the protein expression level of 82-microglobulin was significantly reduced in both ^{HLA-KO}hiPSC-CMs and ^{HLA-KO/CCND2-OE}hiPSC-CMs as compared with wild type hiPSCs derived CMs (^{WT}hiPSC-CMs) (FIGS. 14D & 14E), while the protein expression of HLA class II transactivator (CIITA) was almost undetectable in all three hiPSC-CMs regardless knockout (FIG. 14D).

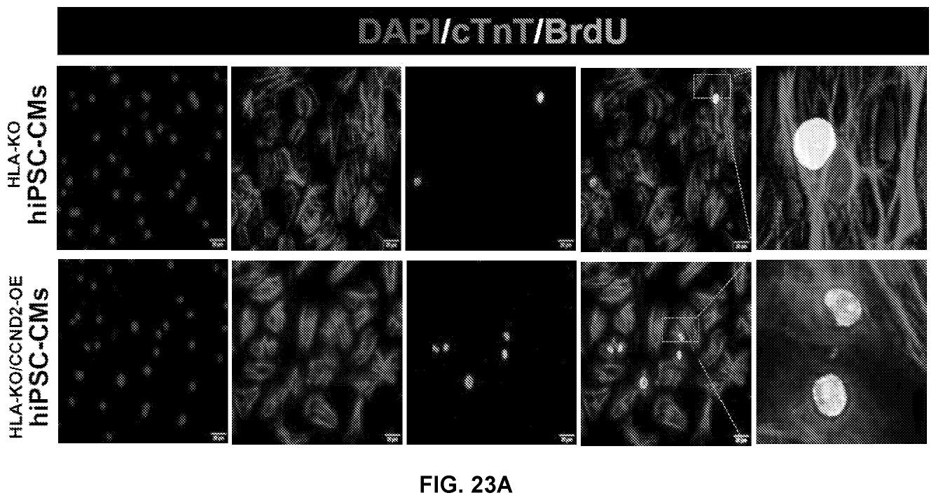

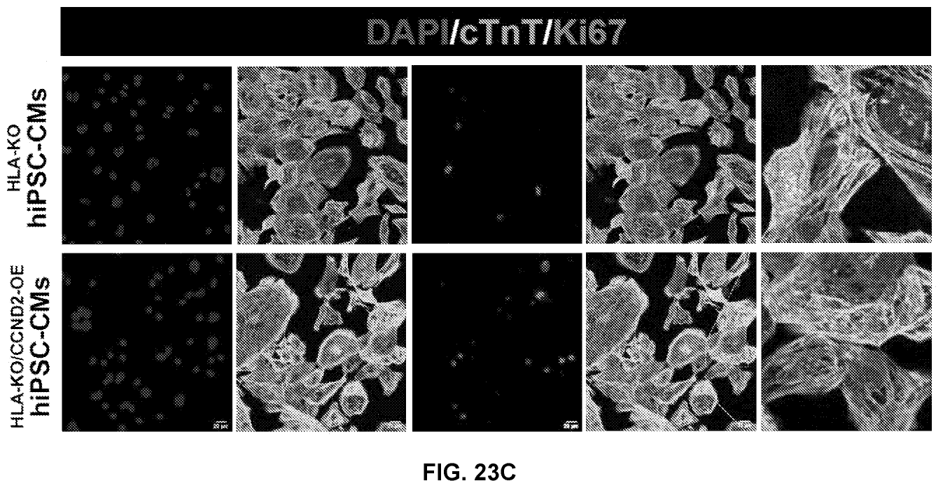

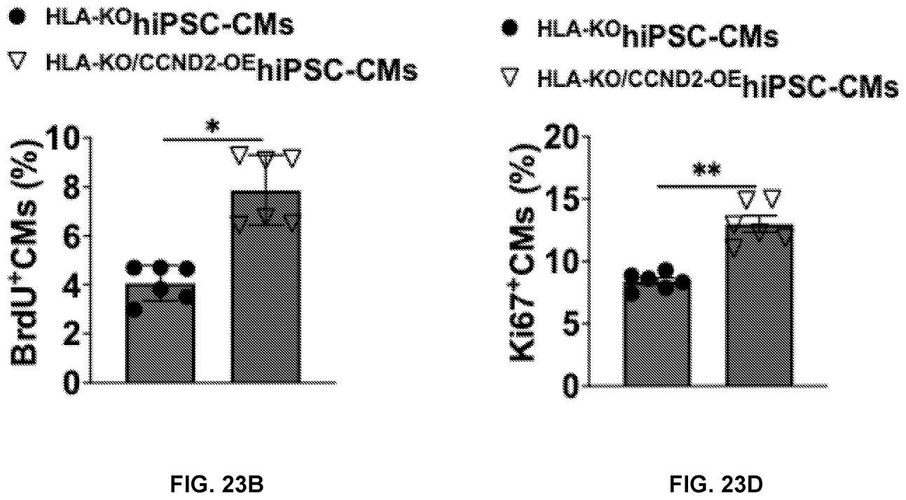

To determine whether CCND2-Overexpression could promote the proliferation in hiPSC-CMs, ^{HLA-KO/CCND2-OE}hiPSC-CMs was immunostained for BrdU, a DNA synthesis marker [57, 58], Ki67 and phosphorylated histone 3 (pH3)[59, 60], mitosis markers, and Aurora B, a cytokinesis marker (Engel F B, et al. Genes Dev. 2005 19(10):1175-87; Bersell K, et al. Cell. 2009 138(2):257-70). BrdU staining indicated that more DNA synthesis were observed in ^{HLA-KO/CCND2-OE}hiPSC-CMs than ^{HLA-KO}hiPSC-CMs (FIGS. 23A & 23B). Consistently, the number of Ki67⁺ or pH3^{+HLA-KO/CCND2-OE}hiPSC-CMs was significantly higher than ^{HLA-KO}hiPSC-CMs (FIGS. 23C & 23D and FIGS. 14F & 14G). The number of Aurora B⁺ CMs under cytokinesis also significantly increased in ^{HLA-KO/CCND2-OE}hiPSC-CMs compare to the ^{HLA-KO}hiPSC-CMs (FIGS. 14H & 14I). These data suggest that ^{HLA-KO/CCND2-OE}hiPSC-CMs have significantly increased mitosis and cytokinesis as compared with ^{HLA-KO}hiPSC-CMs.

Large Quantity of ^{HLA-KO/CCND2-OE}hiPSC-CMs Generated Using Spheroid Differentiation Method

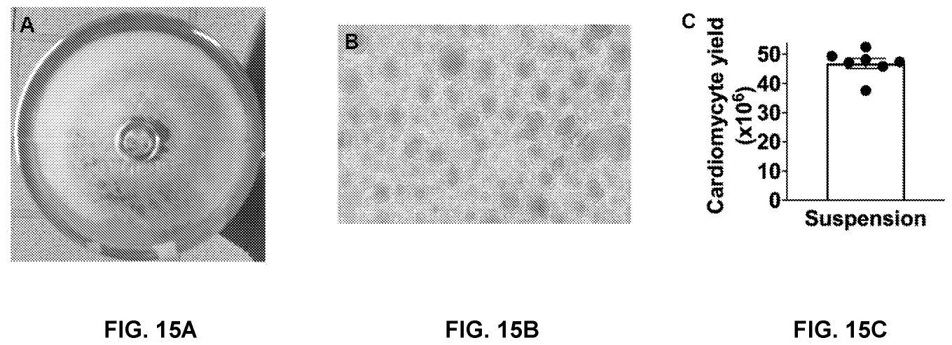

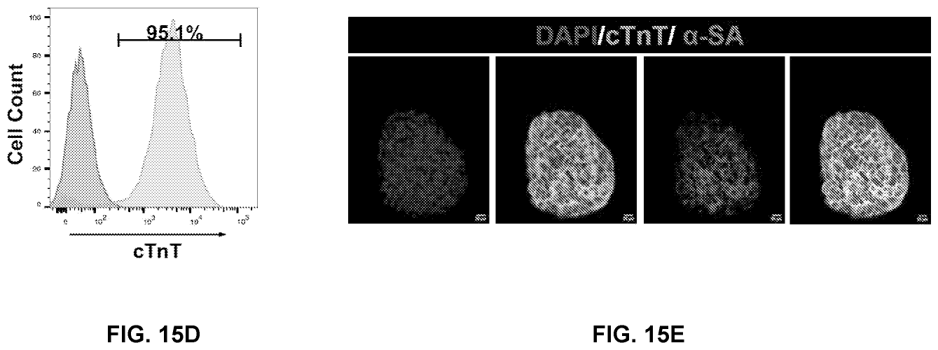

hiPSC-CM spheroids were generated and purified (FIG. 15A). The mean diameter of CM spheroids was 231.6±57.7 on day 12 after purification (FIG. 15B). Spontaneous contraction was observed in the spheroids on day 12 after differentiation. The suspension differentiation generated 46.9±4.6 million of hiPSC-CM with purity 95% each batch (FIGS. 15C & 15D). hiPSC-CMs in spheroids co-expressed cTnT and α-sarcomeric actinin (α-SA) (FIG. 15E).

Implantation of ^{HLA-KO/CCND2-OE}hiPSC-CM Spheroids Enhances Cardiac Function and Increases Cardiomyocytes Proliferation after I/R in Swine

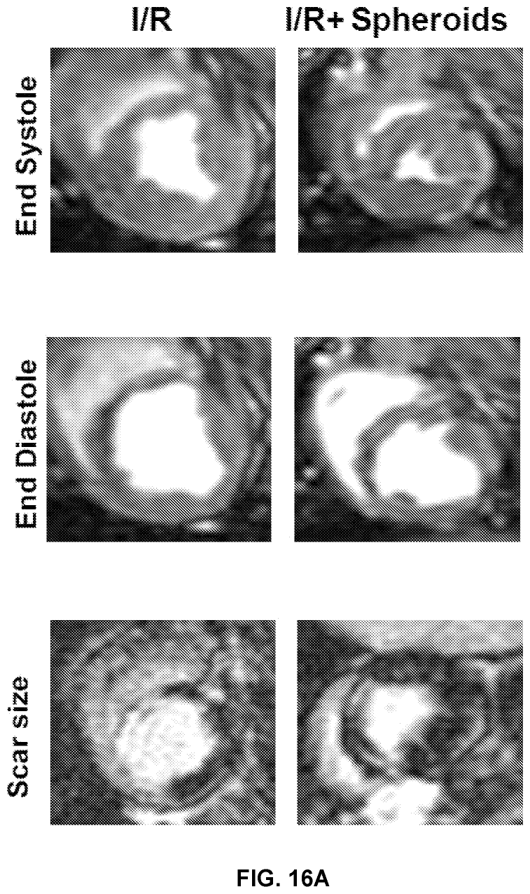

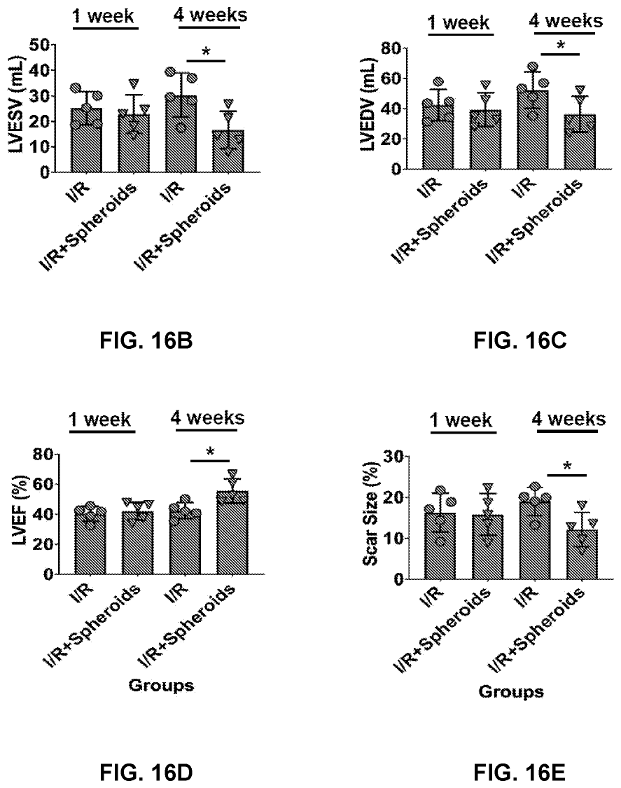

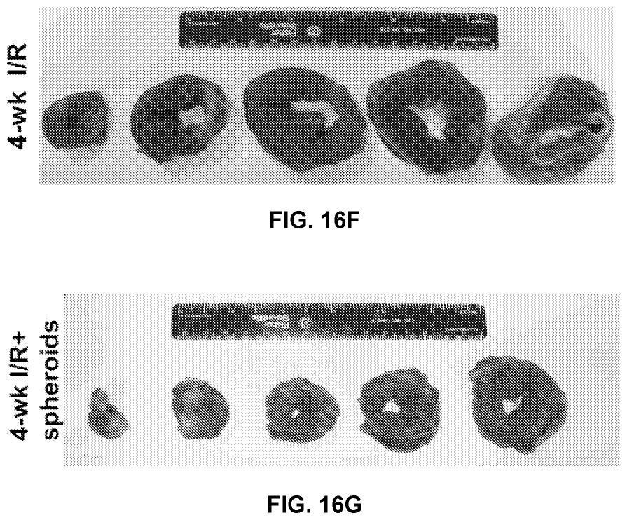

We next assessed therapeutic efficacy of ^{HLA-KO/CCND2-OE}hiPSC-CM spheroids for the treatment of myocardial injury in a clinically relevant large animal model, pig heart models of I/R. cMRI showed that both the LVEDV and LVESV were significantly smaller in the spheroid Group than the I/R Group at 4 weeks after injury and treatment (p<0.01 for both, FIGS. 16A-16C). Consistently, the LVEF significantly improved in the spheroid Group as compared to the I/R Group at 4 weeks after injury and treatment (p<0.01, FIG. 16D). In addition, spheroid transplantation significantly reduced infarct size (FIG. 16E) as compared with the I/R Group (p<0.01), which was confirmed by gross tissue imaging showed that spheroid transplantation resulted in much less fibrotic tissue in the pig hearts than in the pig hearts of the I/R Group (FIGS. 16F & 16G).

Engraftment of ^{HLA-KO/CCND2-OE}hiPSC-CM in the Pig Hearts

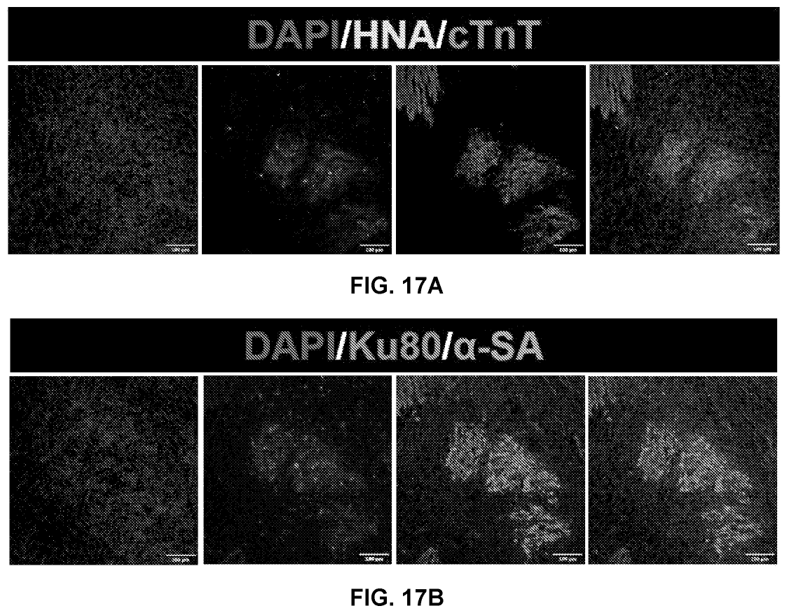

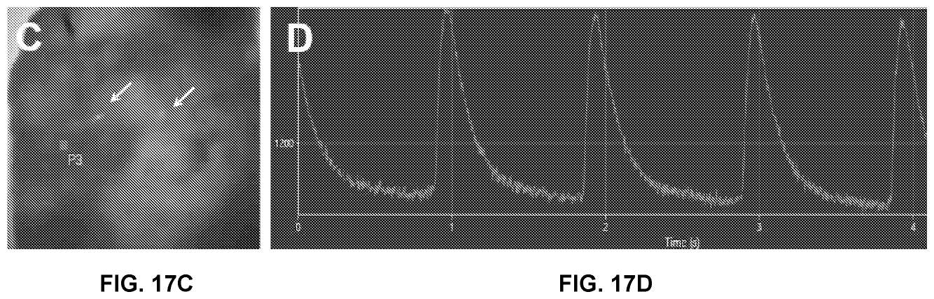

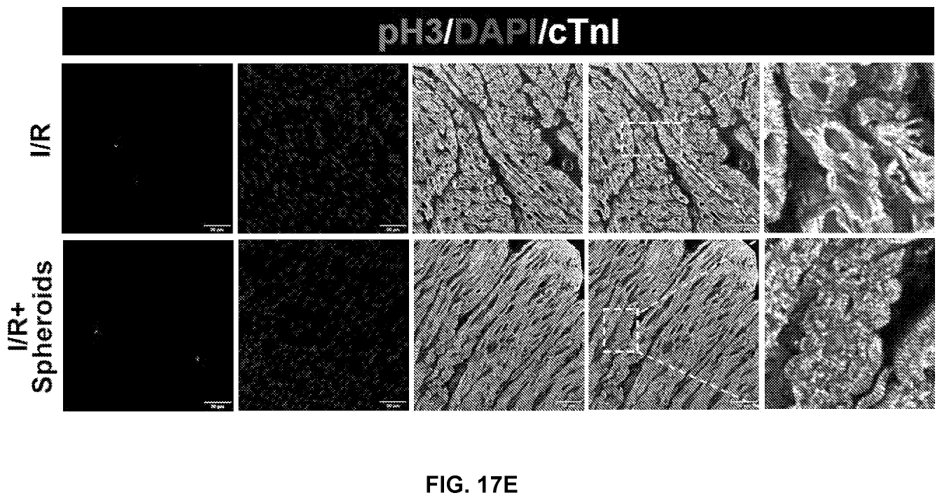

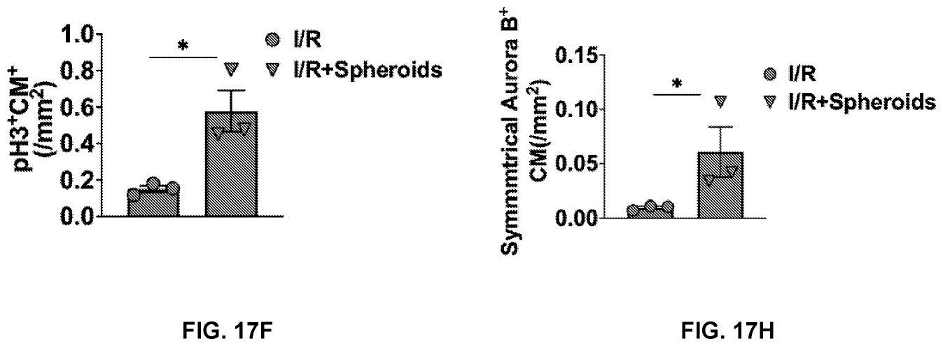

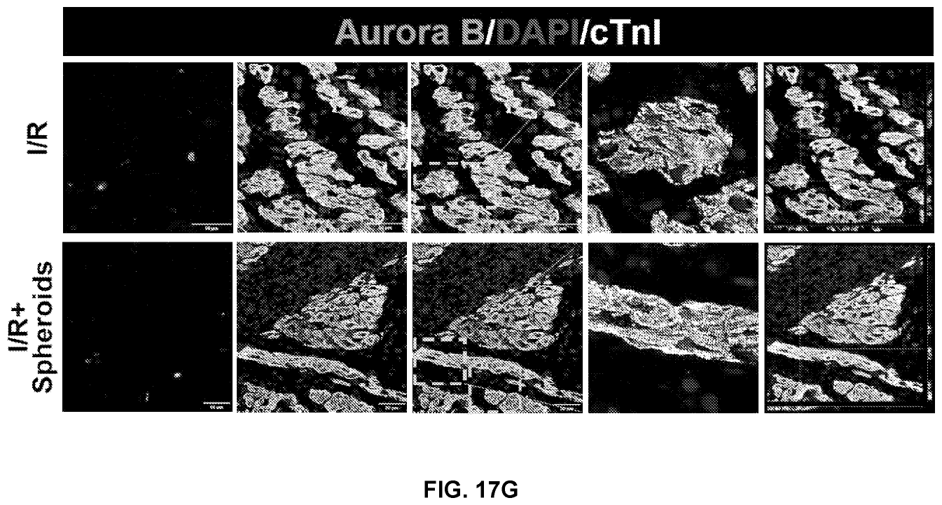

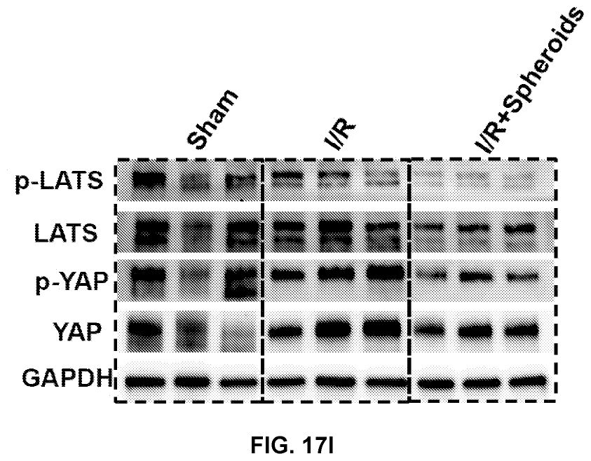

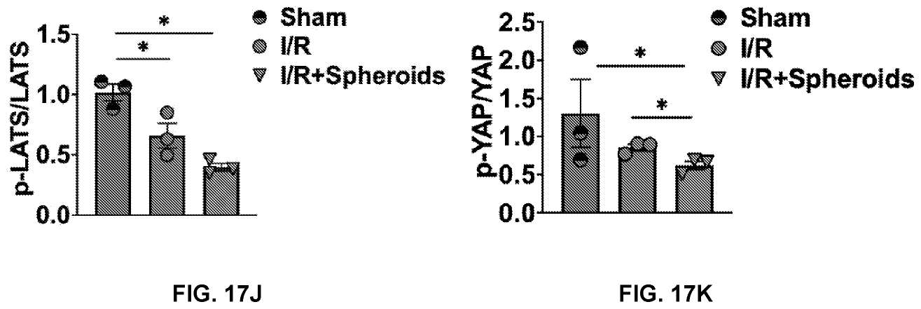

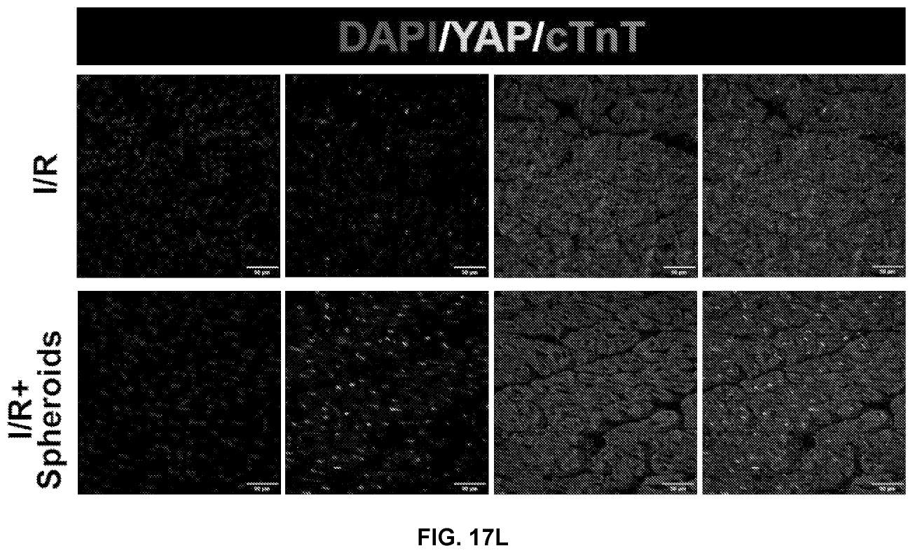

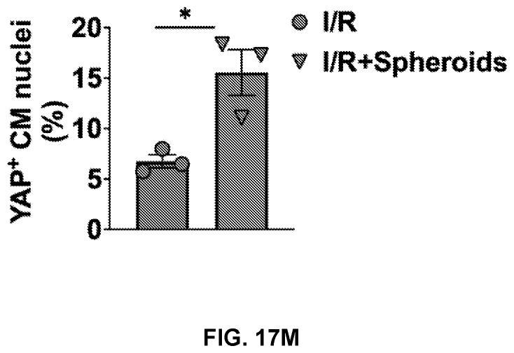

The engraftment of ^{HLA-KO/CCND2-OE}hiPSC-CMs was assessed using human-specific nuclear antigen (HNA, FIG. 17A) (Gerdes J, et al. Int J Cancer. 1983 31(1):13-20) or human nuclear marker (Ku80, FIG. 4B) (Reidling J C, et al. Stem Cell Reports. 2018 10(1):58-72) together myocyte specific markers. Immunofluorescence staining of cryo-sections showed that cells stained positive for HNA also co-expressed cTnT (FIG. 17A) and cells also co-expressed Ku80 and α-SA (FIG. 17B) in one-week pig heart tissues. However, no engraftment of ^{HLA-KO/CCND2-OE}hiPSC-CM was observed in the porcine hearts at 4 weeks after injury and treatment.

Optical mapping further confirmed that ^{HLA-KO/CCND2-OE}hiPSC-CMs survived in pig myocardium (FIGS. 17C & 17D) at 1 week after transplantation. The injected spheroids depicted calcium transient with bright flashes during optical mapping and CaT signal from the GCaMP6 transfected spheroids clearly showed that spheroids were alive.

^{HLA-KO/CCND2-OE}hiPSC-CM Transplantation Induced Porcine CM Proliferation

As both cMRI and gross tissue sections showed significantly less fibrotic tissue in ^{HLA-KO/CCND2-OE}hiPSC-CM transplanted pig hearts, we investigated whether endogenous porcine CM proliferation contributed to porcine heart regeneration after I/R. We assessed mitosis (Ki67 and pH3) and cytokinesis (ABK) markers at the border zone (BZ) and remote zone (RZ) of injury in the porcine hearts.

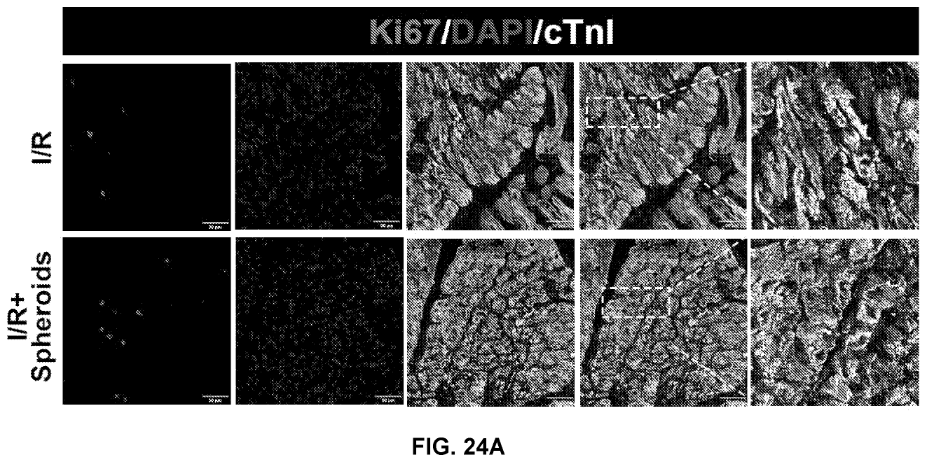

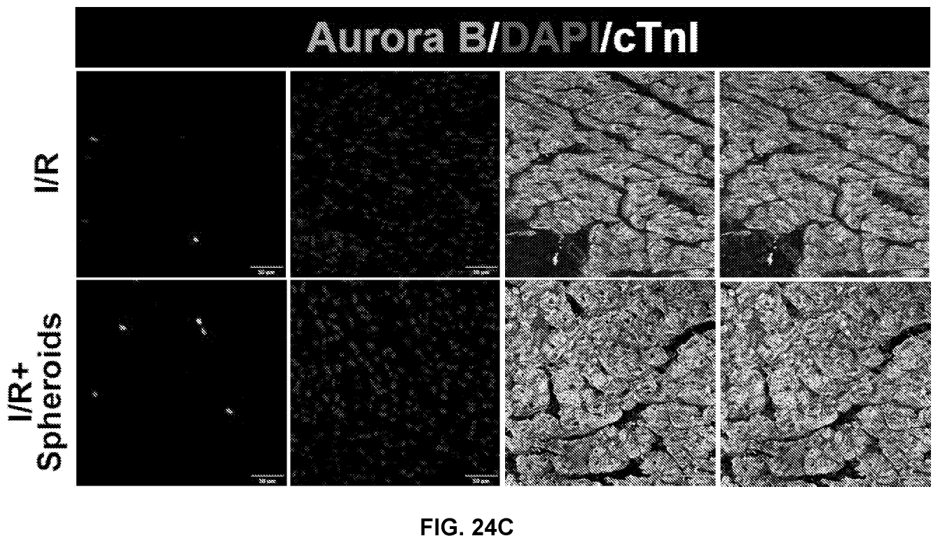

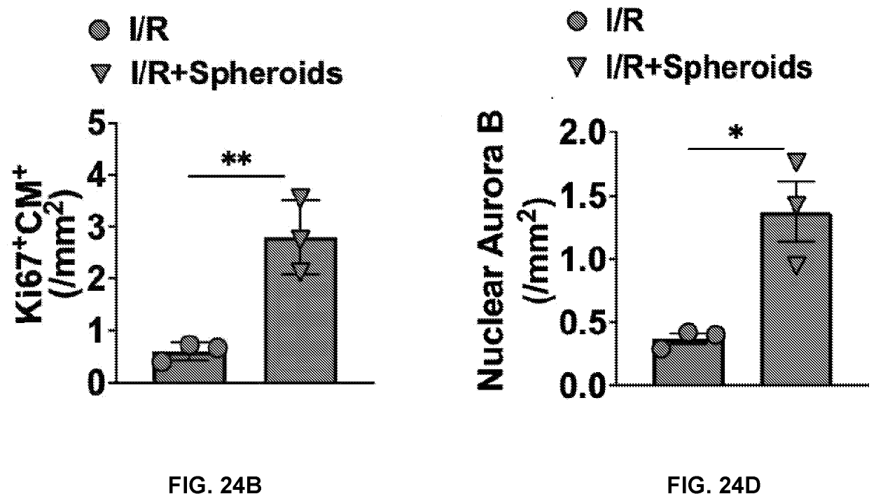

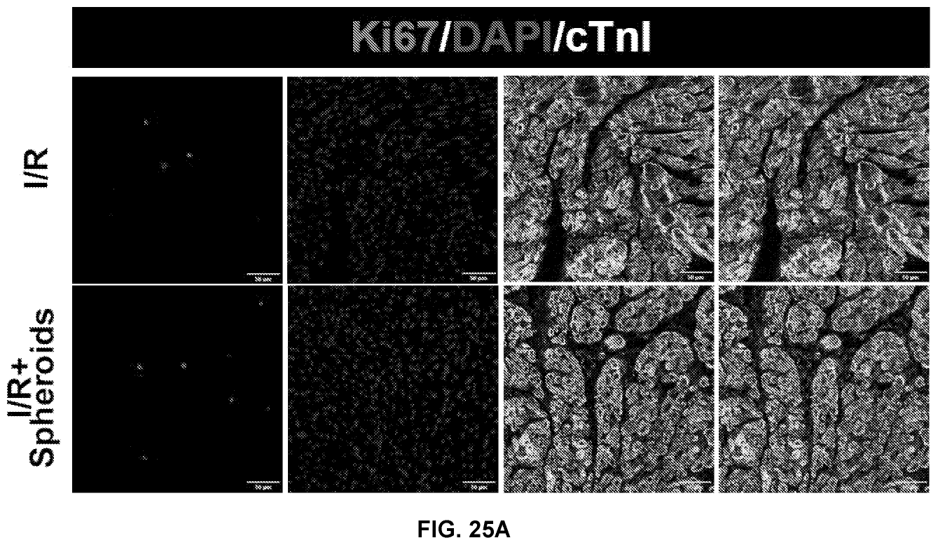

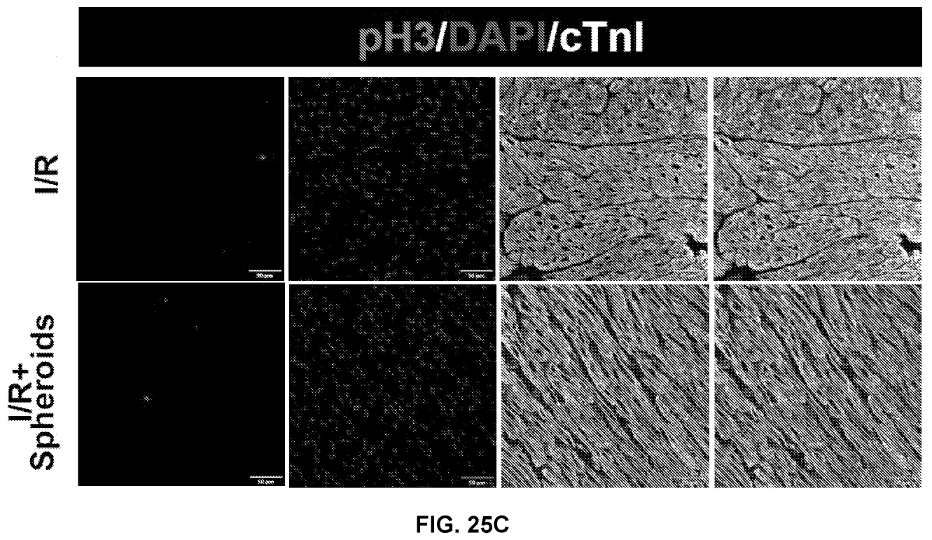

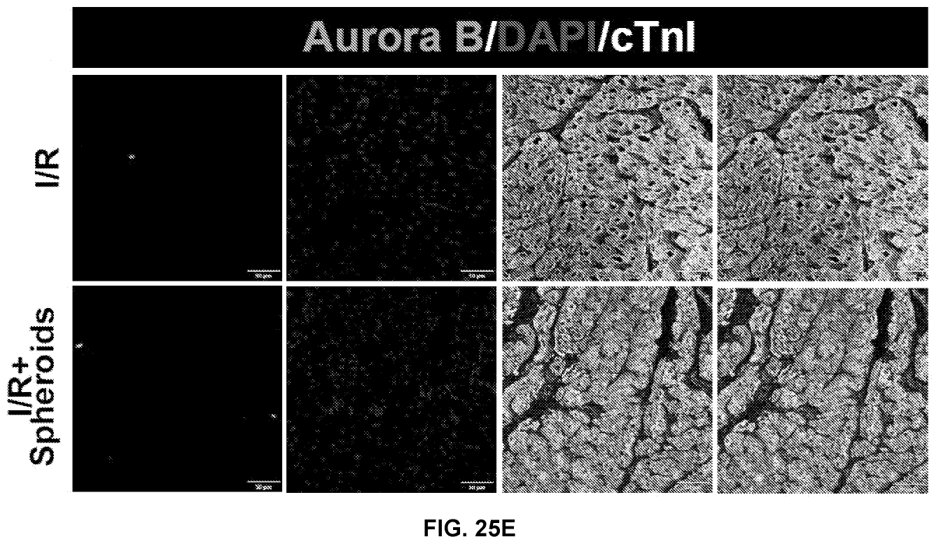

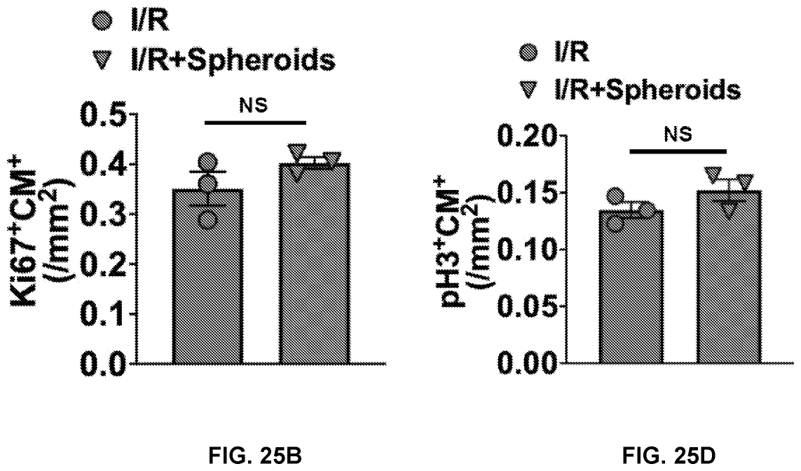

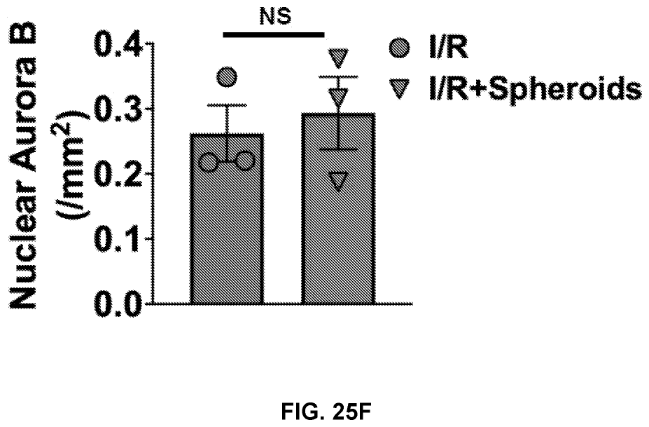

At one-week after injury and treatment, both the numbers of Ki67⁺ (FIGS. 24A & 24B) and pH3⁺ (FIGS. 17E & 17F) porcine CMs were significantly higher at the BZ in the pig hearts of the Spheroid Group than the I/R group. More importantly, the number of porcine CM under cytokinesis, which was defined as the expression of symmetric ABK with sarcomere disassembly, in the Spheroid Group is >6 folds of the I/R Group (FIGS. 17G & 17H). Similarly, asymmetric ABK⁺ porcine CMs in the Spheroid Group is >3 folds the I/R Group (FIGS. 24C & 24D). At the RZ, the number of Ki67⁺ or pH3⁺ porcine CMs was similar between the Spheroid and I/R Groups (FIGS. 25A-25D). Although ABK⁺ porcine CMs were found at RZ, they were all asymmetric and similar between the Spheroid and I/R Groups (FIGS. 25E & 25F).

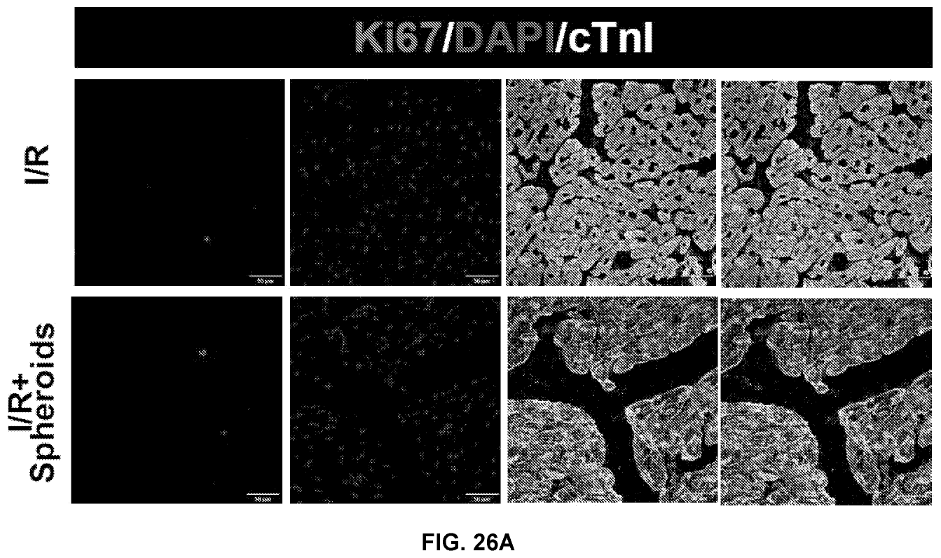

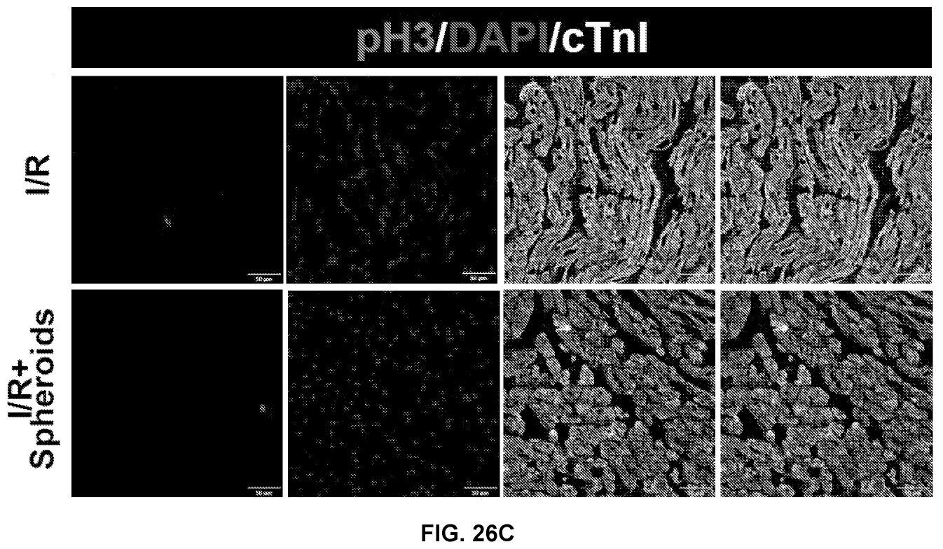

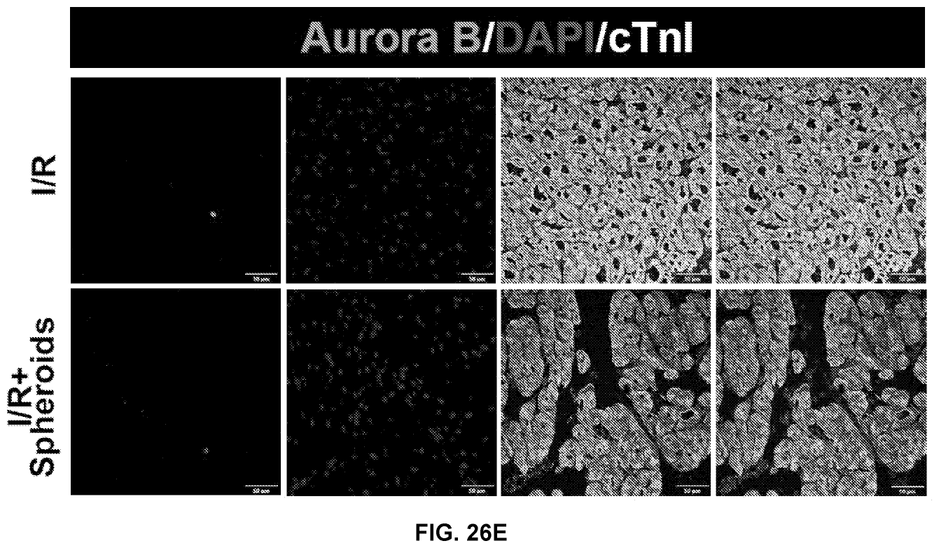

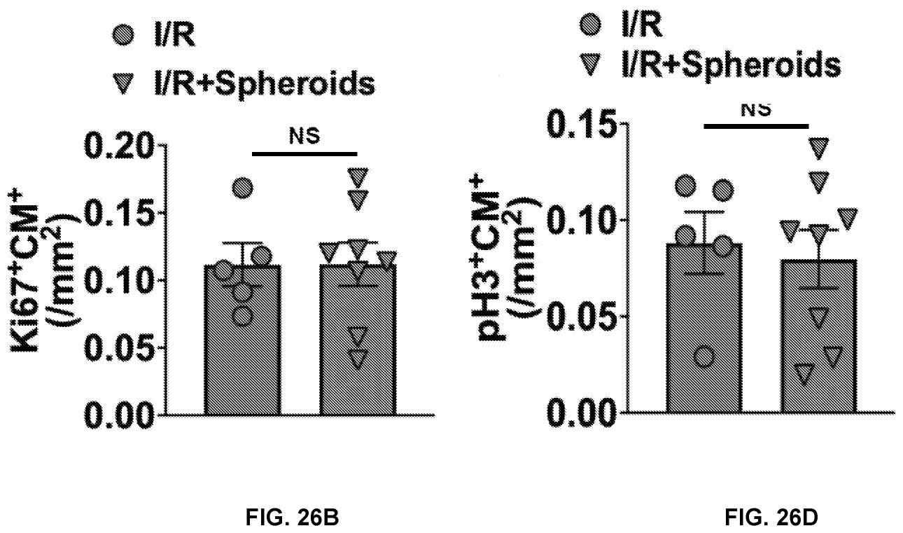

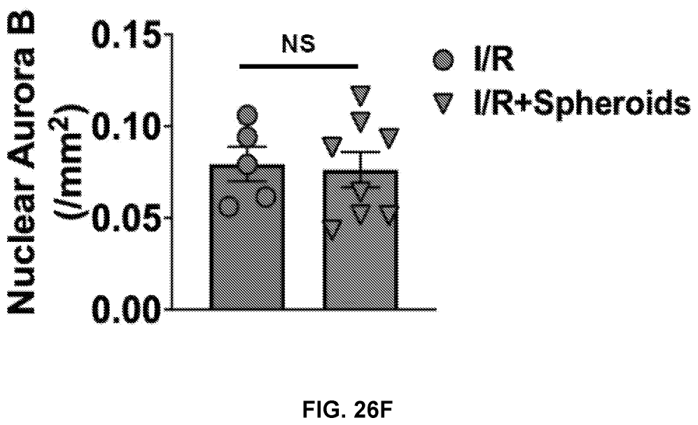

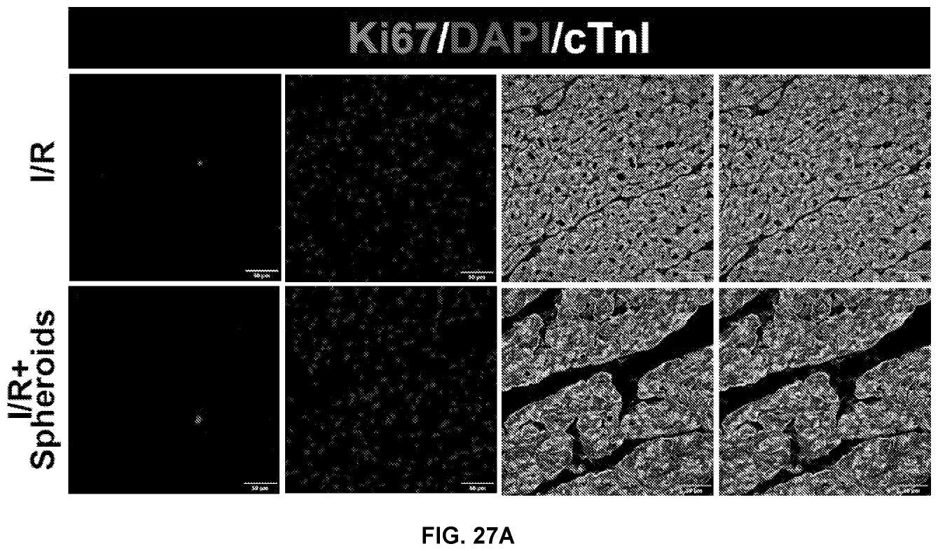

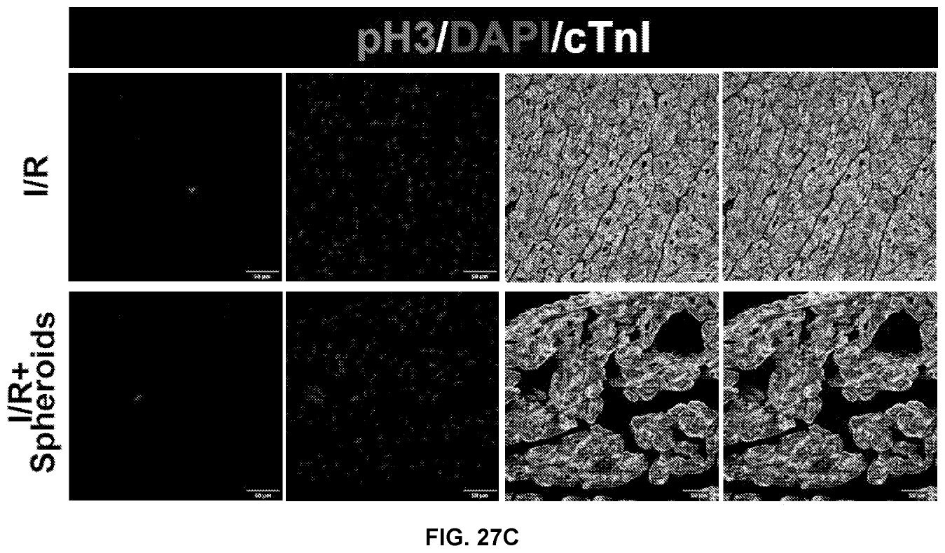

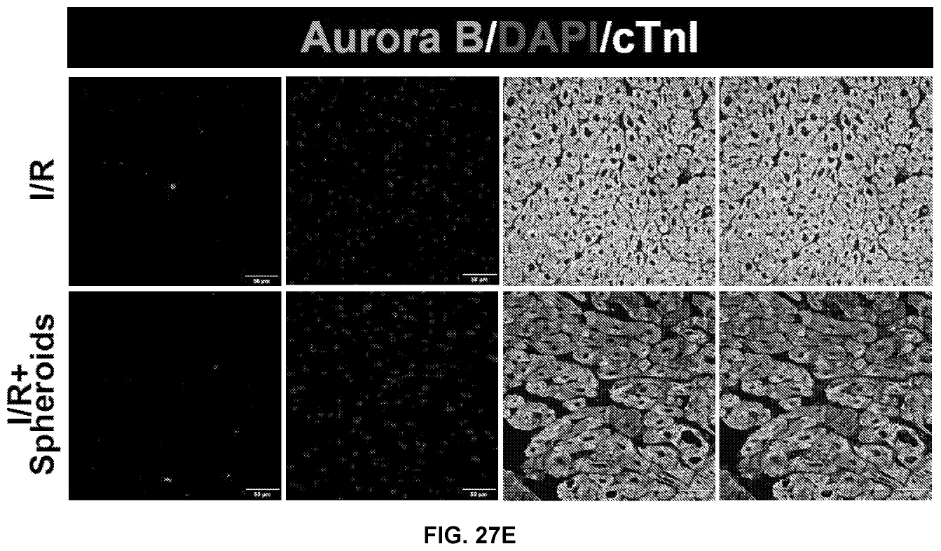

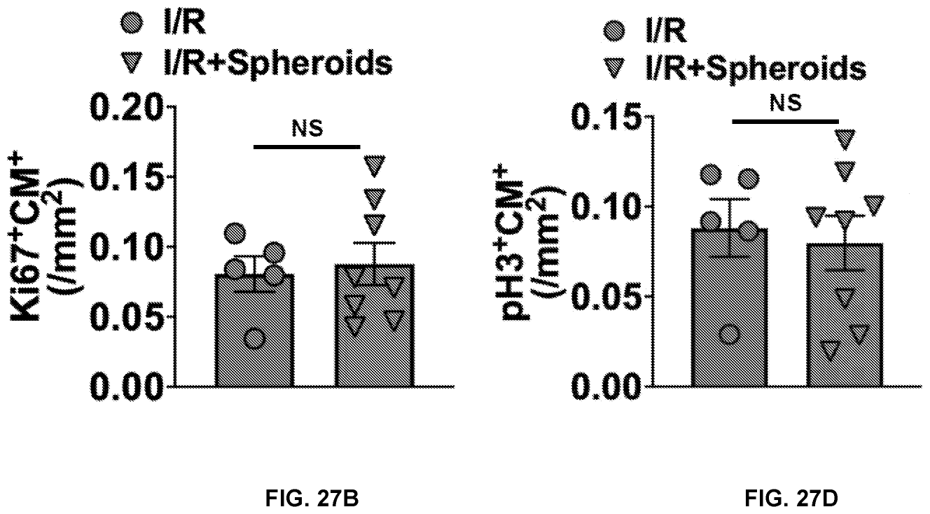

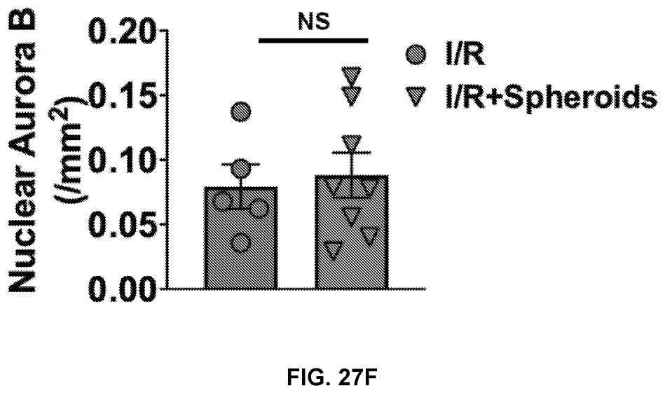

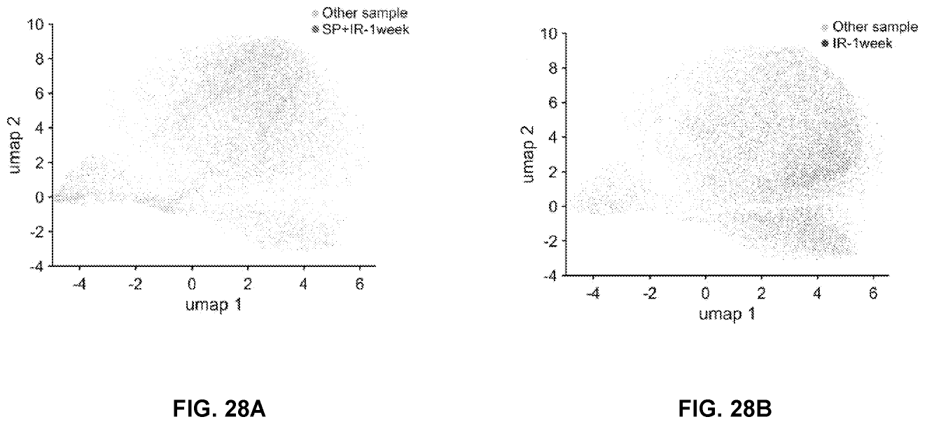

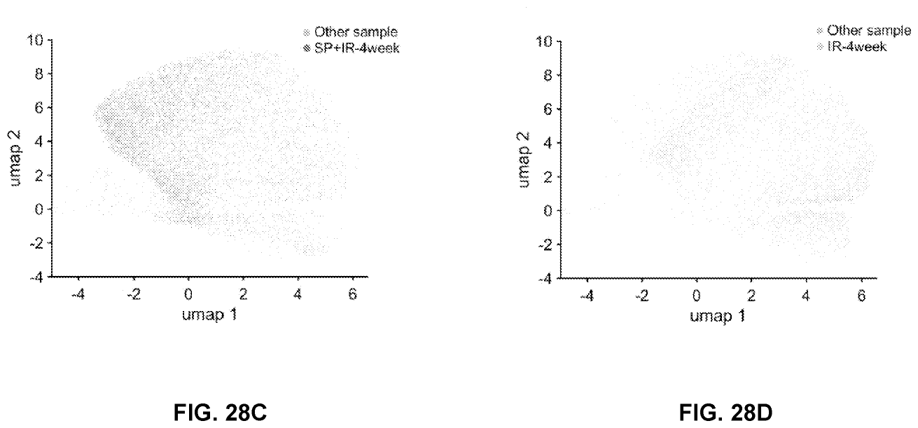

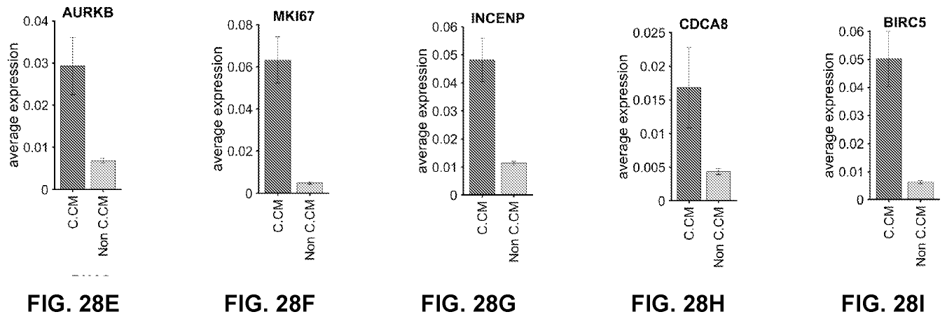

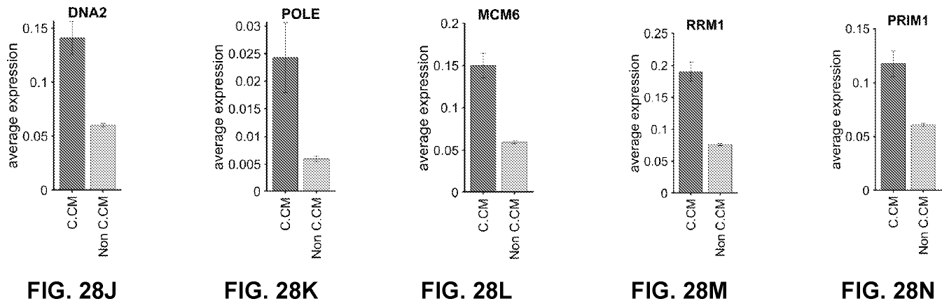

At four-week after injury and treatment, the numbers of Ki67⁺ (FIGS. 26A & 26B), pH3⁺ (FIGS. 26C & 26D), and asymmetric ABK⁺ (FIGS. 26E & 26F) porcine CMs were similar at the BZ in the pig hearts of the Spheroid and I/R groups. Similarly, the numbers of Ki67⁺ (FIGS. 27A & 27B), pH3⁺ (FIGS. 27C & 27D), and asymmetric ABK⁺ (FIGS. 27E & 27F) porcine CMs were also similar at the RZ in the pig hearts of the Spheroid and I/R Groups.

To determine whether the increased porcine CM proliferation at 1 week after spheroid injection was contributed by YAP signaling pathway, which is well known for inducing CM proliferation, Western blot analysis using BZ samples from the Sham, I/R, and Spheroid Groups was performed (FIG. 17I). The Spheroid Group had the lowest expression levels of pLATS1 (FIGS. 17I & 17J) and pYAP (FIGS. 17I & 17K). Thus, more non-phosphorylated YAP proteins are expected to be to nuclei where YAP activates genes responsible for cell proliferation (von Gise A, et al. Proc Natl Acad Sci USA. 2012 109(7):2394-9).

Fluorescence immunostaining for YAP protein expression showed that there was significantly higher number of porcine CM nuclei stained positively for YAP protein expression in the Spheroid Group as compared with the I/R Group (FIGS. 17L & 17M), which confirms that more YAP protein was transported to porcine CM nuclei. These data suggested that spheroid transplantation induced porcine CM proliferation may be achieved through modulating YAP signaling pathway.

^{HLA-KO/CCND2-OE}hiPSC-CM Transplantation Increased Porcine CM Cell-Cycle Activity as Shown by the snRNAseq Data

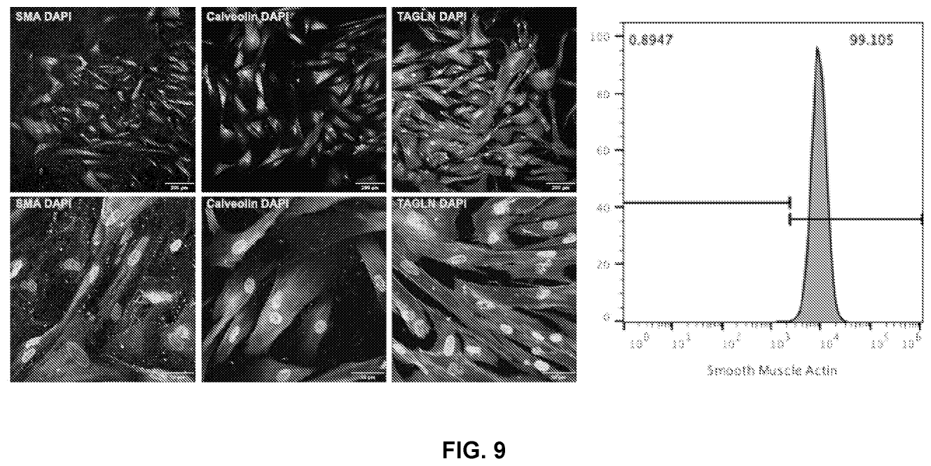

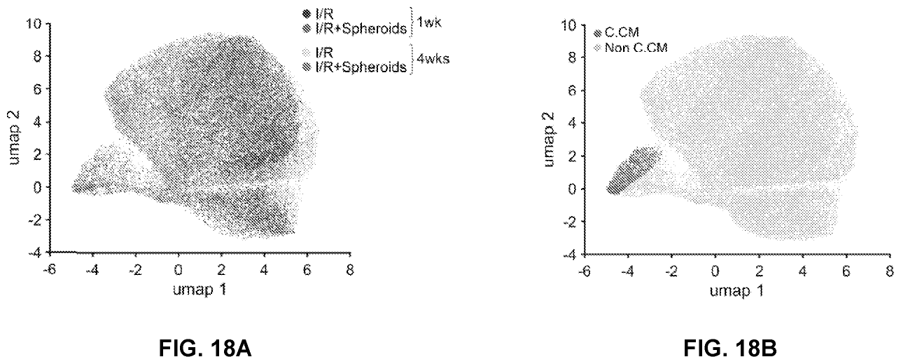

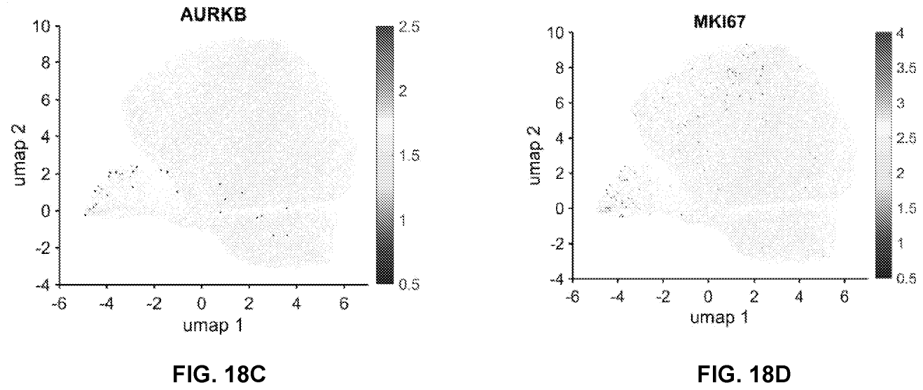

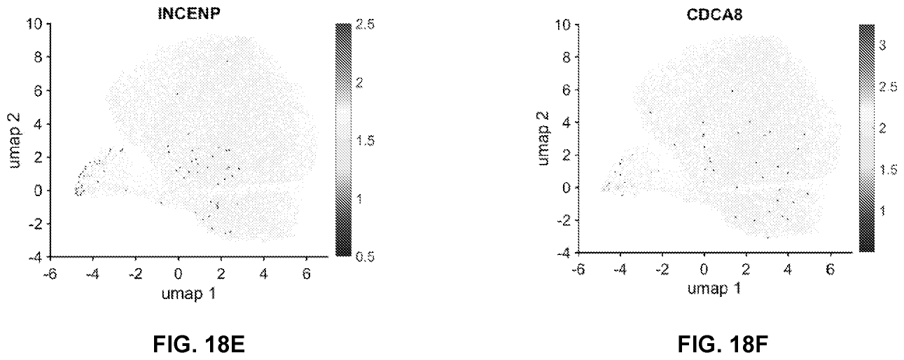

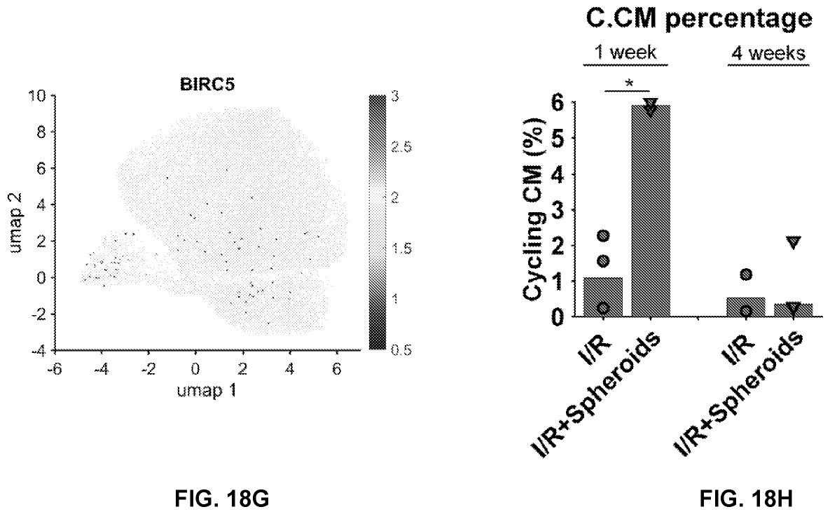

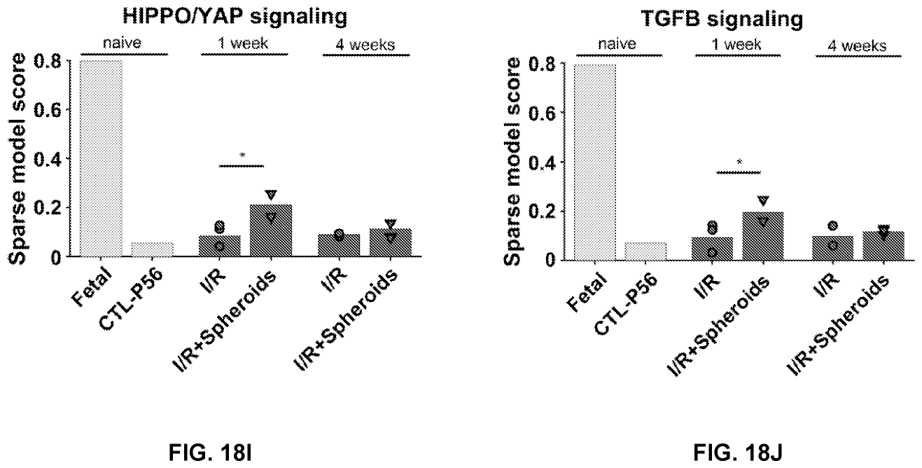

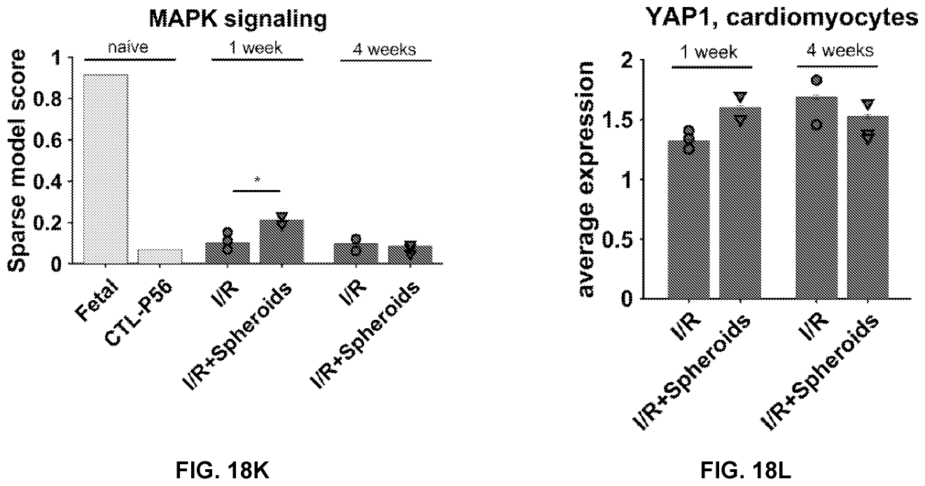

The snRNAseq data quality in all animals were shown in Supplemental Table 5, which were exported by the Cell Ranger v.6 toolkit. The cell-cycle-specific Autoencoder identified a cycling CM (C.CM) cluster, with negligible batch effect, from the snRNAseq data (FIGS. 18A & 18B, 28A-28D). The expression patterns of five proliferation markers: Aurora Kinase B [AURKB], Marker of Proliferation Ki-67 [MKI67], Inner Centromere Protein [INCENP], Baculoviral IAP Repeat Containing 5 [BIRC5], and Cell Division Cycle Associated 8 [CDCA8] colocalized in C.CM (FIGS. 5C-5G, Supplemental FIGS. 9E-9I). Furthermore, C.CM had upregulated genes involved in DNA replication process [66], notably DNA Replication Helicase/Nuclease 2 [DNA2], DNA Polymerase Epsilon, Catalytic Subunit [POLE], Minichromosome Maintenance Complex Component 6 [MCM6], Ribonucleotide Reductase Catalytic Subunit M1 [RRM1], and DNA Primase Subunit 1 [PRIM1] (Supplemental FIGS. 9J-9N). Based on the snRNAseq data, C.CM population was the highest (5.9%) in the Spheroid Group, which was >5 folds of that in the I/R Group (1.09%) at 1 week after I/R and treatment. The C.CM population only decreased to 0.53% and 0.35% in the I/R and Spheroid Groups at 4 weeks after I/R and treatment, respectively (FIG. 18H).

Pro-Proliferation Signaling Pathways Enriched in Porcine CMs in the Spheroid Group at 1 Week after I/R and Treatment

Seeing increased cell-cycle activity in porcine CMs of the Spheroid Group at 1 week after I/R and treatment, we performed enrichment analysis of pro-proliferation signaling pathways, which were previously identified in Zhang's study (Zhang E, et al. PLoS One. 2020 15(7):e0232963). Among seven pro-proliferation signaling pathways, the sparse model analysis identified the HIPPO/YAP, TGFβ, and MAPK pathways, where the enrichment scores (sparse model score) were significantly higher (>2 folds, p<0.05) in the Spheroid Group than the I/R Group (FIG. 18I-18K). Further analyzing YAP1 gene expression, there was a trend of increased YAP1 gene expression in the porcine CMs at 1 week after I/R and treatment (FIG. 18L). Other pathways, including Hedgehog, JAK-STAT, cAMP, and RAS signaling pathways, did not achieve a minimum increase of 2-fold in Spheroid Group, compared to I/R Group at 1 week after I/R and treatment.

There were 20 receptors associated with HIPPO/YAP, TGFβ, and MAPK pathways and were expressed by a minimum of 5% cardiomyocytes. Based on their interacting extracellular proteins which were queried via the STRING v.11.5 database (Szklarczyk D, et al. Nucleic Acids Res. 2023 51(D1):D638-D46), a total of 56 extracellular proteins were retrieved. Among these 56 extracellular genes, 17 of them were found expressed in at least 1% of the pig (host) cells, including cardiomyocytes and other cell types. However, none of these genes were differentially expressed, with a minimum of 2-fold difference between the Spheroid and I/R Groups at 1 week. Therefore, we hypothesized that the increased cell-cycle in Spheroid Group at 1 week was associated with the extracellular proteins that were not secreted by the pig cells, but by the transplanted ^{HLA-KO/CCND2}hiPSC-CM. Then, the expression of 39 extracellular genes, which did not express in the pig snRNAseq data, were examined in hiPSC-CM bulk-RNA expression dataset (Wang L, et al. Front Bioeng Biotechnol. 2023 11:1108340); here, there were only 7 genes having the minimum FPKM of 1.0: Inhibin Subunit Beta E [INHBE], Left-Right Determination Factor 2 [LEFTY2], Nodal Growth Differentiation Factor [NODAL], Bone Morphogenetic Protein 7 [BMP7], Follistatin [FST], Fibroblast Growth Factor 8 [FGF8], and Fibroblast Growth Factor 21 [FGF21] (FIG. 27). Thus, we examined the paracrine factor profile to identify potential factors that induced porcine CM proliferation after ^{HLA-KO/CCND2-OE}hiPSC-CM spheroid transplantation.

^{HLA-KO/CCND2-OE}hiPSC-CM Spheroids Produced More Follistatin than ^{WT}hiPSC-CM Spheroids

Human angiogenesis arrays showed that 7 proteins were significantly reduced by the ^{HLA-KO/CCND2-OE}hiPSC-CM spheroids as compared with the ^{WT}hiPSC-CM spheroids. On the contrary, 14 protein-products were significantly increased by the ^{HLA-KO/CCND2-OE}hiPSC-CM spheroids than the ^{WT}hiPSC-CM spheroids. Among the 14 proteins, follistatin was found to be increased by 82.2% in ^{HLA-KO/CCND2-OE}hiPSC-CM spheroids as compared to ^{WT}hiPSC-CM spheroids (p<0.01), suggesting that follistatin may be a key factor contributed to increased porcine CM cell-cycle activity. Thus, we assessed the effect of follistatin on CM proliferation.

Follistatin Promoted hiPSC-CM Proliferation Through Modulating YAP Signaling Pathway

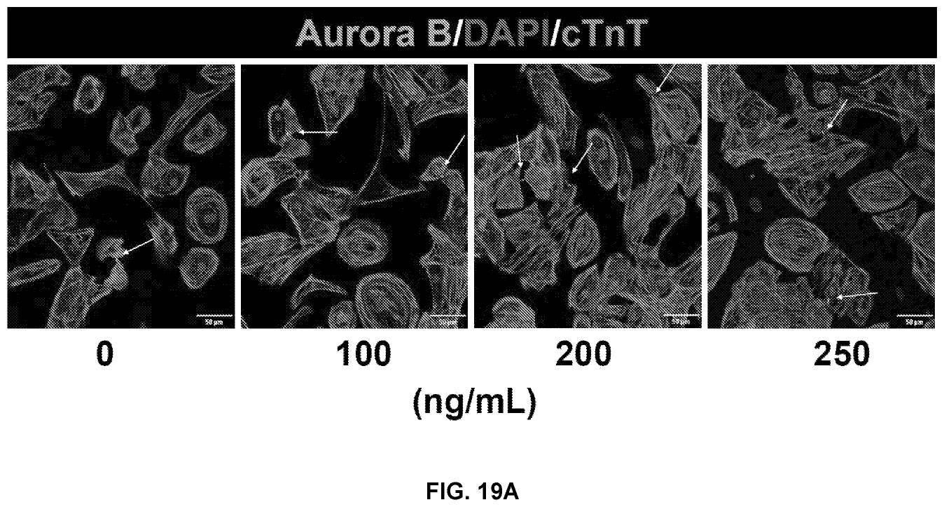

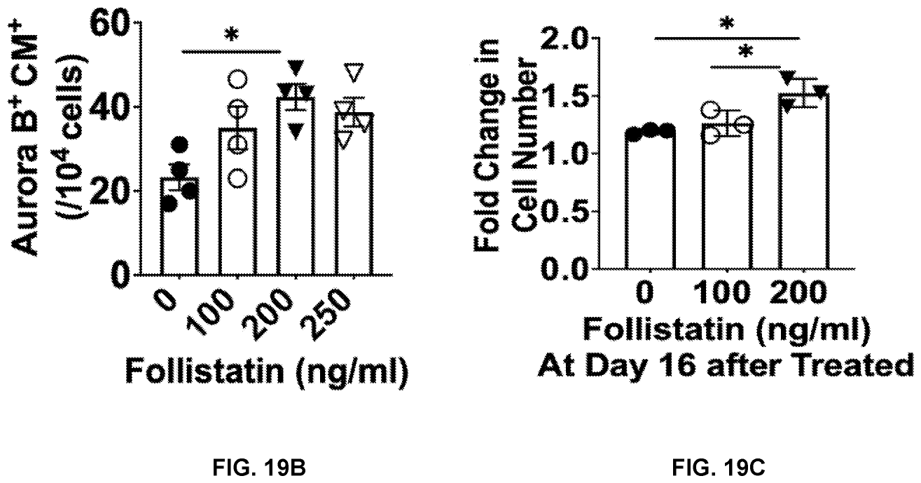

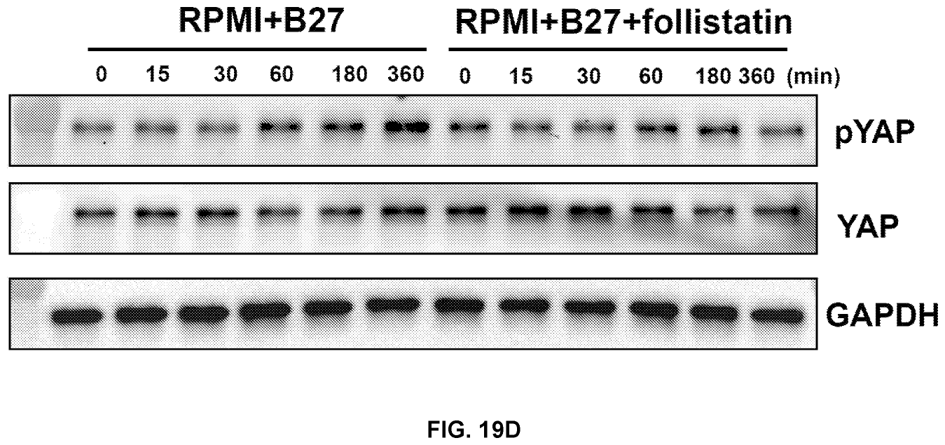

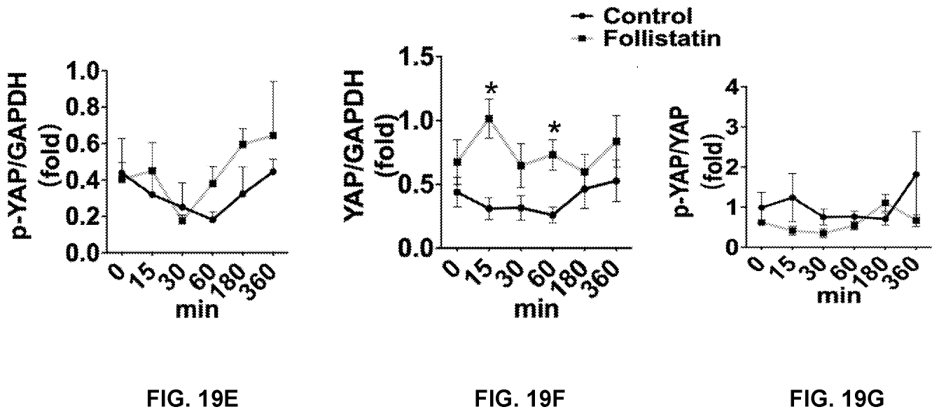

First, the dose dependent effect of follistatin on hiPSC-CM cytokinesis was assessed. Although starting from 100 ng/mL follistatin already increased the symmetric ABK⁺ hiPSC-CM number, only 200 ng/mL follistatin significantly induced cytokinesis in ^{WT}hiPSC-CMs (FIGS. 19A & 19B). Continuously culturing ^{WT}hiPSC-CMs with 200 ng/mL Follistatin for 16 days significantly increased CM number (FIG. 19C). To understand how follistatin could affect YAP signaling pathway, pYAP and YAP expressions were assessed in a time-course dependent manner (FIG. 19D). Western Blot showed that follistatin significantly increased total YAP protein expression level at min and 60 min after follistatin treatment (FIG. 19F). These data suggest that follistain can increase YAP protein expression through which to promote CM proliferation. Thus, follistatin secreted from the transplanted ^{HLA-KO/CCND2-OE}hiPSC-CM spheroids may up-regulate total YAP protein expression in porcine CMs to induce CM cytokinesis.

### DISCUSSION

Low proliferative capacity of hiPSC-CMs and immunorejection limit therapeutic efficacy of hiPSC-CMs for the treatment of myocardial injury [68]. To overcome these, we generated a line of ^{HLA-KO/CCND2-OE}hiPSC. In the current study, we assessed the therapeutic effects of ^{HLA-KO/CCND2-OE}hiPSC-CM) spheroids for the treatment of myocardial injury in a pig heart model of I/R. We found that transplantation of ^{HLA-KO/CCND2-OE}hiPSC-CM spheroids significantly improved heart function and reduced scar size. The structural and functional improvement was associated with significantly reduced pYAP expression and increased pig CM proliferation which might be stimulated by follistatin released from ^{HLA-KO/CCND2-OE}hiPSC-CMs.

Cardiac MRI data indicated that both LVESV and LVEDV were significantly smaller in the Spheroid Group as compared to the Control Group, indicating dilatation of LV after I/R was reduced by CM spheroid transplantation. The global systolic function as indicated by LVEF and scar size as indicated by late gadolinium enhancement imaging were better in the Spheroid Group than the Control Group, suggesting that spheroid transplantation may induced porcine CM proliferation through which reduced fibrotic tissue formation and enhanced systolic function recovery.

Fluorescence immunostaining confirmed that not only mitosis of porcine CM significantly increased by 3.8 folds, but also cytokinesis increased by 6.3 folds in porcine hearts of the Spheroid Group as compared with the I/R Group at 1 week after I/R and treatment. In addition, the Sham group had the highest expression levels of pLATS1 and pYAP, while spheroid transplantation had the lowest expression levels of the of pLATS1 and pYAP. YAP immunostaining confirmed that more non-phosphorylated YAP proteins were found in porcine CM nuclei, where YAP can activate downstream genes responsible for cell proliferation (von Gise A, et al. Proc Natl Acad Sci USA. 2012 109(7):2394-9). Thus, ^{HLA-KO/CCND2-OE}hiPSC-CM spheroid transplantation may reduce pYAP and promote YAP translocation to porcine CM through which promotes porcine CM proliferation.

To understand the mechanism of how ^{HLA-KO/CCND2-OE}hiPSC-CM spheroid transplantation regulate porcine myocardial YAP, we first analyzed snRNAsqeq data and found that a cycling CM population, which had five proliferation marker cohocalized, was in the Spheroid Group which was >5 folds of that in the I/R Group at 1 week. Analyzing pro-proliferation signaling pathways, we found that only HIPPO/YAP, TGFβ, and MAPK pathways were significantly upregulated in the Spheroid Group. Totally 56 extracellular ligand genes were identified through respective corresponding receptors can activate HIPPO/YAP, TGFβ, and MAPK pathways.

Further analyzing the 56 extracellular ligand genes, 17 genes were not differentially expressed in porcine cardiac cells and 39 genes were not identified in the pig snRNAseq data. When combined with hiPSC-CM bulk-RNA expression dataset (Wang L, et al. Front Bioeng Biotechnol. 2023 11:1108340), BMP7 and follistatin were identified to be involved in HIPPO/YAP signaling pathway. Interestingly, 4×10^{6 HLA-KO/CCND2-OE} hiPSC-CMs were able to secrete up to 30.3 ng/mL follistatin in vitro. When 25×10^{6 HLA-KO/CCND2-OE}hiPSC-CMs were transplanted, the peak follistatin may reach 189.4 ng/mL in cell transplanted area. This concentration is close to in vitro experiments showing that 200 ng/mL most efficiently induced hiPSC-CM cytokinesis.

Follistatin is a member of the tissue growth factor 13 family and is a secreted glycoprotein and has been to promote skeletal muscle (Yaden B C, et al. J Pharmacol Exp Ther. 2014 349(2):355-71) and liver (Kogure K, et al. Gastroenterology. 1995 108(4):1136-42) regeneration. In vitro studies showed that follistatin had a dose dependent effect on CM proliferation. 200 ng/mL follistatin significantly increased numbers of ABK⁺ hiPSC-CMs under cytokinesis. When hiPSC-CMs were continuously co-cultured with 200 ng/mL follistatin for 16 days, the numbers of hiPSC-CMs increased by 50%. More importantly, follistatin significantly increased total YAP protein expression in hiPSC-CMs as soon as 15 min after co-culture, suggesting that follistatin is able to induce YAP protein synthesis and does not induce pYAP in CMs. This is different from porcine tissue western blot results which showed that pYAP expression was reduced in ^{HLA-KO/CCND2-OE}hiPSC-CM spheroid transplanted porcine myocardium. This difference may be due to the different samples were used. We used whole porcine myocardial tissues for western blot, which contained cardiac cells, including fibroblasts, smooth muscle cells, and endothelial cells. These cells may have different responses to I/R or follistatin, which may compromise the real effect of follistatin on total YAP protein expression in porcine CMs.

The engraftment of ^{HLA-KO/CCND2-OE}hiPSC-CMs were only found in porcine hearts at 1 week, not 4 weeks after I/R and treatment, suggesting that even human hiPSC-CMs with HLA-I and -II knock-down were still not able to escape porcine immune-reaction and the combination of immune-suppressive drugs used in pig in this study is yet satisfactory. However, it is still possible that this cell line may be still hypo-immunogenic in patients while porcine immune system is still able to recognize HLA-I and -II knock-down xenotransplated human cells.

In conclusion, transplantation of the ^{HLA-KO/CCND2-OE}hiPSC-CM spheroids significantly improved heart function, reduced scar size, induced porcine CM proliferation, and limited pYAP expression in the porcine hearts after I/R. The increased porcine CM proliferation may be stimulated by follistatin released from ^{HLA-KO/CCND2-OE} hiPSC-CMs. This cell line has important clinical implications for the treatment of MI: a). CMs differentiated from this cell line are hypo-immunogenic which make them suitable for allogenic transplantation; b). follistatin secreted by ^{HLA-KO/CCND2-OE}hiPSC-CM spheroids promotes CM regeneration. These properties make this cell line an attractive therapeutic for the treatment of myocardial I/R.

### Example 3: Implanted Cardiac Spheroids Survive after One Week and Exhibit Electrical Coupling with the Swine Host Myocardium

Adult mammalian heart tissue lacks regenerative capacity to replenish cardiomyocytes lost in myocardial infarction (MI); instead, the damaged heart undergoes extensive remodeling that impairs normal cardiac function and may eventually lead to heart failure (Hashimoto H, et al. Nature reviews. Cardiology. 2018 15(10):585). A recent strategy to remuscularize infarcts is to implant engineered tissue fabricated from human induced pluripotent stem cell-derived cardiomyocytes (hiPSC-CMs) (Zhao M T, et al. Frontiers in Cell and Developmental Biology. 2020 8:1142). Large animal models have shown structural integration with improved cardiac function (Ye L, et al. Cell stem cell. 2014 15(6):750-761). Electrical coupling with the host is a critical factor for functional integration of the implant as well as for arrythmia risk, yet is understudied. Here, we use optical mapping to document electrical coupling of engineered tissue grafts with the host heart in a large animal model.

We obtained 3 domestic farm pigs of either sex weighing 15-20 kg, and gave the immunosuppressant cyclosporine for 5 days (45 mg/kg/day). We then anesthetized the animals, opened the chest, and created an anterior MI by occluding the left anterior descending coronary artery for one hour. After reperfusion, we implanted engineered tissue into the MI border zone. The tissue consisted of hiPSC-CMs spheroids (200-250 μm size) (Kahn-Krell A, et al. Frontiers in Bioengineering and Biotechnology. 2021 9:494) transfected with GCaMP6, a fluorescent calcium indicator. The spheroids (containing a total of 50 million cardiomyocytes) were implanted in 4 columns perpendicular to the epicardial surface by injecting a spheroid suspension while slowly withdrawing the needle.

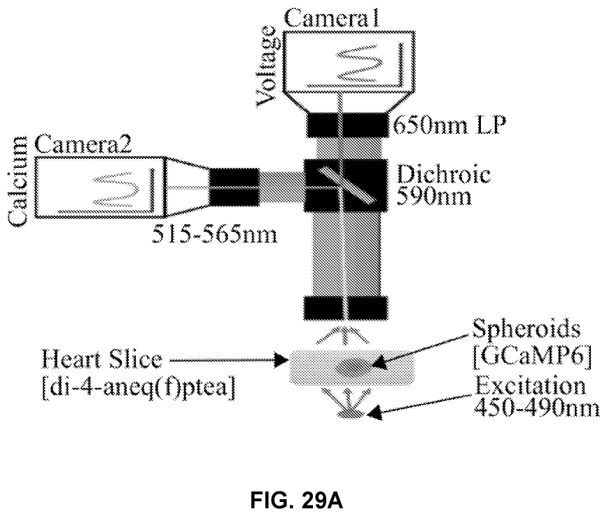

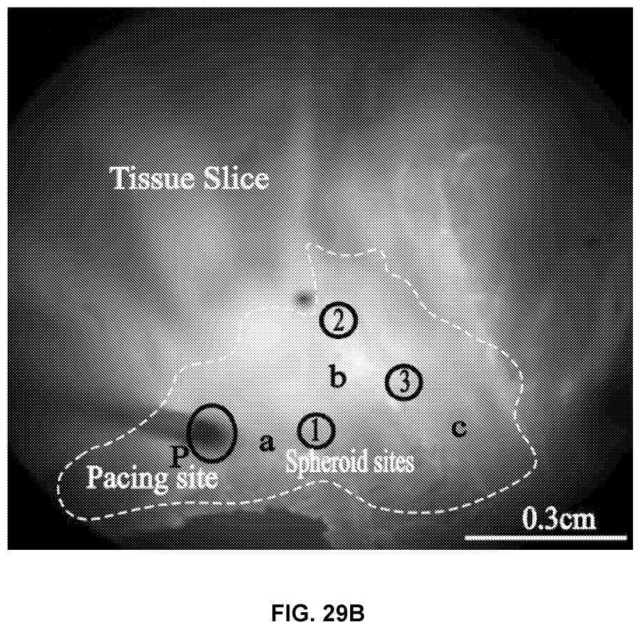

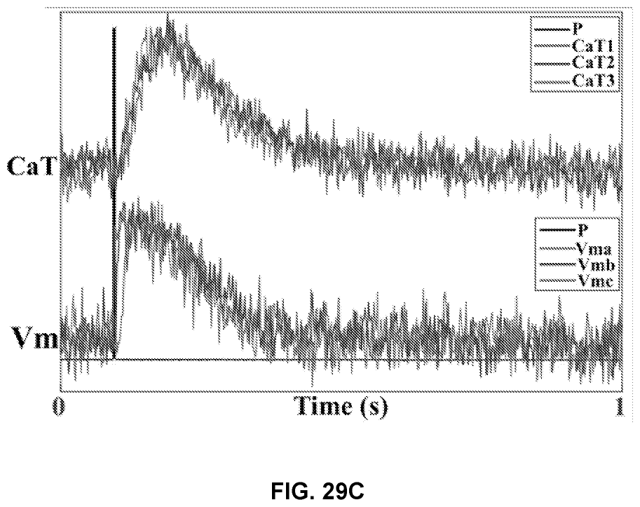

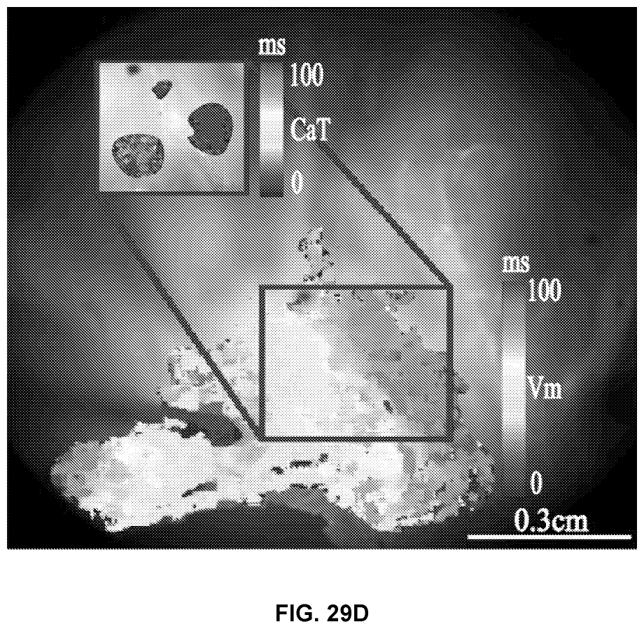

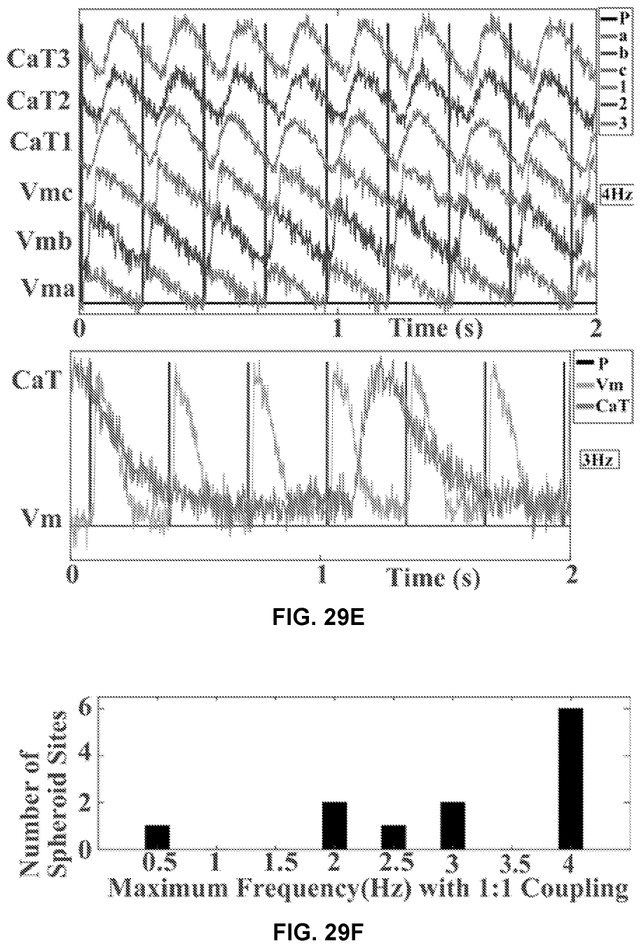

We imaged electrical activity in the host and the intramural grafts 1 week after implant. Electrical activity within the heart wall is normally imaged using needle-based electrodes or optrodes. However, this approach lacks sufficient spatial resolution, so we instead performed optical mapping5 of tissue slices taken parallel to the epicardium and perpendicular to the spheroid columns. The pigs were sacrificed, and the hearts harvested under ice cold cardioplegia. An approximately 1.5 cm×1.5 cm block of left ventricular tissue was excised from the implant zone of each heart. Heart tissue slices of 600 μm thickness were cut parallel to the epicardium using a high precision vibratome (700smz-2, Campden Instruments Ltd. UK) and maintained in oxygenated Tyrodes solution before mapping. The slices were stained with a voltage sensitive dye, di-4-aneq(f)ptea (Potentiometric Probes), and excited using blue LEDs (Luxeon C, Lumileds) to optically map transmembrane potential (Vm) from the tissue slices and calcium transients (CaT) from the implanted GCaMP6-spheroids simultaneously (FIG. 29A, 29B).

To detect the presence of surviving engineered tissue in each slice, we applied field stimulation to excite the entire preparation. Because only spheroids expressed GCaMP6, the presence of CaT signal indicated spheroid survival (FIG. 29C). We identified 21 surviving spheroid sites from 16 slices (1, 12, and 3 slices obtained respectively from 3 pigs). Field stimulation directly activated slice and engrafted spheroids simultaneously (FIG. 29C). To detect electrical coupling between host and spheroids, we focally paced the slices (at twice diastolic threshold) from a point distant from the spheroid sites (e.g., FIG. 29B). In this situation, spheroid activation (indicated by a CaT signal) should occur when the site is reached by a wave propagating through the slice and not at the time of stimulation. Such coupling was clearly evident at 12 spheroid sites in 10 slices from 3 pigs. FIG. 29D is an example. We did not observe coupling at the remaining spheroid sites, even though slices and spheroids were both active.

To assess coupling efficacy, we gradually increased the pacing rate from 0.5 Hz to 4 Hz with step size of 0.5 Hz. Six spheroid sites from 5 slices maintained 1:1 coupling even at 4 Hz (FIG. 29E). At other sites, 1:1 coupling was lost at lower rates (FIG. 29F).

Here, we find that cardiac tissue grafts engineered from human induced pluripotent stem cells survive 1 week of implant in immunosuppressed pigs and remain electrically active. Furthermore, they can electrically couple with the host tissue and can be driven at rates up to 4 Hz. Some of the spheroid sites we identified were not coupled with the host. This could be because they were surrounded by infarct scar in the plane of the slice. This issue could be addressed by implanting engineered tissue in non-infarcted myocardium. It is also possible that some grafts require longer implant time to integrate. In this study, we did not investigate propagation from the graft to the host. This is an important issue because it could enable graft involvement in detrimental arrythmias. To our knowledge, this is the first direct demonstration of electrical coupling between a host and an engineered graft in a large animal. It shows that grafts can be driven by the host, which is a critical prerequisite for the graft to re-muscularize the heart and synchronously contribute to cardiac contraction.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A method for producing a cardiac spheroid, comprising
(a) culturing pluripotent stem cells under conditions suitable to induce differentiation into cardiomyocyte (CM) spheroids;
(b) culturing pluripotent stem cells under conditions suitable to induce differentiation into endothelial cells (ECs);
(c) culturing pluripotent stem cells under conditions suitable to induce differentiation into smooth muscle cells (SMCs);
(d) culturing pluripotent stem cells under conditions suitable to induce differentiation into cardiac fibroblasts (CFs);
(e) dissociating the CM spheroids, ECs, SMCs, and CFs to produce CM, EC, SMC, and CF cell suspensions;
(f) mixing the CM, ECs, SMCs, and CFs cell suspensions at a CM:EC:SMC:CF 4:2:1:1 ratio to produce a cardiac cell mixture; and
(g) culturing the cardiac cell mixture under conditions suitable to form cardiac spheroids.

2. The method of claim 1, wherein the pluripotent stem cells are induced pluripotent cells (iPSCs).

3. The method of claim 2, wherein the iPSCs are engineered to overexpress cell-cycle regulatory gene cyclin D2 (CCND2).

4. The method of claim 3, wherein the iPSCs are engineered to contain a heterologous CCND2 gene operably linked to a myosin heavy chain (MHC) promoter.

5. The method of claim 2, wherein the iPSCs are engineered to delete one or more human leukocyte antigen (HLA) genes.

6. The method of claim 5, wherein the iPSCs are engineered to delete HLA-I and HLA-II genes.

7. A cardiac spheroid produced by the method of claim 1.

8. A composition comprising the cardiac spheroid of claim 7 in a pharmaceutically acceptable excipient.

9. A method for treating a subject with a myocardial infarction, comprising administering to the heart of the subject the composition of claim 8.
